(19) United States
(12) Patent Application Publication
(10) Pub. No.: US 2004/0018574 A1
(43) Pub. Date: Jan. 29, 2004

(54) Molecular differences between species of the M. tuberculosis complex

(76) Inventors: Marcel Behr, Montreal (CA); Peter Small, Seattle, WA (US); Michael A. Wilson, Bethesda, MD (US); Gary Schoolnik, Stanford, CA (US); Claus Aagaard, Kobenhavn S (DK); Ida Rosenkrands, Vaerlose (DK); Karin Weldingh, Vaerlose (DK); Peter Andersen, Bronshoj (DK)

Correspondence Address: BOZICEVIC, FIELD & FRANCIS LLP, 200 MIDDLEFIELD RD, SUITE 200, MENLO PARK, CA, 94025, US

(21) Appl. No.: 10/388,902

(22) Filed: Mar. 14, 2003

Related U.S. Application Data
(63) Continuation-in-part of application No. 09/894,844, filed on Jun. 27, 2001.
Application No. 09/894,844 is a continuation of application No. 09/318,191, filed on May 25, 1999, now Pat. No. 6,291,190.
(60) Provisional application No. 60/097,936, filed on Aug. 25, 1998.

(51) Int. Cl. G01N 33/554; G01N 33/569; C07K 14/35; A61K 39/02

(52) U.S. Cl. 435/7.32; 530/350; 424/190.1

(57) ABSTRACT

Specific genetic deletion are identified in mycobacteria isolates, including variations in the M. tuberculosis genome sequence between isolates, and numerous deletion present in BCG as compared to M. tbThe genetic markers can be used for diagnosis of M. tb. infection. One or more antigens provided from the genetic markers can be used in diagnostic assays, e.g. a serological assay.

This invention was made with Government support under contract AI01137, AI35969 awarded by the National Institutes of Health. The Government has certain rights in this invention.

Tuberculosis remains a major health problem, with two million deaths and eight million new cases annually. At the same time, one third of the total world population—two billion people—are infected with the etiological agent, Mycobacterium tuberculosis, and have a 10% lifetime risk of progressing from infection to clinical disease. Although tuberculosis can be treated, an estimated 2.9 million people died from the disease last year.

There are significant problems with a reliance on drug treatment to control active M. tuberculosis infections. Most of the regions having high infection rates are less developed countries, which suffer from a lack of easily accessible health services, diagnostic facilities and suitable antibiotics against M. tuberculosis. Even where these are available, patient compliance is often poor because of the lengthy regimen required for complete treatment, and multidrug-resistant strains are increasingly common.

Prevention of infection would circumvent the problems of treatment, and so vaccination against tuberculosis is widely performed in endemic regions. Around 100 million people a year are vaccinated with live bacillus Calmette-Guerin (BCG) vaccine. BCG has the great advantage of being inexpensive and easily administered under less than optimal circumstances, with few adverse reactions. Unfortunately, the vaccine is widely variable in its efficacy, providing anywhere from 0 to 80% protection against infection with M. tuberculosis.

BCG has an interesting history. It is an attenuated strain of M. bovis, a very close relative of M. tuberculosis. The M. bovis strain that became BCG was isolated from a cow in the late 1800's by a bacteriologist named Nocard, hence it was called Nocard's bacillus. The attenuation of Nocard's bacillus took place from 1908 to 1921, over the course of 230 in vitro passages. Thereafter, it was widely grown throughout the world, resulting in additional hundreds and sometime thousands of in vitro passages. Throughout its many years in the laboratory, there has been selection for cross-reaction with the tuberculin skin test, and for decreased side effects. The net results have been a substantially weakened pathogen, which may be ineffective in raising an adequate immune response.

Relevant Literature

Mahairas et al. (1996) J Bacteriol 178(5):1274-1282 provides a molecular analysis of genetic differences between Mycobacterium bovis BCG and virulent M. bovis. Subtractive genomic hybridization was used to identify genetic differences between virulent M. bovis and M. tuberculosis and avirulent BCG. U.S. Pat. No. 5,700,683 is directed to these genetic differences.

Cole et al. (1998) Nature 393:537-544 have described the complete genome of M. tuberculosis. To obtain the contiguous genome sequence, a combined approach was used that involved the systematic sequence analysis of selected large-insert clones as well as random small-insert clones from a whole-genome shotgun library. This culminated in a composite sequence of 4,411,529 base pairs, with a G+C content of 65.6%. 3,924 open reading frames were identified in the genome, accounting for ˜91% of the potential coding capacity. Difficulties with conventional serological assays for tuberculosis are reviewed by Pottumarthy et al. (2000) J Clin Microbiol 38(6):2227-31. Trajman et al. (1997) Int. J. Tuberc. Lung Dis. 1, 498-501 discloses the co-incidence of HIV and TB, and reviews issues of diagnostic assays in these patients. Cole et al. (1996) Tuber Lung Dis 77(4):363-8; Lyashchenko et al (1998) Infection and Immunity 66 (8): 3936-3940 describes differences in the response to antigens by different patient populations.

Mycobacterium tuberculosis (M.tb.) genomic sequence is available at several internet sites. Included is Rv0934, Genbank accession NC_{—}000962, nt. 1042113-1043237.

### SUMMARY OF THE INVENTION

Genetic markers are provided that distinguish between strains of the Mycobacterium tuberculosis complex, particularly between avirulent and virulent strains. Strains of interest include M. bovis, M. bovis BCG strains, M. tuberculosis (M. tb.) isolates, and bacteriophages that infect mycobacteria. The genetic markers are used for assays, e.g. immunoassays, that distinguish between strains, such as to differentiate between BCG immunization and M. tb. infection.

In one embodiment of the invention, a plurality of antigens from the provided genetic markers is used in the diagnosis of M. tuberculosis infection. The antigens may be used in a serological, or a cell-mediated diagnostic assay, usually serological. Antigens may be combined in a single test sample, or may be separately assayed with the data compiled prior to diagnosis. Alternatively the markers of interests can be produced as recombinant fusion proteins, comprising at least one epitope from one marker and at least one epitope from a second marker. The resulting fusion molecule can be used in the diagnostic assays. Antigens of interest for such diagnosis include combinations of two or more of Rv1516c (SEQ ID NO: 53), Rv1575 (SEQ ID NO: 32), Rv1586c (SEQ ID NO: 21), Rv2660c (SEQ ID NO: 100), Rv3118 (SEQ ID NO: 39), Rv1976c (SEQ ID NO: 122), Rv1966 (SEQ ID NO: 112), Rv3618 (SEQ ID NO: 71) and Rv3429 (SEQ ID NO: 76), which are optionally combined with r38 kDa protein (Rv0934; Bothamley and Rudd (1994)). Preferred antigens are not cross-reactive with environmental mycobacteria antigens, e.g. with M. avium antigens.

### DETAILED DESCRIPTION OF THE EMBODIMENTS

Specific genetic deletions are identified that serve as markers to distinguish between avirulent and virulent mycobacteria strains, including M. bovis, M. bovis BCG strains, M. tuberculosis (M. tb.) isolates, and bacteriophages that infect mycobacteria. These deletions are used as genetic markers to distinguish between the different mycobacteria. The deletions may be introduced into M. tb. or M. bovis by recombinant methods in order to render a pathogenic strain avirulent. Alternatively, the deleted genes are identified in the M. tb. genome sequence, and are then reintroduced by recombinant methods into BCG or other vaccine strains, in order to improve the efficacy of vaccination.

The deletions of the invention are identified by comparative DNA hybridizations from genomic sequence of mycobacterium to a DNA microarray comprising representative sequences of the M. tb. coding sequences. The deletions are then mapped to the known M. tb. genome sequence in order to specifically identify the deleted gene(s), and to characterize nucleotide sequence of the deleted region.

In one embodiment of the invention, a plurality of antigens encoded by the provided genetic markers is used in the diagnosis of M. tuberculosis infection. One or more antigenic polypeptides or fragments thereof encoded by the provided deletion markers, herein collectively referred to as “antigens” or “deletion antigens” can be used in diagnostic assays. The antigens may be combined in a single test format, e.g. well, tube, etc., for use in the analysis of binding or immunoreactivity with a patient sample. Alternatively the antigens are assayed in separate test wells, tubes, etc., and the results collated or compiled prior to diagnosis. Antigens can be separately synthesized, e.g. by recombinant or chemical methods, or epitopes from different markers can be combined and produced in a fusion protein.

Serologic assays may be used. Such assays determine whether a patient sample, e.g. blood, lymph, saliva, etc., contains antibodies that specifically bind to deletion antigens, where the presence of such antibodies is indicative of prior, or current, infection with a virulent strain of the M. tuberculosis complex. Antigens of interest for such assays include combinations of one or more of Rv1516c (SEQ ID NO: 53), Rv1575 (SEQ ID NO: 32), Rv1586c (SEQ ID NO: 21), Rv2660c (SEQ ID NO: 100), Rv3118 (SEQ ID NO: 39), Rv1976c (SEQ ID NO: 122), Rv1966 (SEQ ID NO: 112), Rv3618 (SEQ ID NO: 71) and Rv3429 (SEQ ID NO: 76), which are optionally combined with r38 kDa protein. Assays may use 2 of the deletion antigens, 3 of the deletion antigens, 4 of the deletion antigens, 5, 6, 7, 8 or 9 of the deletion antigens. A preferred diagnostic reagent will provide high sensitivity.

Specific combinations of interest include, without limitation, Rv1586c and Rv3118; Rv1586c and Rv3618; Rv1586c, Rv3118 and Rv3618; Rv1516c, Rv1586c, Rv3118 and Rv3618; Rv1586c, Rv1976c, Rv3118 and Rv3618; Rv1586c, Rv2660c, Rv3118 and Rv3618; and Rv1586c, Rv2660c, Rv3118, Rv3618 Rv1966, Rv3429 and Rv3618. In certain populations, the antigens Rv1966, Rv3429 and Rv3618 are recognized by more than 50% of the HIV negative TB patients and Rv3429 is recognized by 85% of the HIV positive donors.

Definitions

By the term “virulent Mycobacterium” is understood a bacterium capable of causing the tuberculosis disease in a mammal including a human being. Examples of virulent Mycobacteria are M. tuberculosis, M. africanum, and M. Bovis.

By a “TB patient” is understood an individual with culture or microscopically proven infection with virulent Mycobacterium, and/or an individual clinically diagnosed with TB and who is responsive to anti-TB chemotherapy. Culture, microscopy and clinical diagnosis of TB is well known by a person of skill in the art.

By the term “PPD positive individual” is understood an individual with a positive Mantoux test or an individual where PPD (purified protein derivative) induces an increase in in vitro recall response determined by release of γIFN (gamma interferon) of at least 1,000 pg/ml from peripheral blood mononuclear cells (PBMC) or whole blood, the induction being performed by the addition of 2.5 to 5 μg of PPD/ml to a suspension comprising about 1.0 to 2.5×10⁵ PBMC, the release of γIFN being assessable by determination of γIFN in supernatant harvested 5 days after the addition of PPD to the suspension compared to the release of γIFN without the addition of PPD.

By the term “delayed type hypersensitivity reaction” is understood a T cell mediated inflammatory response elicited after the injection of a polypeptide into or application to the skin, the inflammatory response appearing 72-96 hours after the polypeptide injection or application.

Identification of M. tuberculosis Complex Deletion Markers

The present invention provides nucleic acid sequences that are markers for specific mycobacteria, including M. tb., M. bovis, BCG and bacteriophage. The deletions are listed in Table 1. The absence or presence of these marker sequences is characteristic of the indicated isolate, or strain. As such, they provide a unique characteristic for the identification of the indicated mycobacteria. The deletions are identified by their M. tb. open reading frame (“Rv” nomenclature), which corresponds to a known genetic sequence, and may be accessed as previously cited. The junctions of the deletions are provided by the designation of position in the publicly available M. tb. sequence.

TABLE 1

| SEQ ID | rd | rv_num | orf_id | breakpoint |
|---|---|---|---|---|
| SEQ ID NO: 1 | RD01 | Rv3871 | MTV027.06 | “H37Rv, segment 160: 7534, 16989” |
| SEQ ID NO: 2 | RD01 | Rv3872 | MTV027.07 | “H37Rv, segment 160: 7534, 16989” |
| SEQ ID NO: 3 | RD01 | Rv3873 | MTV027.08 | “H37Rv, segment 160: 7534, 16989” |
| SEQ ID NO: 4 | RD01 | Rv3874 | MTV027.09 | “H37Rv, segment 160: 7534, 16989” |
| SEQ ID NO: 5 | RD01 | Rv3875 | MTV027.10 | “H37Rv, segment 160: 7534, 16989” |
| SEQ ID NO: 6 | RD01 | Rv3876 | MTV027.11 | “H37Rv, segment 160: 7534, 16989” |
| SEQ ID NO: 7 | RD01 | Rv3877 | MTV027.12 | “H37Rv, segment 160: 7534, 16989” |
| SEQ ID NO: 8 | RD01 | Rv3878 | MTV027.13 | “H37Rv, segment 160: 7534, 16989” |
| SEQ ID NO: 9 | RD01 | Rv3879c | MTV027.14c | “H37Rv, segment 160: 7534, 16989” |
| SEQ ID NO: 10 | RD02 | Rv1988 | MTCY39.31c | “H37Rv segment 88: 14211, segment 89: 8598” |
| SEQ ID NO: 11 | RD02 | Rv1987 | MTCY39.32c | “H37Rv segment 88: 14211, segment 89: 8598” |
| SEQ ID NO: 12 | RD02 | Rv1986 | MTCY39.33c | “H37Rv segment 88: 14211, segment 89: 8598” |
| SEQ ID NO: 13 | RD02 | Rv1985c | MTCY39.34 | “H37Rv segment 88: 14211, segment 89: 8598” |
| SEQ ID NO: 14 | RD02 | Rv1984c | MTCY39.35 | “H37Rv segment 88: 14211, segment 89: 8598” |
| SEQ ID NO: 15 | RD02 | Rv1983 | MTCY39.36c | ”H37Rv segment 88: 14211, segment 89: 8598” |
| SEQ ID NO: 16 | RD02 | Rv1982c | MTCY39.37 | “H37Rv segment 88: 14211, segment 89: 8598” |
| SEQ ID NO: 17 | RD02 | Rv1981c | MTCY39.38 | “H37Rv segment 88: 14211, segment 89: 8598” |
| SEQ ID NO: 18 | RD02 | Rv1980c | MTCY39.39 | “H37Rv segment 88: 14211, segment 89: 8598” |
| SEQ ID NO: 19 | RD02 | Rv1979c | MTCY39.40 | “H37Rv segment 88: 14211, segment 89: 8598” |
| SEQ ID NO: 20 | RD02 | Rv1978 | MTV051.16 | “H37Rv segment 88: 14211, segment 89: 8598” |
| SEQ ID NO: 21 | RD03 | Rv1586c | MTCY336.18 | “H37Rv, segment 70: 7677, 16923” |
| SEQ ID NO: 22 | RD03 | Rv1585c | MTCY336.19 | “H37Rv, segment 70: 7677, 16923” |
| SEQ ID NO: 23 | RD03 | Rv1584c | MTCY336.20 | “H37Rv, segment 70: 7677, 16923” |
| SEQ ID NO: 24 | RD03 | Rv1583c | MTCY336.21 | “H37Rv, segment 70: 7677, 16923” |
| SEQ ID NO: 25 | RD03 | Rv1582c | MTCY336.22 | “H37Rv, segment 70: 7677, 16923” |
| SEQ ID NO: 26 | RD03 | Rv1581c | MTCY336.23 | “H37Rv, segment 70: 7677, 16923” |
| SEQ ID NO: 27 | RD03 | Rv1580c | MTCY336.24 | “H37Rv, segment 70: 7677, 16923” |
| SEQ ID NO: 28 | RD03 | Rv1579c | MTCY336.25 | “H37Rv, segment 70: 7677, 16923” |
| SEQ ID NO: 29 | RD03 | Rv1578c | MTCY336.26 | “H37Rv, segment 70: 7677, 16923” |
| SEQ ID NO: 30 | RD03 | Rv1577c | MTCY336.27 | “H37Rv, segment 70: 7677, 16923” |
| SEQ ID NO: 31 | RD03 | Rv1576c | MTCY336.28 | “H37Rv, segment 70: 7677, 16923” |
| SEQ ID NO: 32 | RD03 | Rv1575 | MTCY336.29c | “H37Rv, segment 70: 7677, 16923” |
| SEQ ID NO: 33 | RD03 | Rv1574 | MTCY336.30c | “H37Rv, segment 70: 7677, 16923” |
| SEQ ID NO: 34 | RD03 | Rv1573 | MTCY336.31c | “H37Rv, segment 70: 7677, 16923” |
| SEQ ID NO: 35 | RD04 | Rv0221 | MTCY08D5.16 | “H37Rv, segment 12: 17432, 19335” |
| SEQ ID NO: 36 | RD04 | Rv0222 | MTCY08D5.17 | “H37Rv, segment 12: 17432, 19335” |
| SEQ ID NO: 37 | RD04 | Rv0223c | MTCY08D5.18 | “H37Rv, segment 12: 17432, 19335” |
| SEQ ID NO: 38 | RD05 | Rv3117 | MTCY164.27 | “H37Rv, segment 135: 27437, 30212” |
| SEQ ID NO: 39 | RD05 | Rv3118 | MTCY164.28 | “H37Rv, segment 135: 27437, 30212” |
| SEQ ID NO: 40 | RD05 | Rv3119 | MTCY164.29 | “H37Rv, segment 135: 27437, 30212” |
| SEQ ID NO: 41 | RD05 | Rv3120 | MTCY164.30 | “H37Rv, segment 135: 27437, 30212” |
| SEQ ID NO: 42 | RD05 | Rv3121 | MTCY164.31 | “H37Rv, segment 135: 27437, 30212” |
| SEQ ID NO: 43 | RD06 | Rv1506c | MTCY277.28c | “H37Rv, segment 65: 23614, 36347” |
| SEQ ID NO: 44 | RD06 | Rv1507c | MTCY277.29c | “H37Rv, segment 65: 23614, 36347” |
| SEQ ID NO: 45 | RD06 | Rv1508c | MTCY277.30c | “H37Rv, segment 65: 23614, 36347” |
| SEQ ID NO: 46 | RD06 | Rv1509 | MTCY277.31 | “H37Rv, segment 65: 23614, 36347” |
| SEQ ID NO: 47 | RD06 | Rv1510 | MTCY277.32 | “H37Rv, segment 65: 23614, 36347” |
| SEQ ID NO: 48 | RD06 | Rv1511 | MTCY277.33 | “H37Rv, segment 65: 23614, 36347” |
| SEQ ID NO: 49 | RD06 | Rv1512 | MTCY277.34 | “H37Rv, segment 65: 23614, 36347” |
| SEQ ID NO: 50 | RD06 | Rv1513 | MTCY277.35 | “H37Rv, segment 65: 23614, 36347” |
| SEQ ID NO: 51 | RD06 | Rv1514c | MTCY277.36c | “H37Rv, segment 65: 23614, 36347” |
| SEQ ID NO: 52 | RD06 | Rv1515c | MTCY277.37c | “H37Rv, segment 65: 23614, 36347” |
| SEQ ID NO: 53 | RD06 | Rv1516c | MTCY277.38c | “H37Rv, segment 65: 23614, 36347” |
| SEQ ID NO: 54 | RD07 | Rv2346c | MTCY98.15c | “H37Rv, segment 103: 17622, 26584” |
| SEQ ID NO: 55 | RD07 | Rv2347c | MTCY98.16c | “H37Rv, segment 103: 17622, 26584” |
| SEQ ID NO: 56 | RD07 | Rv2348c | MTCY98.17c | “H37Rv, segment 103: 17622, 26584” |
| SEQ ID NO: 57 | RD07 | Rv2349c | MTCY98.18c | “H37Rv, segment 103: 17622, 26584” |
| SEQ ID NO: 58 | RD07 | Rv2350c | MTCY98.19c | “H37Rv, segment 103: 17622, 26584” |
| SEQ ID NO: 59 | RD07 | Rv2351c | MTCY98.20c | “H37Rv, segment 103: 17622, 26584” |
| SEQ ID NO: 60 | RD07 | Rv2352c | MTCY98.21c | “H37Rv, segment 103: 17622, 26584” |
| SEQ ID NO: 61 | RD07 | Rv2353c | MTCY98.22c | “H37Rv, segment 103: 17622, 26584” |
| SEQ ID NO: 62 | RD08 | Rv0309 | MTCY63.14 | “H37Rv, segment 16: 17018, 20446” |
| SEQ ID NO: 63 | RD08 | Rv0310c | MTCY63.15c | “H37Rv, segment 16: 17018, 20446” |
| SEQ ID NO: 64 | RD08 | Rv0311 | MTCY63.16 | “H37Rv, segment 16: 17018, 20446” |
| SEQ ID NO: 65 | RD08 | Rv0312 | MTCY63.17 | “H37Rv, segment 16: 17018, 20446” |
| SEQ ID NO: 66 | RD09 | Rv3623 | MTCY15C10.29c | “H37Rv, segment 153: 21131, segment 154: 2832” |
| SEQ ID NO: 67 | RD09 | Rv3622c | MTCY15C10.30 | “H37Rv, segment 153: 21131, segment 154: 2832” |
| SEQ ID NO: 68 | RD09 | Rv3621c | MTCY15C10.31 | “H37Rv, segment 153: 21131, segment 154: 2832” |
| SEQ ID NO: 69 | RD09 | Rv3620c | MTCY15C10.32 | “H37Rv, segment 153: 21131, segment 154: 2832” |
| SEQ ID NO: 70 | RD09 | Rv3619c | MTCY15C10.33 | “H37Rv, segment 153: 21131, segment 154: 2832” |
| SEQ ID NO: 71 | RD09 | Rv3618 | MTCY15C10.34c | “H37Rv, segment 153: 21131, segment 154: 2832” |
| SEQ ID NO: 72 | RD09 | Rv3617 | MTCY15C10.35c | “H37Rv, segment 153: 21131, segment 154: 2832” |
| SEQ ID NO: 73 | RD10 | Rv1257c | MTCY50.25 | “H37Rv segment 55: 3689, 6696” |
| SEQ ID NO: 74 | RD10 | Rv1256c | MTCY50.26 | “H37Rv segment 55: 3689, 6696” |
| SEQ ID NO: 75 | RD10 | Rv1255c | MTCY50.27 | “H37Rv segment 55: 3689, 6696” |
| SEQ ID NO: 76 | RD11 | Rv3429 | MTCY77.01 | “H37Rv, segment 145: 30303 to segment 146: 1475” |
| SEQ ID NO: 77 | RD11 | Rv3428c | MTCY78.01 | “H37Rv, segment 145: 30303 to segment 146: 1475” |
| SEQ ID NO: 78 | RD11 | Rv3427c | MTCY78.02 | “H37Rv, segment 145: 30303 to segment 146: 1475” |
| SEQ ID NO: 79 | RD11 | Rv3426 | MTCY78.03c | “H37Rv, segment 145: 30303 to segment 146: 1475” |
| SEQ ID NO: 80 | RD11 | Rv3425 | MTCY78.04c | “H37Rv, segment 145: 30303 to segment 146: 1475” |
| SEQ ID NO: 81 | RD12 | Rv2072c | MTCY49.11c | “H37Rv segment 93: 9301, 11331” |
| SEQ ID NO: 82 | RD12 | Rv2073c | MTCY49.12c | “H37Rv segment 93: 9301, 11331” |
| SEQ ID NO: 83 | RD12 | Rv2074 | MTCY49.13 | “H37Rv segment 93: 9301, 11331” |
| SEQ ID NO: 84 | RD12 | Rv2075c | MTCY49.14c | “H37Rv segment 93: 9301, 11331” |
| SEQ ID NO: 85 | RD13bis | Rv2645 | MTCY441.15 | “H37Rv, segment 118: 12475, 23455” |
| SEQ ID NO: 86 | RD13bis | Rv2646 | MTCY441.16 | “H37Rv, segment 118: 12475, 23455” |
| SEQ ID NO: 87 | RD13bis | Rv2647 | MTCY441.17 | “H37Rv, segment 118: 12475, 23455” |
| SEQ ID NO: 88 | RD13bis | Rv2648 | MTCY441.17A | “H37Rv, segment 118: 12475, 23455” |
| SEQ ID NO: 89 | RD13bis | Rv2649 | MTCY441.18 | “H37Rv, segment 118: 12475, 23455” |
| SEQ ID NO: 90 | RD13bis | Rv2650c | MTCY441.19 | “H37Rv, segment 118: 12475, 23455” |
| SEQ ID NO: 91 | RD13bis | Rv2651c | MTCY441.20c | “H37Rv, segment 118: 12475, 23455” |
| SEQ ID NO: 92 | RD13bis | Rv2652c | MTCY441.21c | “H37Rv, segment 118: 12475, 23455” |
| SEQ ID NO: 93 | RD13bis | Rv2653c | MTCY441.22c | “H37Rv, segment 118: 12475, 23455” |
| SEQ ID NO: 94 | RD13bis | Rv2654c | MTCY441.23c | “H37Rv, segment 118: 12475, 23455” |
| SEQ ID NO: 95 | RD13bis | Rv2655c | MTCY441.24c | “H37Rv, segment 118: 12475, 23455” |
| SEQ ID NO: 96 | RD13bis | Rv2656c | MTCY441.25c | “H37Rv, segment 118: 12475, 23455” |
| SEQ ID NO: 97 | RD13bis | Rv2657c | MTCY441.26c | “H37Rv, segment 118: 12475, 23455” |
| SEQ ID NO: 98 | RD13bis | Rv2658c | MTCY441.27c | “H37Rv, segment 118: 12475, 23455” |
| SEQ ID NO: 99 | RD13bis | Rv2659c | MTCY441.28c | “H37Rv, segment 118: 12475, 23455” |
| SEQ ID NO: 100 | RD13bis | Rv2660c | MTCY441.29c | “H37Rv, segment 118: 12475, 23455” |
| SEQ ID NO: 101 | RD14 | Rv1766 | MTCY28.32 | “H37Rv segment 79: 30573, 39642” |
| SEQ ID NO: 102 | RD14 | Rv1767 | MTCY28.33 | “H37Rv segment 79: 30573, 39642” |
| SEQ ID NO: 103 | RD14 | Rv1768 | MTCY28.34 | “H37Rv segment 79: 30573, 39642” |
| SEQ ID NO: 104 | RD14 | Rv1769 | MTCY28.35 | “H37Rv segment 79: 30573, 39642” |
| SEQ ID NO: 105 | RD14 | Rv1770 | MTCY28.36 | “H37Rv segment 79: 30573, 39642” |
| SEQ ID NO: 106 | RD14 | Rv1771 | MTCY28.37 | “H37Rv segment 79: 30573, 39642” |
| SEQ ID NO: 107 | RD14 | Rv1772 | MTCY28.38 | “H37Rv segment 79: 30573, 39642” |
| SEQ ID NO: 108 | RD14 | Rv1773c | MTCY28.39 | “H37Rv segment 79: 30573, 39642” |
| SEQ ID NO: 109 | RD15 | Rv1963c | MTV051.01c | “H37Rv segment 88: 1153, 13873” |
| SEQ ID NO: 110 | RD15 | Rv1964 | MTV051.02 | “H37Rv segment 88: 1153, 13873” |
| SEQ ID NO: 111 | RD15 | Rv1965 | MTV051.03 | “H37Rv segment 88: 1153, 13873” |
| SEQ ID NO: 112 | RD15 | Rv1966 | MTV051.04 | “H37Rv segment 88: 1153, 13873” |
| SEQ ID NO: 113 | RD15 | Rv1967 | MTV051.05 | “H37Rv segment 88: 1153, 13873” |
| SEQ ID NO: 114 | RD15 | Rv1968 | MTV051.06 | “H37Rv segment 88: 1153, 13873” |
| SEQ ID NO: 115 | RD15 | Rv1969 | MTV051.07 | “H37Rv segment 88: 1153, 13873” |
| SEQ ID NO: 116 | RD15 | Rv1970 | MTV051.08 | “H37Rv segment 88: 1153, 13873” |
| SEQ ID NO: 117 | RD15 | Rv1971 | MTV051.09 | “H37Rv segment 88: 1153, 13873” |
| SEQ ID NO: 118 | RD15 | Rv1972 | MTV051.10 | “H37Rv segment 88: 1153, 13873” |
| SEQ ID NO: 119 | RD15 | Rv1973 | MTV051.11 | “H37Rv segment 88: 1153, 13873” |
| SEQ ID NO: 120 | RD15 | Rv1974 | MTV051.12 | “H37Rv segment 88: 1153, 13873” |
| SEQ ID NO: 121 | RD15 | Rv1975 | MTV051.13 | “H37Rv segment 88: 1153, 13873” |
| SEQ ID NO: 122 | RD15 | Rv1976c | MTV051.14 | “H37Rv segment 88: 1153, 13873” |
| SEQ ID NO: 123 | RD15 | Rv1977 | MTV051.15 | “H37Rv segment 88: 1153, 13873” |
| SEQ ID NO: 124 | RD16 | Rv3405c | MTCY78.23 | “H37Rv, segment 145: 5012, 12621” |
| SEQ ID NO: 125 | RD16 | Rv3404c | MTCY78.24 | “H37Rv, segment 145: 5012, 12621” |
| SEQ ID NO: 126 | RD16 | Rv3403c | MTCY78.25 | “H37Rv, segment 145: 5012, 12621” |
| SEQ ID NO: 127 | RD16 | Rv3402c | MTCY78.26 | “H37Rv, segment 145: 5012, 12621” |
| SEQ ID NO: 128 | RD16 | Rv3401 | MTCY78.27c | “H37Rv, segment 145: 5012, 12621” |
| SEQ ID NO: 129 | RD16 | Rv3400 | MTCY78.28c | “H37Rv, segment 145: 5012, 12621” |

The “Rv” column indicates public M. tb sequence, open reading frame. The BCG strains were obtained as follows:

TABLE 2
Strains employed in study of BCG phylogeny

| Name of strain | Synonym | Source | Descriptors |
|---|---|---|---|
| BCG-Russia | Moscow | ATCC | # 35740 |
| BCG-Moreau | Brazil | ATCC | # 35736 |
| BCG-Moreau | Brazil | IAF | dated 1958 |
| BCG-Moreau | Brazil | IAF | dated 1961 |
| BCG-Japan | Tokyo | ATCC | # 35737 |
| BCG-Japan | Tokyo | IAF | dated 1961 |
| BCG-Japan | Tokyo | JATA | vaccine strain |
| BCG-Japan | Tokyo | JATA | bladder cancer strain |
| BCG-Japan | Tokyo | JATA | clinical isolate-adenitis |
| BCG-Sweden | Gothenburg | ATCC | # 35732 |
| BCG-Sweden | Gothenburg | IAF | dated 1958 |
| BCG-Sweden | Gothenburg | SSI | production lot, |
| | | | Copenhagen |
| BCG-Phipps | Philadelphia | ATCC | # 35744 |
| BCG-Denmark | Danish 1331 | ATCC | # 35733 |
| BCG-Copenhagen | | ATCC | #27290 |
| BCG-Copenhagen | | IAF | dated 1961 |
| BCG-Tice | Chicago | vaccine | dated 1973 |
| BCG-Tice | Chicago | ATCC | # 35743 |
| BCG-Frappier | Montreal | IAF | primary lot, 1973 |
| BCG-Frappier, | INH-Montreal-R | IAF | primary lot, 1973 |
| resistant | | | |
| BCG-Frappier | Montreal | IAF | passage 946 |
| BCG-Connaught | Toronto | CL | bladder cancer treatment |
| BCG-Birkhaug | | ATCC | # 35731 |
| BCG-Prague | Czech | SSI | lyophilized 1968 |
| BCG-Glaxo | | vaccine | dated 1973 |
| BCG-Glaxo | | ATCC | # 35741 |
| BCG-Pasteur | | IAF | passage 888 |
| BCG-Pasteur | | IAF | dated 1961 |
| BCG-Pasteur | | IP | 1173P2-B |
| BCG-Pasteur | | IP | 1173P2-C |
| BCG-Pasteur | | IP | clinical isolate # 1 |
| BCG-Pasteur | | IP | clinical isolate # 2 |
| BCG-Pasteur | | ATCC | # 35734 |

Abbreviations:
IP = Institut Pasteur, Paris, France;
IAF = Institut Armand Frappier, Laval, Canada;
ATCC = American Type Culture Collection, Rockville, Md, USA;
SSI = Statens Serum Institute, Copenhagen, Denmark;
CL = Connaught Laboratories, Willowdale, Canada,
JATA = Japanese Anti-Tuberculosis Association;
INH = isoniazid. Canadian BCG's refers to BCG-Montreal and BCG-Toronto, the latter being derived from the former.

In performing the initial screening method, genomic DNA is isolated from two mycobacteria microbial cell cultures. The two DNA preparations are labeled, where a different label is used for the first and second microbial cultures, typically using nucleotides conjugated to a fluorochrome that emits at a wavelength substantially different from that of the fluorochrome tagged nucleotides used to label the selected probe. The strains used were the reference strain of Mycobacterium tuberculosis (H37Rv), other M. tb. laboratory strains, such as H37Ra, the O strain, M. tb. clinical isolates, the reference strain of Mycobacterium bovis, and different strains of Mycobacterium bovis BCG.

The two DNA preparations are mixed, and competitive hybridization is carried out to a microarray representing all of the open reading frames in the genome of the test microbe, usually H37Rv. Hybridization of the labeled sequences is accomplished according to methods well known in the art. In a preferred embodiment, the two probes are combined to provide for a competitive hybridization to a single microarray. Hybridization can be carried out under conditions varying in stringency, preferably under conditions of high stringency (e.g., 4×SSC, 10% SDS, 65° C.) to allow for hybridization of complementary sequences having extensive homology (e.g., having at least 85% sequence identity, preferably at least 90% sequence identity, more preferably having at least 95% sequence identity). Where the target sequences are native sequences the hybridization is preferably carried out under conditions that allow hybridization of only highly homologous sequences (e.g., at least 95% to 100% sequence identity).

Two color fluorescent hybridization is utilized to assay the representation of the unselected library in relation to the selected library (i.e., to detect hybridization of the unselected probe relative to the selected probe). From the ratio of one color to the other, for any particular array element, the relative abundance of that sequence in the unselected and selected libraries can be determined. In addition, comparison of the hybridization of the selected and unselected probes provides an internal control for the assay. An absence of signal from the reference strain, as compared to H37Rv, is indicative that the open reading frame is deleted in the test strain. The deletion may be further mapped by Southern blot analysis, and by sequencing the regions flanking the deletion.

Microarrays can be scanned to detect hybridization of the selected and the unselected sequences using a custom built scanning laser microscope as described in Shalon et al., Genome Res. 6:639 (1996). A separate scan, using the appropriate excitation line, is performed for each of the two fluorophores used. The digital images generated from the scan are then combined for subsequent analysis. For any particular array element, the ratio of the fluorescent signal from the amplified selected cell population DNA is compared to the fluorescent signal from the unselected cell population DNA, and the relative abundance of that sequence in the selected and unselected library determined.

Nucleic Acid Compositions

As used herein, the term “deletion marker”, or “marker” is used to refer to those sequences of M. tuberculosis complex genomes that are deleted in one or more of the strains or species, as indicated in Table 1. The bacteria of the M. tuberculosis complex include M. tuberculosis, M. bovis, and BCG, inclusive of varied isolates and strains within each species. Nucleic acids of interest include all or a portion of the deleted region, particularly complete open reading frames, hybridization primers, promoter regions, etc.

The term “junction” or “deletion junction” is used to refer to nucleic acids that comprise the regions on both the 3′ and the 5′ sequence immediately flanking the deletion. Such junction sequences are preferably used as short primers, e.g. from about 15 nt to about 30 nt, that specifically hybridize to the junction, but not to a nucleic acid comprising the undeleted genomic sequence. For example, the deletion found in M. bovis, at Rv0221, corresponds to the nucleotide sequence of the M. tuberculosis H37Rv genome, segment 12: 17432,19335. The junction comprises the regions upstream of position 17342, and downstream of 19335, e.g. a nucleic acid of 20 nucleotides comprising the sequence from H37Rv 17332-17342 joined to 19335-19345.

Typically, such nucleic acids comprising a junction will include at least about 7 nucleotides from each flanking region, i.e. from the 3′ and from the 5′ sequences adjacent to the deletion, and may be about 10 nucleotides from each flanking region, up to about 15 nucleotides, or more. Amplification primers that hybridize to the junction sequence, to the deleted sequence, and to the flanking non-deleted regions have a variety of uses, as detailed below.

The nucleic acid compositions of the subject invention encode all or a part of the deletion markers. Fragments may be obtained of the DNA sequence by chemically synthesizing oligonucleotides in accordance with conventional methods, by restriction enzyme digestion, by PCR amplification, etc. For the most part, DNA fragments will be at least about 25 nt in length, usually at least about 30 nt, more usually at least about 50 nt. For use in amplification reactions, such as PCR, a pair of primers will be used. The exact composition of the primer sequences is not critical to the invention, but for most applications the primers will hybridize to the subject sequence under stringent conditions, as known in the art. It is preferable to chose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

Usually, the DNA will be obtained substantially free of other nucleic acid sequences that do not include a deletion marker sequence or fragment thereof, generally being at least about 50%, usually at least about 90% pure and are typically “recombinant”, i.e. flanked by one or more nucleotides with which it is not normally associated on a naturally occurring chromosome.

For screening purposes, hybridization probes of one or more of the deletion sequences may be used in separate reactions or spatially separated on a solid phase matrix, or labeled such that they can be distinguished from each other. Assays may utilize nucleic acids that hybridize to one or more of the described deletions.

An array may include all or a subset of the deletion markers listed in Table 1. Usually such an array will include at least 2 different deletion marker sequences, i.e. deletions located at unique positions within the locus, and may include all of the provided deletion markers. Arrays of interest may further comprise other genetic sequences, particularly other sequences of interest for tuberculosis screening. The oligonucleotide sequence on the array will usually be at least about 12 nt in length, may be the length of the provided deletion marker sequences, or may extend into the flanking regions to generate fragments of 100 to 200 nt in length. For examples of arrays, see Ramsay (1998) Nat. Biotech. 16:40-44; Hacia et al. (1996) Nature Genetics 14:441-447; Lockhart et al. (1996) Nature Biotechnol. 14:1675-1680; and De Risi et al. (1996) Nature Genetics 14:457-460.

Nucleic acids may be naturally occurring, e.g. DNA or RNA, or may be synthetic analogs, as known in the art. Such analogs may be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The α-anomer of deoxyribose may be used, where the base is inverted with respect to the natural b-anomer. The 2′-OH of the ribose sugar may be altered to form 2′-O-methyl or 2′-O-allyl sugars, which provide resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Polypeptide Compositions

The specific deletion markers in Table 1 correspond to open reading frames of the M. tb genome, and therefore encode a polypeptide. The subject markers may be employed for synthesis of a complete protein, or polypeptide fragments thereof, particularly fragments corresponding to functional domains; binding sites; etc.; and including fusions of the subject polypeptides to other proteins or parts thereof. For expression, an expression cassette may be employed, providing for a transcriptional and translational initiation region, which may be inducible or constitutive, where the coding region is operably linked under the transcriptional control of the transcriptional initiation region, and a transcriptional and translational termination region. Various transcriptional initiation regions may be employed that are functional in the expression host.

In the present specification and claims, the term “polypeptide fragments”, or variants thereof, denotes both short peptides with a length of at least two amino acid residues and at most 10 amino acid residues, oligopeptides with a length of at least 11 amino acid residues, 20 amino acid residues, 50 amino acid residues, and up to about 100 amino acid residues; and longer peptides of greater than 100 amino acid residues up to the complete length of the native polypeptide.

The term substantially pure polypeptide fragment means a polypeptide preparation which contains at most 5% by weight of other polypeptide material with which it is natively associated, and lower percentages are preferred, e.g. at most 4%, at most 3%, at most 2%, at most 1%, and at most 0.5%. It is preferred that the substantially pure polypeptide is at least 96% pure, i.e. that the polypeptide constitutes at least 96% by weight of total polypeptide material present in the preparation, and higher percentages are preferred, such as at least 97%, at least 98%, at least 99%, at least 99.25%, at least 99.5%, and at least 99.75%. It is especially preferred that the polypeptide fragment is essentially free of any other antigen with which it is natively associated, i.e. free of any other antigen from bacteria belonging to the tuberculosis complex. This can be accomplished by preparing the polypeptide fragment by means of recombinant methods in a non-mycobacterial host, or by synthesizing the polypeptide fragment by the well-known methods of solid or liquid phase peptide synthesis, e.g. by the method described by Merrifield or variations thereof.

The M. tuberculosis polypeptide antigens provided herein include variants that are encoded by DNA sequences that are substantially homologous to one or more of the DNA sequences specifically recited herein, for example variants having at least 80%, 85%, 90%, 91%, 92%, 93%, 94%, 95%, 96%, 97%, 98%, 99%, or 99.5% sequence identity.

In a preferred embodiment of the invention, polypeptide fragments provide for an epitope of the deletion marker. The binding site of antibodies typically utilizes multiple non-covalent interactions to achieve high affinity binding. While a few contact residues of the antigen may be brought into close proximity to the binding pocket, other parts of the antigen molecule can also be required for maintaining a conformation that permits binding. The portion of the antigen bound by the antibody is referred to as an epitope. As used herein, an epitope is that portion of the antigen that is sufficient for high affinity binding. In a polypeptide antigen, generally a linear epitope will be at least about 7 amino acids in length, and may be at least 8, at least 9, at least 10, at least 11, at least 12, at least 14, at least 16, at least 18, at least 20, at least 22, at least 24,or at least 30 amino acid residues in length. However, antibodies may also recognize conformational determinants formed by non-contiguous residues on an antigen, and an epitope can therefore require a larger fragment of the antigen to be present for binding, e.g. a domain, or up to substantially all of a protein sequence. For each antigen there exists a plurality of epitopes that, in sum, represent the immunologic determinants of that antigen, although there are instances in which an antigen contains a single epitope.

The level of affinity of antibody binding that is considered to be “specific” will be determined in part by the class of antibody, e.g. antigen specific antibodies of the IgM class may have a lower affinity than antibodies of, for example, the IgG classes. As used herein, in order to consider an antibody interaction to be “specific”, the affinity will be at least about 10⁻⁷ M, usually about 10^{-8 to -9} M, and may be up to 10⁻¹¹ or higher for the epitope of interest. It will be understood by those of skill in the art that the term “specificity” refers to such a high affinity binding, and is not intended to mean that the antibody cannot bind to other molecules as well. One may find cross-reactivity with different epitopes, due, e.g. to a relatedness of antigen sequence or structure, or to the structure of the antibody binding pocket itself. Antibodies demonstrating such cross-reactivity are still considered specific for the purposes of the present invention.

Polypeptide sequences include analogs and variants produced by recombinant methods wherein such nucleic acids and polypeptide sequences are modified by substitution, insertion, addition, and/or deletion of one or more nucleotides in the nucleic acid sequence to cause the substitution, insertion, addition, and/or deletion of one or more amino acid residues in the recombinant polypeptide.

The polypeptides may be expressed in prokaryotes or eukaryotes in accordance with conventional ways, depending upon the purpose for expression. For large scale production of the protein, a unicellular organism, such as E. coli, B. subtilis, S. cerevisiae, or cells of a higher organism such as vertebrates, particularly mammals, e.g. COS 7 cells, may be used as the expression host cells. Small peptides can also be synthesized in the laboratory.

With the availability of the polypeptides in large amounts, by employing an expression host, the polypeptides may be isolated and purified in accordance with conventional ways. A lysate may be prepared of the expression host and the lysate purified using HPLC, exclusion chromatography, gel electrophoresis, affinity chromatography, or other purification technique. The purified polypeptide will generally be at least about 80% pure, preferably at least about 90% pure, and may be up to and including 100% pure. Pure is intended to mean free of other proteins, as well as cellular debris.

The polypeptide is used for the production of antibodies, where short fragments provide for antibodies specific for the particular polypeptide, and larger fragments or the entire protein allow for the production of antibodies over the surface of the polypeptide. Antibodies may be raised to isolated peptides corresponding to particular domains, or to the native protein.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage “display” libraries, usually in conjunction with in vitro affinity maturation.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Diagnostic Applications

Immunization with BCG typically leads to a positive response against tuberculin antigens in a skin test. In people who have been immunized, which includes a significant proportion of the world population, it is therefore difficult to determine whether a positive test is the result of an immune reaction to the BCG vaccine, or to an ongoing M. tb. infection. The subject invention has provided a number of open reading frame sequences that are present in M. tb isolates, but are absent in BCG. As a primary or a secondary screening method, one may test for immunoreactivity of the patient with the polypeptides encoded by such deletion markers. Diagnosis may be performed by a number of methods. The different methods all determine the presence of an immune response to the polypeptide in a patient, where a positive response is indicative of an M. tb infection. The immune response may be determined by determination of antibody binding, or by the presence of a response to intradermal challenge with the polypeptide.

Conventional tuberculosis diagnosis is carried out by examination of sputum by staining or culturing; chest x-rays or intradermal skin testing with a purified protein derivative of M. tuberculosis (PPD). All these methods either lack sensitivity or are time-consuming. An alternative is PCR based diagnostic which is a sensitive and fast approach, however, this method requires samples containing the M.tuberculosis bacterium which are not easily obtained, and the method also requires relatively expensive equipment and active enzymes. PPD based diagnostic tests are limited by the fact that most of the proteins in these preparations are shared among mycobacterial species, making specific diagnosis difficult in both persons infected with atypical mycobacterium and persons which have been BCG vaccianted. Intradermal skin testing is complicated by requiring two visits to the field station/physician: first to administer PPD (or single antigens) intradermally, and 3 days later to read the reaction.

As an alternative, the use of the Cell Mediated Immunology (CMI) method as an antigen-specific in vitro IFN-γ assay has shown promise for detection of mycobacterial infections. The assay is based on the detection of gamma interferon (IFN-γ) released specifically from whole blood cultures after stimulation with single antigens. The IFN-γ release assay is very sensitive, but it takes several days before a result is obtained and it is not simple to perform. The IFN-γ release assay requires the presence of viable cells and the tests therefore have to be performed within 12 hours from the time the blood sample is taken, and in this period the sample is preferably kept at 37° C.

In one method, a dose of the deletion marker polypeptide, formulated as a cocktail of proteins or as individual protein species, in a suitable medium is injected subcutaneously into the patient. The dose will usually be at least about 0.05 μg of protein, and usually not more than about 5 μg of protein. A control comprising medium alone, or an unrelated protein will be injected nearby at the same time. The site of injection is examined after a period of time for the presence of a wheal. The wheal at the site of polypeptide injection is compared to that at the site of the control injection, usually by measuring the size of the wheal. The skin test readings may be assessed by a variety of objective grading systems. A positive result will show an increased diameter at the site of polypeptide injection as compared to the control, usually at least about 50% increase in size, more usually at least 100% increase in size.

Proliferation assays measure the level of T cell proliferation in response to a specific antigen, and are widely used in the art. In an exemplary assay, recipient lymph node, blood or spleen cells are obtained at one or more time points after transplantation. A suspension of from about 10⁴ to 10⁷ cells, usually from about 10⁵ to 10⁶ cells is prepared and washed, then cultured in the presence of a control antigen, and test antigens. The cells are usually cultured for several days. Antigen-induced proliferation is assessed by the monitoring the synthesis of DNA by the cultures, e.g. incorporation of ³H-thymidine during the last 18 H of culture. T cells may be isolated from patient peripheral blood, lymph nodes, or from the site of disease lesions. Reactivity assays may be performed on primary T cells, or the cells may be fused to generate hybridomas.

An alternative cell mediated immunoassay measures the induction of an in vitro recall response determined by release of γlFN, e.g. of at least 500 μg/ml, usually 1,000 μg/ml from PBMC or whole blood drawn from a suspected TB patient. The induction is performed by the addition of a deletion antigen or plurality of deletion antigens to a suspension comprising PBMC or whole blood cells. The release of γIFN may be quantitated by determination of the γIFN present in supernatants harvested after at least about 5 days following addition of the antigen to the cells.

Serologic Assays

Serological tests for TB infections based on the presence of antibodies in a blood sample have been rejected by the scientific community many years ago, as the antigens tested were not able to discriminate TB patients from healthy controls, and the sensitivity was very low (Pottumarthy et al. (2000) J Clin Microbiol 38(6):2227-31). However, in the present invention, antigens that are specific for M. tuberculosis are used, and preferably combinations of antigens specific for M. tuberculosis are used. Advantages of using a serological assay are that a test based on monitoring the presence of specific antibodies in TB patients blood is cheap, simple and fast to perform. Most laboratories around the world have the equipment and the expertise needed and the test format is very simple and robust.

A further advantage of serologic tests is in TB diagnosis of HIV infected patients. TB is a HIV-related opportunistic infection, and in some populations up to 30% of individuals that have pulmonary TB are also HIV-positive. The TB diagnosis of the HIV infected TB patients are complicated by a decreased cell-mediated response to M. tuberculosis antigens which dramatically lowers the sensitivity of a skin test or a IFN-γ release assay for diagnosing TB. For this group of patients, a TB diagnosis based on detecting antibodies is therefore an extremely attractive approach. Other very important patient groups are patients on immunosuppressive drugs and patients with advance disease whom in many cases will not respond to any CMI based assay. However, for these patient groups it will still be possible to monitor for the presence of specific antibodies in the blood.

It is well known that the antibody repertoire of TB patients is heterogeneous, and it is therefore not likely that all patients will recognize the same mycobacterial antigen, as is also demonstrated by the following examples. It is therefore preferable for serological methods and kit for the practice of such methods to comprise a plurality of deletion antigens.

Serologic assays determine whether a patient sample, e.g. blood, lymph, saliva, etc., contains antibodies that specifically bind to deletion antigens, where the presence of such antibodies is indicative of prior, or current, infection with a virulent strain of the M. tuberculosis complex. Antigens of interest for such assays include, without limitation, combinations of one or more of Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76), which are optionally combined with r38 kDa protein. Assays may use 2 of the deletion antigens, 3 of the deletion antigens, 4 of the deletion antigens, 5 of the deletion antigens, 6 of the deletion antigens, 7 of the deletion antigens, 8 of the deletion antigens, 9 of the deletion antigens, or more. Preferred antigens are not cross-reactive with environmental mycobacteria antigens, e.g. with M. avium antigens. A preferred diagnostic reagent will provide high sensitivity.

In one embodiment of the invention, a plurality of antigens from the provided genetic markers is used in the diagnosis of M. tuberculosis infection. One or more antigenic polypeptides or fragments thereof encoded by the provided deletion markers, herein collectively referred to as “antigens” or “deletion antigens” can be used in diagnostic assays. The antigens may be combined in a single test format, e.g. well, tube, etc., for use in the analysis of binding or immunoreactivity with a patient sample. Alternatively the antigens are assayed in separate test wells, tubes, etc., and the results collated or compiled prior to diagnosis. Antigens can be separately synthesized, e.g. by recombinant or chemical methods, or epitopes from different markers can be combined and produced in a fusion protein.

Biological samples from which patient antibodies may be collected include blood and derivatives therefrom, e.g. serum, plasma, fractions of plasma, etc. Other sources of samples are body fluids such as synovial fluid, lymph, cerebrospinal fluid, bronchial aspirates, and may further include saliva, milk, urine, and the like. Antibodies may also be obtained from B lymphocytes, which may be collected from blood, tissues such as spleen, thymus, lymph nodes, etc. The lymphocytes may be analyzed intact, or lysates may be prepared for analysis.

Methods of diagnosis may use in vitro detection of specific binding between antibodies in a patient sample and the subject polypeptides, either as a cocktail or as individual protein species, where the presence of specific binding is indicative of a prior, or current, infection. Measuring the concentration of polypeptide specific antibodies in a sample or fraction thereof may be accomplished by a variety of specific assays. In general, the assay will measure the reactivity between a patient sample, usually blood derived, generally in the form of plasma or serum. The patient sample may be used directly, or diluted as appropriate, usually about 1:10 and usually not more than about 1:10,000. Immunoassays may be performed in any physiological buffer, e.g. PBS, normal saline, HBSS, dPBS, etc.

For example, diagnosis may utilize an ELISA technique or western blot, where a serum sample is diluted in PBS or other acceptable excipient, and incubated with the deletion antigen, where a positive result in the ELISA or a visual response in a western blot is indicative of reactivity.

In a preferred embodiment, a conventional sandwich type assay is used. A sandwich assay is performed by first attaching the polypeptide to an insoluble surface or support. The polypeptide may be bound to the surface by any convenient means, depending upon the nature of the surface, either directly or through specific antibodies. The particular manner of binding is not crucial so long as it is compatible with the reagents and overall methods of the invention. They may be bound to the plates covalently or non-covalently, preferably non-covalently. Samples, fractions or aliquots thereof are then added to separately assayable supports (for example, separate wells of a microtiter plate) containing support-bound polypeptide. Preferably, a series of standards, containing known concentrations of antibodies is assayed in parallel with the samples or aliquots thereof to serve as controls.

Immune specific receptors may be labeled to facilitate direct, or indirect quantification of binding. Examples of labels which permit direct measurement of second receptor binding include radiolabels, such as ³H or ¹²⁵I, fluorescers, dyes, beads, chemilumninescers, colloidal particles, and the like. Examples of labels which permit indirect measurement of binding include enzymes where the substrate may provide for a colored or fluorescent product. In a preferred embodiment, the second receptors are antibodies labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In some cases, a competitive assay, will be used. In addition to the patient sample, a competitor to the antibody is added to the reaction mix. The competitor and the antibody compete for binding to the polypeptide. Usually, the competitor molecule will be labeled and detected as previously described, where the amount of competitor binding will be proportional to the amount of Immune present. The concentration of competitor molecule will be from about 10 times the maximum anticipated Immune concentration to about equal concentration in order to make the most sensitive and linear range of detection.

Alternatively, antibodies may be used for direct determination of the presence of the deletion marker polypeptide. Antibodies specific for the subject deletion markers as previously described may be used in screening immunoassays. Samples, as used herein, include microbial cultures, biological fluids such as tracheal lavage, blood, etc. Also included in the term are derivatives and fractions of such fluids. Diagnosis may be performed by a number of methods. The different methods all determine the absence or presence of polypeptides encoded by the subject deletion markers. For example, detection may utilize staining of mycobacterial cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including microscopy, radiography, scintillation counting, etc.

An alternative method for diagnosis depends on the in vitro detection of binding between antibodies and the subject polypeptides in solution, e.g. a cell lysate. Measuring the concentration of binding in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently. The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Samples are then added to separately assayable supports (for example, separate wells of a microtiter plate) containing antibodies. Preferably, a series of standards, containing known concentrations of the polypeptides is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing a second antibody is applied. The antibody will bind with sufficient specificity such that it can be distinguished from other components present. The second antibodies may be labeled to facilitate direct, or indirect quantification of binding. Examples of labels that permit direct measurement of second receptor binding include radiolabels, such as ³H or ¹²⁵I, fluorescers, dyes, beads, chemilumninescers, colloidal particles, and the like. Examples of labels which permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the polypeptide, conveniently using a labeling method as described for the sandwich assay.

The agents utilized in the methods of the invention may be provided in a kit, which kit may further include instructions for use. Such a kit will comprise one or more deletion antigens, usually in a dose and form suitable for analytical methods. The kit may further comprise reagents, such as positive and negative controls, second stage antibodies for detection, where the provided antibodies may be conjugated to an enzyme for detection, substrates for such enzymes, and plates, buffers and the like for performing assays. Preferably a plurality of deletion antigens are included in a kit, as described above. It is advantageous to combine two or more serological antigens selected from the group consisting of Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76), which are optionally combined with r38 kDa protein, or fragments thereof, usually epitopic fragments thereof, in a kit. The kit may further comprise the r38 kDa protein or a fragment thereof.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

Experimental

### EXAMPLE 1

Methods:

The technical methods used begin with extraction of whole genomic DNA from bacteria grown in culture.

Day 1

Inoculate culture medium of choice (LJ/7H9) and incubate at 35° C. until abundant growth. Dispense 500 μl 1×TE into each tube. (If DNA is in liquid medium, no TE needed.) Transfer loopful (sediment) of cells into microcentrifuge tube containing 500 μl of 1*TE. If taking DNA from liquid medium, let cells collect in bottom of flask. Pipette cells (about 1 ml) into tube. Heat 20 min at 80° C. to kill cells, centrifuge, resuspend in 500 μl of 1*TE. Add 50 μl of 10 mg/ml lysozyme, vortex, incubate overnight at 37° C.

Day 2

Add 70 μl of 10% SDS and 10 μl proteinase K, vortex and incubate 20 min. at 65° C. Add 100 μl of 5M NaCl. Add 100 μl of CTAB/NaCl solution, prewarmed at 65° C. Vortex until liquid content white (“milky”). Incubate 10 min at 65° C. Outside of hood, prepare new microcentrifuge tubes labeled with culture # on top, and culture #, tube #, date on side. Add 550 μl isopropanol to each and cap. Back in the hood, add 750 μl of chloroform/isoamyl alcohol, vortex for 10 sec. Centrifuge at room temp for 5 min. at 12,000 g. Transfer aqueous supernatant in 180 μl amounts to new tube using pipetter, being careful to leave behind solids and non-aqueous liquid. Place 30 min at −20 C. Spin 15 min at room temp in a microcentrifuge at 12,000 g. Discard supernatant; leave about 20 μl above pellet. Add 1 ml cold 70% ethanol and turn tube a few times upside down. Spin 5 min at room temp in a microcentrifuge. Discard supernatant; leave about 20 μl above the pellet. Spin 1 min in a microcentrifuge and discard cautiously the last 20 μl supernatant just above the pellet using a pipetter (P-20). Be sure that all traces of ethanol are removed. Allow pellet to dry at room temp for 10 min or speed vac 2-3 min. (Place open tubes in speed vac, close lid, start rotor, turn on vacuum. After 3 min. push red button, turn off vacuum, turn off rotor. Check if pellets are dry by flicking tube to see if pellet comes away from side of tube.) Redissolve the pellet in 20-50 μl of ddH20. Small pellets get 20, regular sized get 30 and very large get 50. DNA can be stored at 4° C. for further use.

DNA array: was made by spotting DNA fragments onto glass microscope slides which were pretreated with poly-L-lysine. Spotting onto the array was accomplished by a robotic arrayer. The DNA was cross-linked to the glass by ultraviolet irradiation, and the free poly-L-lysine groups were blocked by treatment with 0.05% succinic anhydride, 50% 1-methyl-2-pyrrolidinone and 50% borate buffer.

The majority of spots on the array were PCR-derived products, produced by selecting over 9000 primer pairs designed to amplify the predicted open reading frames of the sequences strain H37Rv (ftp.sanger.ac.uk/pub/TB.seq). Some internal standards and negative control spots including plasmid vectors and non-M.tb. DNA were also on the array.

Therefore, with the preparation for an array that contained the whole genome of Mycobacterium tuberculosis, we compared BCG-Connaught to Mycobacterium tuberculosis, using the array for competitive hybridization. The protocol follows:

DNA labeling protocol. Add 4 μg DNA in 20 μl H₂O, 2 ml dN10N6 and 36 μl H₂O. 2 ml DNA spike for each DNA sample, for total of 60 μl. Boil 3 minutes to denature DNA, then snap cool on ice water bath. Add 1 μl dNTP (5 mM ACG), 10 μl 10 buffer, 4 μl Klenow, 22 μl H₂O to each tube. Add 3 μl of Cy3 or Cy5 dUTP, for total of 100 μl. Incubate 3 hours at 37 C. Add 11 μl 3M NaAc, 250 μl 100% EtOH to precipitate, store O/N at −20 C. Centrifuge genomic samples 30 minutes at 13K to pellet precipitate. Discard supernatant, add 70% EtOH, spin 15 minutes, discard sup and speed-vac to dry. This provides DNA for two experiments.

DNA hybridization to microarray. protocol. Resuspend the labeled DNA in 11 μl dH₂O (for 2 arrays). Run out 1 μl DNA on a 1.5% agarose gel to document sample to be hybridized. Of the remaining 10 μl of solution, half will be used for this hyb, and half will be left for later date. Take 5 μl of solution Cy3 and add to same amount of Cy5 solution, for total volume 10 μl mixed labeled DNA. Add 1 μl tRNA, 2.75 μl 20×SSC, 0.4 μl SDS, for total volume 14.1 μl. Place on slide at array site, cover with 22 mm coverslip, put slide glass over and squeeze onto rubber devices, then hybridize 4 hours at 65 C. After 4 hours, remove array slides from devices, leave coverslip on, and dip in slide tray into wash buffer consisting of 1×SSC with 0.05% SDS for about 2 minutes. Cover slip should fall off into bath. After 2 minutes in wash buffer, dip once into a bath with 0.06×SSC, then rinse again in 0.06×SSC in separate bath. Dry slides in centrifuge about 600 rpm. They are now ready for scanning.

Fluorescence scanning and data acquisition. Fluorescence scanning was set for 20 microns/pixel and two readings were taken per pixel. Data for channel 1 was set to collect fluorescence from Cy3 with excitation at 520 nm and emission at 550-600 nm. Channel 2 collected signals excited at 647 nm and emitted at 660-705 nm, appropriate for Cy5. No neutral density filters were applied to the signal from either channel, and the photomultiplier tube gain was set to 5. Fine adjustments were then made to the photomultiplier gain so that signals collected from the two spots containing genomic DNA were equivalent.

To analyze the signal from each spot on the array, a 14×14 grid of boxes was applied to the data collected from the array such that signals from within each box were integrated and a value was assigned to the corresponding spot. A background value was obtained for each spot by integrating the signals measured 2 pixels outside the perimeter of the corresponding box. The signal and background values for each spot were imported into a spreadsheet program for further analysis. The background values were subtracted from the signals and a factor of 1.025 was applied to each value in channel 2 to normalize the data with respect to the signals from the genomic DNA spots.

Because the two samples are labeled with different fluorescent dyes, it is possible to determine that a spot of DNA on the array has hybridized to Mycobacterium tuberculosis (green dye) and not to BCG (red dye), thus demonstrating a likely deletion from the BCG genome.

However, because the array now contains spots representing 4000 spots, one may expect up to 100 spots with hybridization two standard deviations above or below the mean. Consequently, we have devised a screening protocol, where we look for mismatched hybridization in two consecutive genes on the genome. Therefore, we are essentially looking only for deletions of multiple genes at this point.

To confirm that a gene or group of genes is deleted, we perform Southern hybridization, employing a separate probe from the DNA on the array. Digestions of different mycobacterium DNAs are run on an agarose gel, and transferred to membranes. The membranes can be repeatedly used for probing for different DNA sequences. For the purposes of this project, we include DNA from the reference strain of Mycobacterium tuberculosis (H37Rv), from other laboratory strains, such as H37Ra, the O strain, from clinical isolates, from the reference strain of Mycobacterium bovis, and from different strains of Mycobacterium bovis BCG.

Once a deletion is confirmed by Southern hybridization, we then set out to characterize the exact genomic location. This is done by using polymerase chain reaction, with primers designed to be close to the edges of the deletion, see Talbot (1997) J Clin Micro. 35: 566-9

Primers have been chosen to amplify across the deleted region. Only in the absence of this region does one obtain an amplicon. PCR products were examined by electrophoresis (1.5% agarose) and ethidium bromide staining.

Once a short amplicon is obtained, this amplicon is then sequenced. A search of the genome database is performed to determine whether the sequence is exactly identical to one part of the Mycobacterium tuberculosis genome, and that the next part of the amplicon is exactly identical to another part of the Mycobacterium tuberculosis genome. This permits precise identification of the site of deletion.

Below follows an example of the kind of report obtained:

rd6 bridging PCR, blast search of sequence

emb|Z79701|MTCY277 Mycobacterium tuberculosis cosmid Y277

Length=38,908

Plus Strand HSPs:

Score=643 (177.7 bits), Expect=1.6e-54, Sum P(2)=1.6e-54

Identities=129/131 (98%), Positives=129/131 (98%), Strand=Plus/Plus

| | | | | |
|---|---|---|---|---|
| Query: | 12 | ANTAGTAATGTGCGAGCTGAGCGATGTCGCCGCTCCCAAAAATTACCAATGGTTNGGTCA | 71 | (SEQ ID NO:130) |
| | | \| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\| | | |
| Sbjct: | 24784 | AGTAGTAATGTGCGAGCTGAGCGATGTCGCCGCTCCCAAAAATTACCAATGGTTTGGTCA | | SEQ ID NO:131) |
| Query: | 72 | TGACGCCTTCCTAACCAGAATTGTGAATTCATACAAGCCGTAGTCGTGCAGAAGCGCAAC | | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 24844 | TGACGCCTTCCTAACCAGAATTGTGAATTCATACAAGCCGTAGTCGTGCAGAAGCGCAAC | | |
| Query: | 132 | ACTCTTGGAGT 142 | | |
| | | \|\|\|\|\|\|\|\|\|\|\| | | |
| Sbjct: | 24904 | ACTCTTGGAGT 24914 | | |

Score=224 (61.9 bits), Expect=1.6e-54, Sum P(2)=1.6e-54

Identities=46/49 (93%), Positives=46/49 (93%), Strand=Plus/Plus

| | | | | |
|---|---|---|---|---|
| Query: | 141 | GTGGCCTACAACGGNGCTCTCCGNGGCGCGGGCGTACCGGATATCTTAG | 189 | (SEQ ID NO:132) |
| | | \| \|\|\|\|\|\|\|\|\|\|\|\| \|\|\|\|\|\|\|\| \|\|\|\|\|\|\|\|\|\|\|\|\|\|\|\|\|\|\|\|\|\|\|\|\| | | |
| Sbjct: | 37645 | GCGGCCTACAACGGCGCTCTCCGCGGCGCGGGCGTACCGGATATCTTAG | 37693 | (SEQ ID NO:133) |

This process is repeated with each suggested deletion, beginning with the three previously described deletions to serve as controls. Sixteen deletions have been identified by these methods, and are listed in Table 1.

### EXAMPLE 2

Tuberculosis Serodiagnostic Assays

The following example uses a serodiagnostic: a non-invasive ELISA based test. The methods can be used to test large populations and thereby identifying patients before they present with clinical symptoms, as well as to identify patients with active TB.

Immune-based specific diagnosis of tuberculosis is complicated by the common use of M. bovis BCG vaccines. One way around this problem is to use proteins only expressed in M. tuberculosis. The comparison of the M. tuberculosis H37Rv genome with genomes from different M. bovis BCG vaccine strains identified 12 different regions that are not present in any of the M. bovis BCG vaccine strains tested, as described above. These unique regions encode 100 ORFs, however, even though these ORFs are not present in BCG they may be present in environmental mycobacterium and therefore process a potential specificity problem. The 100 ORFs present in M. tuberculosis but deleted in M. bovis were compared with the genome for the environmental mycobacterium M. avium, and the ORFs with no homology to any ORF predicted in the M. avium genome were selected. 56 M. tuberculosis ORFs were selected, and were expressed as recombinant proteins in E. coli. By comparing the presence of M. tuberculosis specific antibodies in blood samples from both TB patients and healthy controls we found that nine antigens were able to discriminated between M. tuberculosis infected persons in a serological assay with a sensitivity of the single antigens on more that 38% and a specificity of more than 95%.

General Methods

Strains, growth media and plasmids. Escherichia coli strain DH5α was used for all cloning steps and E. coli strain BL21 SI was used for protein expression. Luria-Bertani medium with 100 mg/L ampicillin was used in plates and cultures for E. coli strain DH5α whereas BL21 SI clones were grown in 10 g/L yeast extract and 5 g/L peptone (LB(-NaCl)) with 100 mg/L ampicillin. The expression vector pDest17 (Invitrogen) was used in this study. It adds an N-terminal Histidine-tag to the recombinant protein.

PCR, primer design, and cloning. Oligonucleotides used for PCR amplification were designed with recombination sites for cloning into pDest17 via pDONR201 (Invitrogen). The gene-specific part of each oligonucleotide was determined by using the Primer Express software (PE Applied Biosystems) using standard settings and an annealing temperature of 60° C. Primer sequences are given in table 3. All PCR reactions were conducted in 50 μL using Taq polymerase (Platinum Taq, Invitrogen). Primer concentrations were 0.4 μM and the DNA source was chromosomal M. tuberculosis H37Rv DNA. The PCR reactions were initiated by a 3 min 95° C. denaturation followed by 30-35 cycles of denaturation (95° C., 30 s), annealing (55° C., 30 s) and extension (72° C., 1 min/kb), and a final extension (72° C., 10 min). The manufacturer's protocol was followed for recombinational cloning (Invitrogen).

TABLE 3

Sequences of the gene specific parts of the deoxynucleotide oligos
used for cloning into expression vector pDEST17.

| | | |
|---|---|---|
| Rv number | Forward primer¹5′→3′ | Reverse primer²5′→3′ |

| | | |
|---|---|---|
| Rv1516 | SEQ ID NO:138 | SEQ ID NO:139 |
| | gtg ccg tac gtc cgc c | ggc tcg aca gcc gcg t |
| Rv1575 | SEQ ID NO:140 | SEQ ID NQ:141 |
| | gcg ccg ctg gcc gcc g | gat gtg ctg gtg cgc aac |
| Rv1586 | SEQ ID NO:142 | SEQ ID NO:143 |
| | gtg aga tac act aca cct gt | tcg cca att cac ctg cac c |
| Rv1966 | SEQ ID NO:144 | SEQ ID NO:145 |
| | aga cgc ggg ccg ggt | cta tgg ctg ctc ccc |
| Rv1976 | SEQ ID NO:146 | SEQ ID NO:147 |
| | cgg tgg att gtc gac | cta cgt gcg gcg ggc |
| Rv2660 | SEQ ID NO:148 | SEQ ID NO:149 |
| | gtg ata gcg ggc gtc ga | cta gtg aaa ctg gtt caa tcc cag ta |
| Rv3118 | SEQ ID NO:150 | SEQ ID NO:151 |
| | tgc tct gga ccc aag caa | cta ggt gat ctt gac gtc tac ctc gt |
| Rv3429 | SEQ ID NO:152 | SEQ ID NO:153 |
| | cat cca atg ata cca gcg ga | cta gta gat ctg cgg cgg ct |
| Rv3618 | SEQ ID NO:154 | SEQ ID NO:155 |
| | aag gca ccg ttg cgt ttt | cta gcc cgc ttc ccc ttg t |

¹Upstream recombination sequence SEQ ID NO:156 5′-gggg aca agt ttg tac aaa aaa gca ggc tta-3′
²Downstream recombination sequence SEQ ID NO:157 5′-gggg ac cac ttt gta caa gaa agc tgg gtc-3′

Recombinant protein expression and purification. Overnight cultures of recombinant E. coli BL21 SI strains were grown to mid-exponential phase and induced with 0.3 M NaCl for 4 h. Bacteria were harvested, resuspended in 1/50 of B-PER reagent (Pierce) to disrupt the outer membrane, washed twice in B-PER and resuspended again in 1/50 B-PER reagent. Lysozyme (200 mg/L) and DNase 1 (25 mg/L) were added and samples incubated end-over-end for 1 hour at room temp. Inclusion bodies were harvested by centrifugation and washed twice in 20 mM Tris-HCl pH 8, 0.1 M NaCl, 1 mM EDTA pH 8 and 0.1% deoxycholic acid. The final pellet was resuspended in wash buffer (4 M guanidine-HCl, 50 mM Na-phosphate, pH 7.2, 300 mM NaCl). Denaturated proteins were applied to metal affinity columns (Talon columns, Clontech) and washed with 3 column volumes of wash buffer. Recombinant proteins were eluted by adding 3 column volumes of the wash buffer supplemented with 150 mM imidazole. Flow trough, wash and eluates were collected, and selected fractions were pooled after examination on Coomassie blue-stained SDS-PAGE gels. All proteins purified this way were dialysed against an ammonium/CHAPS pH 9.5 buffer and protein concentration was measured (Pierce) using BSA as a standard.

Serological Recognition of the Selected Recombinant M. tuberculosis Proteins.

To test the potential of the proteins as serological antigens, sera were collected from 8 TB patients and 4 healthy BCG non-vaccinated controls and were assayed for antibodies recognizing the recombinantly produced proteins in an ELISA assay as follows: Each of the sera were absorbed with Promega E. coli extract (S3761) for 4 hours at room temperature, and the supernatants were collected after centrifugation. 0.5 μg/ml of the proteins in carbonate buffer (pH 9.6) were coated over night at 4° C. to a polystyrene plate (Maxisorp, Nunc). The plates were washed in PBS-0.05% Tween-20 and the sera applied in a dilution of 1:100. After 1 hour of incubation the plates were washed 3 times with PBS-0.05% Tween-20, and 100 ul per well of peroxidase-conjugated Rabbit Anti-Human IgA, IgG, IgM was applied in a dilution of 1:8000 to each well. After 1 hour of incubation the plates were washed 3 times with PBS-0.05% Tween-20. 100 ul of substrate (TMB PLUS, Kem-En-Tec) was added per well, the reaction was stopped after 30 min with 0.2 M Sulphuric acid, and the absorbance was read at 405 nm. The result for the nine best antigens are shown in table 4.

TABLE 4

| | Percent (n) positive | Percent (n) positive | |
|---|---|---|---|
| Protein | TB patients | healthy controls | Cut off |
| Rv1516 | 88 (7) | 0 (0) | 0.3 |
| Rv1575 | 25 (2) | 0 (0) | 0.3 |
| Rv1586 | 13 (1) | 0 (0) | 0.3 |
| Rv1966 | 88 (7) | 0 (0) | 0.2 |
| Rv1976 | 38 (3) | 0 (0) | 0.2 |
| Rv2660 | 88 (7) | 0 (0) | 0.2 |
| Rv3118 | 75 (6) | 0 (0) | 0.3 |
| Rv3429 | 63 (5) | 0 (0) | 0.3 |
| Rv3618 | 100 (8) | 0 (0) | 0.2 |
| r38 kDa | 75 (6) | 0 (0) | 0.2 |
| Serological recognition of the selected proteins by TB patients (n = 8) and healthy controls (n = 4). The percentage of responders as well as the number of persons responding in each group is indicated. For comparison, recombinant 38 kDa antigen (r38 kDa, Rv0934) was included in the panel of recombinant M. tuberculosis proteins investigated. | | | |
| # r38 kDa is considered a promising serological antigen (e.g. (4)). The cut-off values for positive responses are indicated in the table. | | | |

As shown in table 4, Rv1516, Rv1966, Rv2660, Rv3118, Rv3429, Rv3618, and r38kDa are recognized by ≧50% of the TB patients tested. In addition, Rv1516, Rv3118, and Rv3429 were recognized with high OD values (>0.7) by one or more of the TB patients, indicating a particular high amount of specific antibodies to these proteins. None of the proteins are recognized by healthy non-BCG vaccinated controls, which demonstrates the potential of these proteins to differentiate between M. tuberculosis infected individuals and healthy individuals. All these proteins are therefore promising diagnostic candidates.

Rv1516, Rv1586, Rv2660, Rv3118, RV3618 and Rv3429 are Serological Targets in TB Patients

Sera were collected from TB patients (all proven culture positive for M. tuberculosis), and 33 healthy controls. The sera were assayed for antibodies recognizing recombinant Rv1516, Rv1586, Rv2660, Rv3118, Rv3429, Rv3618 and r38 kDa as described above, except that each antigen was used in a concentration of 1 μg/ml. The results are shown in Table 5.

TABLE 5

| | TB patients | | | Healthy controls | | | |
|---|---|---|---|---|---|---|---|
| | Patients | Positive | Mean | Controls | Positive | Mean | Cut-off |
| r38 kDa | 42 | 7 (17%) | 0.38 | 33 | 0 (0%) | 0.22 | 0.53 |
| Rv1516 | 58 | 25 (43%) | 0.29 | 33 | 1 (3%) | 0.14 | 0.28 |
| Rv1586 | 58 | 35 (60%) | 0.55 | 33 | 0 (0%) | 0.32 | 0.53 |
| Rv2660 | 58 | 14 (24%) | 0.36 | 33 | 1 (3%) | 0.15 | 0.34 |
| Rv3118 | 58 | 13 (22%) | 0.96 | 33 | 1 (3%) | 0.59 | 1.17 |
| Rv3429 | 58 | 6 (10%) | 0.30 | 33 | 1 (3%) | 0.18 | 0.44 |
| Rv3618 | 56 | 20 (36%) | 0.47 | 30 | 1 (3%) | 0.28 | 0.48 |

Serological evaluation of 6 antigens tested in TB patients and healthy controls. Cut-off is defined as the mean value in the group of healthy controls plus 3 times standard deviations. The 42 donors used for evaluating r38 kDa are all included in the group of 58 donors used for evaluating the other antigens.

In table 5 the responses to Rv1516, Rv1586, Rv2660, Rv3118, Rv3618 and Rv3429 are compared with the response to r38 kDa in a larger panel of donors. All antigens gave a positive response in at least 6 donors, and the antigens Rv1516, Rv1586, Rv2660, Rv3618 and Rv3118 are recognized by more TB patients than the r38 kDa protein, demonstrating the potential of Rv1516, Rv1586, Rv2660, Rv3118, Rv3618 and Rv3429 as serological antigens for diagnosis of TB.

Sensitivity and Specificity of Selected Antigen Combinations.

It is well known that the antibody repertoire of TB patients is heterogeneous, and it is therefore not likely that all patients will recognize the same mycobacterial antigen, as also demonstrated by these results. It is therefore most likely that a serological kit for the diagnosis of M. tuberculosis infection will consist of more than one component, and in this respect it will advantageous to combine promising serological antigens like Rv1516, Rv1586, Rv2660, Rv3118 or Rv3429 with each other or with other promising, serological antigens such as the r38 kDa protein.

For a diagnostic TB reagent it is important to achieve a very high sensitivity and as none of the antigens alone detects all infected individuals a combination of antigens is necessary. The higher sensitivity by combining antigens is demonstrated in Table 6 (the results were achieved by combining the data provided above). In practice this can be accomplished either by mixing the antigens in the same well in the ELISA plate or by combining the results from multiple wells incubated with the same blood sample. Alternatively the proteins of interests can be produced as recombinant fusion proteins comprising of at least two proteins or B cell epitopes and the resulting fusion molecule can hereafter be used in the serological assays.

TABLE 6
Calculated sensitivity (sens.) and specificity (spec.)
of selected antigen combinations

| # | Antigens | Sens. | Spec. |
|---|---|---|---|
| 2 | Rv1586 + Rv3118 | 64% | 97% |
| 2 | Rv1586 + Rv3618 | 64% | 97% |
| 3 | Rv1586 + Rv3118 + Rv3618 | 70% | 93% |
| 4 | Rv1516c + Rv1586 + Rv3118 + RV3618 | 73% | 90% |
| 4 | Rv1586 + Rv1976 + Rv3118 + Rv3618 | 73% | 90% |
| 4 | Rv1586 + Rv2660c + Rv3118 + Rv3618 | 73% | 93% |

For the combinations shown in Table 6 it is advantageous to combine two, three, or four antigens, which will give a higher sensitivity than the single antigen and still a high specificity (90% or more). In particular the combination of Rv1586+Rv2660c+Rv3118+Rv3618 is very efficient in this study population. The combinations shown in table 6 are only examples and other useful combinations can be envisaged as antigens may be combined and lead to increased sensitivity. In addition, other antigens can be combined with the above-defined proteins, for example the 38 kDa antigen, which may be combined with any of the above described antigens and may increase the sensitivity.

It should be noted that it has been observed that different populations react to different antigens, and it may therefore be necessary to define different combinations for different populations. Therefore, combinations which does not give high sensitivity in the tested study population may be very efficient as diagnostic reagents when tested in another population.

Rv1516, Rv1575, Rv1586, Rv1976, Rv1966, Rv2660, Rv3118, Rv3618 and Rv3429 are Serological Targets in TB Patients from High Endemic Regions.

To demonstrate the potential of the antigens as serological targets in a population from a high endemic region sera from Uganda was obtained from the WHO sera bank from both HIV positive and HIV negative TB patients. The sera were assayed for antibodies recognizing recombinant Rv1516, Rv1575, Rv1586, Rv1976, Rv2660, Rv1966, Rv3118, Rv3429, Rv3618 and r38 kDa as described above. The results are shown in Table 7.

TABLE 7

| | | | Sensitivity | Sensitivity |
|---|---|---|---|---|
| Antigen | Cut-off | Specificity | HIV+ TB patients | HIV− TB patients |
| Rv1516 | 0.59 | 100% | 34% | 13% |
| Rv1575 | 1.98 | 100% | 4% | 15% |
| Rv1586 | 1.09 | 100% | 7% | 6% |
| Rv1976 | 0.67 | 100% | 7% | 13% |
| Rv2660 | 0.58 | 100% | 36% | 25% |
| Rv1966 | 0.24 | 100% | 59% | 75% |
| Rv3118 | 0.88 | 100% | 48% | 45% |
| Rv3429 | 0.2 | 100% | 85% | 91% |
| Rv3618 | 0.62 | 100% | 49% | 53% |
| 38 kDa | 0.99 | 100% | 22% | 27% |

Serological evaluation of ten antigens tested in HIV positive (n = 73) and HIV negative (n = 110) TB patients from a high endemic area. Cut-off is defined as the mean value in the group of healthy controls plus 3 times standard deviations.

In table 7 the responses of the antigens are compared with the response to r38 kDa. All antigens gave a positive response in at least 3 donors, and the antigens Rv1516, Rv1966, Rv3429, Rv3618 and Rv3118 are recognized by more than 30% of the TB patients in both the HIV positive and HIV negative donors (which is more than the r38 kDa protein). Furthermore, the antigens Rv1966, Rv3429 and Rv3618 are recognized by more than 50% of the HIV negative TB patients and Rv3429 is recognized by 85% of the HIV positive donors.

These data demonstrate the potential of the deletion antigens as serological antigens for diagnosis of TB for both HIV positive and HIV negative patients. In addition, combinations of the other antigens (which all are recognized by both HIV− and HIV+TB patients) may lead to a increased sensitivity and therefore cocktails antigens may be superior to the use of a single frequently recognized antigens.

References

1: Pottumarthy S, Wells V C, Morris A J.: J Clin Microbiol June 2000;38(6):2227-31

2: Trajman, A et al, 1997, Int. J. Tuberc. Lung Dis. 1, 498-501

3: Behr M A, Wilson M A, Gill W P, Salamon H, Schoolnik G K, Rane S, Small P M. Science May 28, 1999;284(5419):1520-3

4: Cole R A, Lu H M, Shi Y Z, Wang J, De-Hua T, Zhou A T. Tuber Lung Dis August 1996;77(4):363-8

5: Lyashchenko, K. et al 1998 Infection and Immunity 66 (8): 3936-3940

6: Bothamley, G H and Rudd, R M. Eur. Respir. J. 1994, 7(2): 240-6

It is to be understood that this invention is not limited to the particular methodology, protocols, formulations and reagents described, as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise. Thus, for example, reference to “a complex” includes a plurality of such complexes and reference to “the formulation” includes reference to one or more formulations and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

What is claimed is:

1. A composition comprising a combination of two or more substantially pure polypeptides, which comprises one or more amino acid sequences encoded by the sequence selected from:
(a) Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76)
(b) an epitope of any one of the sequences in (a); and/or
(c) an amino acid sequence analog having at least 70% sequence identity to any one of the sequences in (a) or (b) and at the same time being antigenic.

2. A composition according to claim 1 comprising one or more fusion polypeptides, which comprises one or more amino acid sequences encoded by the sequence selected from:
(a) Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76);
(b) an epitope of any one of the sequences in (a); and/or
(c) an amino acid sequence analog having at least 70% sequence identity to any one of the sequences in (a) or (b) and at the same time being antigenic.

3. A kit comprising a combination of two or more substantially pure polypeptides, which comprises one or more amino acid sequences encoded by the sequence selected from:
(a) Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76);
(b) an epitope of any one of the sequences in (a); and/or
(c) an amino acid sequence analog having at least 70% sequence identity to any one of the sequences in (a) or (b) and at the same time being antigenic.

4. A substantially pure polypeptide, which comprises an amino acid sequence selected from
(a) Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76);
(b) an epitope of any one of the sequences in (a); and/or
(c) an amino acid sequence analog having at least 70% sequence identity to any one of the sequences in (a) or (b) and at the same time being antigenic.

5. An isolated nucleic acid comprising:
(a) one or more nucleic acid sequences having a sequence set forth in Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76); or
(b) a nucleic acid of at least 10 nucleotides in length that hybridizes under stringent hybridization conditions with a sequence set forth in Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76).

6. The nucleic acid fragment according to claim 5, wherein said nucleic acid is DNA.

7. The nucleic acid fragment according to claim 5, wherein said nucleic acid is RNA.

8. A replicable expression vector, comprising a nucleic acid as set forth in claim 5.

9. A transformed cell comprising the vector set forth in claim 8.

10. A method for producing a deletion polypeptide, comprising
culturing a host cell according to claim 9 in a culture medium under conditions sufficient to effect expression of the encoded polypeptide, and recovering the polypeptide from the host cell or culture medium.

11. A method of producing a deletion polypeptide, comprising:
isolating a polypeptide from a mycobacterium, fractions thereof, or culture filtrates thereof, said mycobacterium being selected from the group consisting of Mycobacterium tuberculosis, Mycobacterium africanum and Mycobacterium bovis, from culture filtrate or from lysates or fractions thereof;
wherein the deletion polypeptide comprises (a) the amino acid sequence set forth in Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76); (b) an epitope of any one of the sequences in (a); (c) and/or an amino acid sequence analog having at least 70% sequence identity to any one of the sequences in (a) or (b) and at the same time being antigenic

12. A method of producing a deletion polypeptide, comprising:
synthesizing the deletion polypeptide by solid or liquid phase peptide synthesis;
wherein the deletion polypeptide comprises (a) the amino acid sequence set forth in Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76); (b) an epitope of any one of the sequences in (a); (c) and/or an amino acid sequence analog having at least 70% sequence identity to any one of the sequences in (a) or (b) and at the same time being antigenic.

13. An antibody or specific binding fragment thereof, which specifically binds to a deletion polypeptide;
wherein the deletion polypeptide comprises (a) the amino acid sequence set forth in Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76); (b) an epitope of any one of the sequences in (a); (c) and/or an amino acid sequence analog having at least 70% sequence identity to any one of the sequences in (a) or (b) and at the same time being antigenic.

14. A method for diagnosing previous or ongoing infection with a virulent mycobacterium in a subject, said method comprising
contacting a sample from said subject with a two or more deletion polypeptides, wherein the deletion polypeptide comprises (a) the amino acid sequence set forth in Rv1516 (SEQ ID NO: 53), Rv1575 (SEQ ID NO: 32), Rv1586 (SEQ ID NO: 21), Rv2660 (SEQ ID NO: 100), Rv3118 (SEQ ID NO: 39), Rv1976 (SEQ ID NO: 122), Rv1966 (SEQ ID NO: 112), Rv3618 (SEQ ID NO: 71) and Rv3429 (SEQ ID NO: 76); (b) an epitope of any one of the sequences in (a); (c) and/or an amino acid sequence analog having at least 70% sequence identity to any one of the sequences in (a) or (b) and at the same time being antigenic,
detecting specific immunoreactivity between said patient sample and said deletion polypeptide, wherein the presence of said immunoreactivity indicates that said subject is infected by Mycobacterium tuberculosis or has been infected by Mycobacterium tuberculosis.

15. The method according to claim 14, wherein said sample comprises antibodies produced by said subject.

16. The method according to claim 15, wherein said sample is a blood sample or a derivative thereof.

17. The method according to claim 15, wherein said two or more deletion polypeptides are combined in a single test sample.

18. The method according to claim 17, wherein said deletion polypeptides are combined in a fusion polypeptide.

19. The method according to claim 15, wherein said two or more deletion polypeptides are tested in individual test samples.

20. A method for diagnosing previous or ongoing infection with a virulent mycobacterium in a subject, said method comprising
contacting a subject sample with an antibody according to claim 13,
detecting specific binding of said antibody with said subject sample, said specific binding being an indication that said subject is infected by Mycobacterium tuberculosis or has been infected by Mycobacterium tuberculosis.

21. The method according to claim 20, wherein said subject sample is a tracheal lavage sample or a blood sample.


SEQUENCE LISTING (137 sequences)

SEQ ID NO: 1 - DNA, 1773 bp, Mycobacteria tuberculosis
atgactgctgaaccggaagtacggacgctgcgcgaggttgtgctggaccagctcggcactgctgaatcgcgtgcgtacaagatgtggctgccgccgttgaccaatccggtcccgctcaacgagctcatcgcccgtgatcggcgacaacccctgcgatttgccctggggatcatggatgaaccgcgccgccatctacaggatgtgtggggcgtagacgtttccggggccggcggcaacatcggtattgggggcgcacctcaaaccgggaagtcgacgctactgcagacgatggtgatgtcggccgccgccacacactcaccgcgcaacgttcagttctattgcatcgacctaggtggcggcgggctgatctatctcgaaaaccttccacacgtcggtggggtagccaatcggtccgagcccgacaaggtcaaccgggtggtcgcagagatgcaagccgtcatgcggcaacgggaaaccaccttcaaggaacaccgagtgggctcgatcgggatgtaccggcagctgcgtgacgatccaagtcaacccgttgcgtccgatccatacggcgacgtctttctgatcatcgacggatggcccggttttgtcggcgagttccccgaccttgaggggcaggttcaagatctggccgcccaggggctggcgttcggcgtccacgtcatcatctccacgccacgctggacagagctgaagtcgcgtgttcgcgactacctcggcaccaagatcgagttccggcttggtgacgtcaatgaaacccagatcgaccggattacccgcgagatcccggcgaatcgtccgggtcgggcagtgtcgatggaaaagcaccatctgatgatcggcgtgcccaggttcgacggcgtgcacagcgccgataacctggtggaggcgatcaccgcgggggtgacgcagatcgcttcccagcacaccgaacaggcacctccggtgcgggtcctgccggagcgtatccacctgcacgaactcgacccgaacccgccgggaccagagtccgactaccgcactcgctgggagattccgatcggcttgcgcgagacggacctgacgccggctcactgccacatgcacacgaacccgcacctactgatcttcggtgcggccaaatcgggcaagacgaccattgcccacgcgatcgcgcgcgccatttgtgcccgaaacagtccccagcaggtgcggttcatgctcgcggactaccgctcgggcctgctggacgcggtgccggacacccatctgctgggcgccggcgcgatcaaccgcaacagcgcgtcgctagacgaggccgttcaagcactggcggtcaacctgaagaagcggttgccgccgaccgacctgacgacggcgcagctacgctcgcgttcgtggtggagcggatttgacgtcgtgcttctggtcgacgattggcacatgatcgtgggtgccgccggggggatgccgccgatggcaccgctggccccgttattgccggcggcggcagatatcgggttgcacatcattgtcacctgtcagatgagccaggcttacaaggcaaccatggacaagttcgtcggcgccgcattcgggtcgggcgctccgacaatgttcctttcgggcgagaagcaggaattcccatccagtgagttcaaggtcaagcggcgcccccctggccaggcatttctcgtctcgccagacggcaaagaggtcatccaggccccctacatcgagcctccagaagaagtgttcgcagcacccccaagcgccggt

SEQ ID NO: 2 - DNA, 297 bp, Mycobacteria tuberculosis
atggaaaaaatgtcacatgatccgatcgctgccgacattggcacgcaagtgagcgacaacgctctgcacggcgtgacggccggctcgacggcgctgacgtcggtgaccgggctggttcccgcgggggccgatgaggtctccgcccaagcggcgacggcgttcacatcggagggcatccaattgctggcttccaatgcatcggcccaagaccagctccaccgtgcgggcgaagcggtccaggacgtcgcccgcacctattcgcaaatcgacgacggcgccgccggcgtcttcgccgaa

SEQ ID NO: 3 - DNA, 1104 bp, Mycobacteria tuberculosis
atgctgtggcacgcaatgccaccggagctaaataccgcacggctgatggccggcgcgggtccggctccaatgcttgcggcggccgcgggatggcagacgctttcggcggctctggacgctcaggccgtcgagttgaccgcgcgcctgaactctctgggagaagcctggactggaggtggcagcgacaaggcgcttgcggctgcaacgccgatggtggtctggctacaaaccgcgtcaacacaggccaagacccgtgcgatgcaggcgacggcgcaagccgcggcatacacccaggccatggccacgacgccgtcgctgccggagatcgccgccaaccacatcacccaggccgtccttacggccaccaacttcttcggtatcaacacgatcccgatcgcgttgaccgagatggattatttcatccgtatgtggaaccaggcagccctggcaatggaggtctaccaggccgagaccgcggttaacacgcttttcgagaagctcgagccgatggcgtcgatccttgatcccggcgcgagccagagcacgacgaacccgatcttcggaatgccctcccctggcagctcaacaccggttggccagttgccgccggcggctacccagaccctcggccaactgggtgagatgagcggcccgatgcagcagctgacccagccgctgcagcaggtgacgtcgttgttcagccaggtgggcggcaccggcggcggcaacccagccgacgaggaagccgcgcagatgggcctgctcggcaccagtccgctgtcgaaccatccgctggctggtggatcaggccccagcgcgggcgcgggcctgctgcgcgcggagtcgctacctggcgcaggtgggtcgttgacccgcacgccgctgatgtctcagctgatcgaaaagccggttgccccctcggtgatgccggcggctgctgccggatcgtcggcgacgggtggcgccgctccggtgggtgcgggagcgatgggccagggtgcgcaatccggcggctccaccaggccgggtctggtcgcgccggcaccgctcgcgcaggagcgtgaagaagacgacgaggacgactgggacgaagaggacgactgg

SEQ ID NO: 4 - DNA, 300 bp, Mycobacteria tuberculosis
atggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttc

SEQ ID NO: 5 - DNA, 285 bp, M. tuberculosis
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgca

SEQ ID NO: 6 - DNA, 1998 bp, Mycobacteria tuberculosis
atggcggccgactacgacaagctcttccggccgcacgaaggtatggaagctccggacgatatggcagcgcagccgttcttcgaccccagtgcttcgtttccgccggcgcccgcatcggcaaacctaccgaagcccaacggccagactccgcccccgacgtccgacgacctgtcggagcggttcgtgtcggccccgccgccgccacccccacccccacctccgcctccgccaactccgatgccgatcgccgcaggagagccgccctcgccggaaccggccgcatctaaaccacccacaccccccatgcccatcgccggacccgaaccggccccacccaaaccacccacaccccccatgcccatcgccggacccgaaccggccccacccaaaccacccacacctccgatgcccatcgccggacctgcacccaccccaaccgaatcccagttggcgccccccagaccaccgacaccacaaacgccaaccggagcgccgcagcaaccggaatcaccggcgccccacgtaccctcgcacgggccacatcaaccccggcgcaccgcaccagcaccgccctgggcaaagatgccaatcggcgaacccccgcccgctccgtccagaccgtctgcgtccccggccgaaccaccgacccggcctgccccccaacactcccgacgtgcgcgccggggtcaccgctatcgcacagacaccgaacgaaacgtcgggaaggtagcaactggtccatccatccaggcgcggctgcgggcagaggaagcatccggcgcgcagctcgcccccggaacggagccctcgccagcgccgttgggccaaccgagatcgtatctggctccgcccacccgccccgcgccgacagaacctccccccagcccctcgccgcagcgcaactccggtcggcgtgccgagcgacgcgtccaccccgatttagccgcccaacatgccgcggcgcaacctgattcaattacggccgcaaccactggcggtcgtcgccgcaagcgtgcagcgccggatctcgacgcgacacagaaatccttaaggccggcggccaaggggccgaaggtgaagaaggtgaagccccagaaaccgaaggccacgaagccgcccaaagtggtgtcgcagcgcggctggcgacattgggtgcatgcgttgacgcgaatcaacctgggcctgtcacccgacgagaagtacgagctggacctgcacgctcgagtccgccgcaatccccgcgggtcgtatcagatcgccgtcgtcggtctcaaaggtggggctggcaaaaccacgctgacagcagcgttggggtcgacgttggctcaggtgcgggccgaccggatcctggctctagacgcggatccaggcgccggaaacctcgccgatcgggtagggcgacaatcgggcgcgaccatcgctgatgtgcttgcagaaaaagagctgtcgcactacaacgacatccgcgcacacactagcgtcaatgcggtcaatctggaagtgctgccggcaccggaatacagctcggcgcagcgcgcgctcagcgacgccgactggcatttcatcgccgatcctgcgtcgaggttttacaacctcgtcttggctgattgtggggccggcttcttcgacccgctgacccgcggcgtgctgtccacggtgtccggtgtcgtggtcgtggcaagtgtctcaatcgacggcgcacaacaggcgtcggtcgcgttggactggttgcgcaacaacggttaccaagatttggcgagccgcgcatgcgtggtcatcaatcacatcatgccgggagaacccaatgtcgcagttaaagacctggtgcggcatttcgaacagcaagttcaacccggccgggtcgtggtcatgccgtgggacaggcacattgcggccggaaccgagatttcactcgacttgctcgaccctatctacaagcgcaaggtcctcgaattggccgcagcgctatccgacgatttcgagagggctggacgtcgt

SEQ ID NO: 7 - DNA, 1533 bp, Mycobacteria tuberculosis
ttgagcgcacctgctgttgctgctggtcctaccgccgcgggggcaaccgctgcgcggcctgccaccacccgggtgacgatcctgaccggcagacggatgaccgatttggtactgccagcggcggtgccgatggaaacttatattgacgacaccgtcgcggtgctttccgaggtgttggaagacacgccggctgatgtactcggcggcttcgactttaccgcgcaaggcgtgtgggcgttcgctcgtcccggatcgccgccgctgaagctcgaccagtcactcgatgacgccggggtggtcgacgggtcactgctgactctggtgtcagtcagtcgcaccgagcgctaccgaccgttggtcgaggatgtcatcgacgcgatcgccgtgcttgacgagtcacctgagttcgaccgcacggcattgaatcgctttgtgggggcggcgatcccgcttttgaccgcgcccgtcatcgggatggcgatgcgggcgtggtgggaaactgggcgtagcttgtggtggccgttggcgattggcatcctggggatcgctgtgctggtaggcagcttcgtcgcgaacaggttctaccagagcggccacctggccgagtgcctactggtcacgacgtatctgctgatcgcaaccgccgcagcgctggccgtgccgttgccgcgcggggtcaactcgttgggggcgccacaagttgccggcgccgctacggccgtgctgtttttgaccttgatgacgcggggcggccctcggaagcgtcatgagttggcgtcgtttgccgtgatcaccgctatcgcggtcatcgcggccgccgctgccttcggctatggataccaggactgggtccccgcgggggggatcgcattcgggctgttcattgtgacgaatgcggccaagctgaccgtcgcggtcgcgcggatcgcgctgccgccgattccggtacccggcgaaaccgtggacaacgaggagttgctcgatcccgtcgcgaccccggaggctaccagcgaagaaaccccgacctggcaggccatcatcgcgtcggtgcccgcgtccgcggtccggctcaccgagcgcagcaaactggccaagcaacttctgatcggatacgtcacgtcgggcaccctgattctggctgccggtgccatcgcggtcgtggtgcgcgggcacttctttgtacacagcctggtggtcgcgggtttgatcacgaccgtctgcggatttcgctcgcggctttacgccgagcgctggtgtgcgtgggcgttgctggcggcgacggtcgcgattccgacgggtctgacggccaaactcatcatctggtacccgcactatgcctggctgttgttgagcgtctacctcacggtagccctggttgcgctcgtggtggtcgggtcgatggctcacgtccggcgcgtttcaccggtcgtaaaacgaactctggaattgatcgacggcgccatgatcgctgccatcattcccatgctgctgtggatcaccggggtgtacgacacggtccgcaatatccggttc

SEQ ID NO: 8 - DNA, 840 bp, Mycobacteria tuberculosis
atggctgaaccgttggccgtcgatcccaccggcttgagcgcagcggccgcgaaattggccggcctcgtttttccgcagcctccggcgccgatcgcggtcagcggaacggattcggtggtagcagcaatcaacgagaccatgccaagcatcgaatcgctggtcagtgacgggctgcccggcgtgaaagccgccctgactcgaacagcatccaacatgaacgcggcggcggacgtctatgcgaagaccgatcagtcactgggaaccagtttgagccagtatgcattcggctcgtcgggcgaaggcctggctggcgtcgcctcggtcggtggtcagccaagtcaggctacccagctgctgagcacacccgtgtcacaggtcacgacccagctcggcgagacggccgctgagctggcaccccgtgttgttgcgacggtgccgcaactcgttcagctggctccgcacgccgttcagatgtcgcaaaacgcatcccccatcgctcagacgatcagtcaaaccgcccaacaggccgcccagagcgcgcagggcggcagcggcccaatgcccgcacagcttgccagcgctgaaaaaccggccaccgagcaagcggagccggtccacgaagtgacaaacgacgatcagggcgaccagggcgacgtgcagccggccgaggtcgttgccgcggcacgtgacgaaggcgccggcgcatcaccgggccagcagcccggcgggggcgttcccgcgcaagccatggataccggagccggtgcccgcccagcggcgagtccgctggcggcccccgtcgatccgtcgactccggcaccctcaacaaccacaacgttg

SEQ ID NO: 9 - DNA, 2187 bp, Mycobacteria tuberculosis
atgagtattaccaggccgacgggcagctatgccagacagatgctggatccgggcggctgggtggaagccgatgaagacactttctatgaccgggcccaggaatatagccaggttttgcaaagggtcaccgatgtattggacacctgccgccagcagaaaggccacgtcttcgaaggcggcctatggtccggcggcgccgccaatgctgccaacggcgccctgggtgcaaacatcaatcaattgatgacgctgcaggattatctcgccacggtgattacctggcacaggcatattgccgggttgattgagcaagctaaatccgatatcggcaataatgtggatggcgctcaacgggagatcgatatcctggagaatgaccctagcctggatgctgatgagcgccataccgccatcaattcattggtcacggcgacgcatggggccaatgtcagtctggtcgccgagaccgctgagcgggtgctggaatccaagaattggaaacctccgaagaacgcactcgaggatttgcttcagcagaagtcgccgccacccccagacgtgcctaccctggtcgtgccatccccgggcacaccgggcacaccgggaaccccgatcaccccgggaaccccgatcaccccgggaaccccaatcacacccatcccgggagcgccggtaactccgatcacaccaacgcccggcactcccgtcacgccggtgaccccgggcaagccggtcaccccggtgaccccggtcaaaccgggcacaccaggcgagccaaccccgatcacgccggtcacccccccggtcgccccggccacaccggcaaccccggccacgcccgttaccccagctcccgctccacacccgcagccggctccggcaccggcgccatcgcctgggccccagccggttacaccggccactcccggtccgtctggtccagcaacaccgggcaccccagggggcgagccggcgccgcacgtcaaacccgcggcgttggcggagcaacctggtgtgccgggccagcatgcgggcggggggacgcagtcggggcctgcccatgcggacgaatccgccgcgtcggtgacgccggctgcggcgtccggtgtcccgggcgcacgggcggcggccgccgcgccgagcggtaccgccgtgggagcgggcgcgcgttcgagcgtgggtacggccgcggcctcgggcgcggggtcgcatgctgccactgggcgggcgccggtggctacctcggacaaggcggcggcaccgagcacgcgggcggcctcggcgcggacggcacctcctgcccgcccgccgtcgaccgatcacatcgacaaacccgatcgcagcgagtctgcagatgacggtacgccggtgtcgatgatcccggtgtcggcggctcgggcggcacgcgacgccgccactgcagctgccagcgcccgccagcgtggccgcggtgatgcgctgcggttggcgcgacgcatcgcggcggcgctcaacgcgtccgacaacaacgcgggcgactacgggttcttctggatcaccgcggtgaccaccgacggttccatcgtcgtggccaacagctatgggctggcctacatacccgacgggatggaattgccgaataaggtgtacttggccagcgcggatcacgcaatcccggttgacgaaattgcacgctgtgccacctacccggttttggccgtgcaagcctgggcggctttccacgacatgacgctgcgggcggtgatcggtaccgcggagcagttggccagttcggatcccggtgtggccaagattgtgctggagccagatgacattccggagagcggcaaaatgacgggccggtcgcggctggaggtcgtcgacccctcggcggcggctcagctggccgacactaccgatcagcgtttgctcgacttgttgccgccggcgccggtggatgtcaatccaccgggcgatgagcggcacatgctgtggttcgagctgatgaagcccatgaccagcaccgctaccggccgcgaggccgctcatctgcgggcgttccgggcctacgctgcccactcacaggagattgccctgcaccaagcgcacactgcgactgacgcggccgtccagcgtgtggccgtcgcggactggctgtactggcaatacgtcaccgggttgctcgaccgggccctggccgccgcatgc

SEQ ID NO: 10 - DNA, 426 bp, Mycobacteria tuberculosis
atggccggactgaacatttacgtgaggcgctggcggacagcgcttcacgcaaccgtgtcggcattgatagttgccatcctcggactcgccatcaccccggtcgctagtgcggcgacggccagggcgacgttgtcggtgacatcgacgtggcagaccggtttcatcgcccgcttcaccatcacaaactcgagcacggcgccgctaaccgattggaagcttgaattcgacttgccggcaggagaatccgtcttgcacacatggaatagcaccgttgcacgatctggcacgcactacgttctcagcccagcgaattggaatcgcatcattgcccccggtggttcagccacgggcggcctaagaggcgggctgaccggttcttactcgccgccgtcgagttgtctgctcaacgggcaatatccttgcacc

SEQ ID NO: 11 - DNA, 597 bp, Mycobacteria tuberculosis
gtgaactcaccactggtcgtcggcttcctggcctgcttcacgctgatcgccgcgattggcgcgcagaacgcattcgtgctgcggcagggaatccagcgtgagcacgtgctgccggtggtggcgctgtgcacggtgtccgacatcgtgctgatcgccgccggtatcgcggggttcggcgcattgatcggcgcacatccgcgtgcgctcaatgtcgtcaagtttggcggcgccgccttcctaatcggctacgggctacttgcggcccggcgggcgtggcgacctgttgcgctgatcccatctggcgccacgccggttcgcttagccgaggtcctggtgacctgtgcggcattcacgttcctcaacccacacgtctacctcgacaccgtcgtgttgctaggcgcgctggccaacgagcacagcgaccagcgctggctgttcggcctcggcgcggtcacagccagtgcggtatggttcgccaccctcgggttcggagccggccggttgcgcgggctgttcaccaaccccggctcgtggagaatcctcgacggcctgatcgcggtcatgatggttgcgctgggaatctcgctgaccgtgacc

SEQ ID NO: 12 - DNA, 909 bp, Mycobacteria tuberculosis
atggtggatccgcagcttgacggtccacagctggccgcattggctgccgtggtcgaactgggcagcttcgatgcggccgcggagcgcctacatgtcaccccgtcggctgtcagtcagcgcatcaagtcgttggagcagcaggtcggccaggtgctggtggtcagggaaaagccatgtcgggcgacgaccgcaggtatcccgctgttgcggttggccgcgcaaacagcgttgctcgagtccgaggcgctcgctgaaatgggtggcaacgcgtcgctgaaacgcacgcggatcaccattgcggtaaacgccgattccatggcgacatggttttcggccgtgttcgacggtctcggcgacgtcctgctcgacgttcggatcgaggaccaggaccattccgcgcggctgctacgggagggtgtggcgatgggcgcggtgaccaccgagcggaacccggtgccgggctgccgggtgcacccgctgggtgaaatgcgctacctaccagtggccagcaggccattcgtccagcgccatctatccgacgggttcactgccgccgcggcggctaaagctccgtcactggcgtggaatcgtgacgatgggctgcaggacatgttggtgcgtaaggcctttcgtcgcgccatcaccagaccgacgcactttgtcccgaccacagagggcttcaccgccgcagcgcgcgccgggctgggatggggcatgttccccgagaagctggcagcatctccgcttgccgatggatcgttcgtacgggtctgcgacatacacctcgacgtccctctctattggcaatgctggaaactggacagtccgatcatcgcgcgaattaccgacacggtgagggcggcggcaagcggtctgtaccggggccagcaacgccgccgccgaccgggt

SEQ ID NO: 13 - DNA, 651 bp, Mycobacteria tuberculosis
atgactccacgcagccttgttcgcatcgttggtgtcgtggttgcgacgaccttggcgctggtgagcgcacccgccggcggtcgtgccgcgcatgcggatccgtgttcggacatcgcggtcgttttcgctcgcggcacgcatcaggcttctggtcttggcgacgtcggtgaggcgttcgtcgactcgcttacctcgcaagttggcgggcggtcgattggggtctacgcggtgaactacccagcaagcgacgactaccgcgcgagcgcgtcaaacggttccgatgatgcgagcgcccacatccagcgcaccgtcgccagctgcccgaacaccaggattgtgcttggtggctattcgcagggtgcgacggtcatcgatttgtccacctcggcgatgccgcccgcggtggcagatcatgtcgccgctgtcgcccttttcggcgagccatccagtggtttctccagcatgttgtggggcggcgggtcgttgccgacaatcggtccgctgtatagctctaagaccataaacttgtgtgctcccgacgatccaatatgcaccggaggcggcaatattatggcgcatgtttcgtatgttcagtcggggatgacaagccaggcggcgacattcgcggcgaacaggctcgatcacgccgga

SEQ ID NO: 14 - DNA, 1674 bp, Mycobacteria tuberculosis
gtgtcatttctggtcgtggttcccgagttcttgacgtccgcggcagcggatgtggagaacataggttccacactgcgcgcggcgaatgccgcggctgccgcctcgaccaccgcgcttgcggccgctggcgctgatgaggtatcggcggcggtggcagcgctgtttgccaggttcggtcaggaatatcaagcggtcagcgcgcaggcgagcgctttccatcaacagttcgtgcagacgctgaactcggcgtcaggatcgtatgcggccgcggaggccaccatcgcgtcacagttgcagaccgcgcagcacgatctgctgggcgcggtcaatgcaccaaccgaaacgttgttggggcgtccgctaatcggcgacggagcacccgggacggcaacgagtccgaatggcggggcgggtgggctgctgtacggcaacggcggcaacggttattccgcgacggcgtcgggggtcggcggcggggccggcggttccgcggggttgatcggcaatggcggcgccgggggagccggcggacccaacgcccccgggggagccggcggcaacggtggctggctgctcggcaacggcgggatcggcgggcccgggggcgcgtcgagcatccccggcatgagtggtggagccggcggaaccggcggtgccgcaggacttttgggctggggagcgaacggcggagccggcggcctcggtgatggagtcggtgtcgatcgtggcacgggcggcgccggaggccgcggcggcctgttgtatggcggatacggcgtcagtgggccaggcggcgacggcagaaccgtcccgctggagataattcatgtcacagagccgacggtacatgccaacgtcaacggcggaccgacgtcaaccattctggtcgacaccggatccgctggtcttgttgtctcgcctgaggatgtcgggggaatcctgggagtgcttcacatgggcctcccaaccggattgagcatcagcggttacagcggggggctgtactacatcttcgccacgtataccacgacggtggacttcgggaatggcatcgtcaccgcgccgaccgccgttaatgtcgtcctcttgtccatcccaacgtcccccttcgccatttcgacctacttcagcgccttgctggccgatccgacaacaactccgttcgaagcctatttcggtgccgtcggcgtggacggcgttctgggagttgggcccaatgcggtgggaccaggccccagcattccgacgatggcgttaccgggtgacctcaaccagggagtgctcatcgacgcacccgcaggtgagctcgtgttcggtcccaacccgctacctgcgcccaacgtcgaggtcgtcggatcgccgatcaccaccctgtacgtaaagatcgatggtgggactcccatacccgtcccctcgatcatcgattccggtggggtaacgggaaccatcccgtcatatgtcatcggatccggaaccctgccggcgaacacaaacattgaggtctacaccagccccggcggtgatcggctctacgcgttcaacacaaacgattaccgcccgaccgtcatttcatccggcctgatgaataccgggttcttgcccttcagattccagccggtgtacatcgactacagccccagcggtatagggacaacagtctttgatcatccggcg

SEQ ID NO: 15 - DNA, 1674 bp, M. tuberculosis; identical to SEQ ID NO: 14

SEQ ID NO: 16 - DNA, 417 bp, Mycobacteria tuberculosis
atgatcgtggacacaagcgccgtggtggccctggttcaaggcgagcggccgcacgccaccctggtcgcggccgccctggccggcgcccatagccccgtcatgtctgcacccaccgtcgccgaatgcctgattgtcttgaccgcccgtcacggccccgttgcgcgcacgatcttcgaacgacttcgcagcgaaatcggcttgagcgtgtcatctttcaccgccgagcatgccgctgccacgcaacgagcctttctgcgatacggcaaggggcgccaccgcgcggctctcaacttcggagactgtatgacgtacgcgaccgcccagctgggccaccaaccactgctggccgtcggcaacgacttcccgcaaaccgaccttgagttccgcggcgtcgtcggctactggccaggcgtcgcg

SEQ ID NO: 17 - DNA, 684 bp, M. tuberculosis
gtgcgcatcaagatcttcatgctggtcacggctgtcgttttgctctgttgttcgggtgtggccacggccgcgcccaagacctactgcgaggagttgaaaggcaccgataccggccaggcgtgccagattcaaatgtccgacccggcctacaacatcaacatcagcctgcccagttactaccccgaccagaagtcgctggaaaattacatcgcccagacgcgcgacaagttcctcagcgcggccacatcgtccactccacgcgaagccccctacgaattgaatatcacctcggccacataccagtccgcgataccgccgcgtggtacgcaggccgtggtgctcaaggtctaccagaacgccggcggcacgcacccaacgaccacgtacaaggccttcgattgggaccaggcctatcgcaagccaatcacctatgacacgctgtggcaggctgacaccgatccgctgccagtcgtcttccccattgtgcaaggtgaactgagcaagcagaccggacaacaggtatcgatagcgccgaatgccggcttggacccggtgaattatcagaacttcgcagtcacgaacgacggggtgattttcttcttcaacccgggggagttgctgcccgaagcagccggcccaacccaggtattggtcccacgttccgcgatcgactcgatgctggcc

SEQ ID NO: 18 - DNA, 684 bp, M. tuberculosis; identical to SEQ ID NO: 17

SEQ ID NO: 19 - DNA, 1443 bp, Mycobacteria tuberculosis
gtggtcggcccgcggacgagaggatatgcgatccacaagctgggtttctgcagcgtcgtcatgctcgggatcaactcgataatcggcgccggtatcttcctaactccaggtgaggtgatcgggctcgcaggacccttcgcgccgatggcctatgttttagctggcattttcgcgggtgtcgtggcgatcgtcttcgcgacggcggcaaggtacgtcagaacaaacggtgcctcctacgcctacacaacggccgcatttgggcgccggatcggcatctatgtcggtgtcacccacgccattaccgcgtccatcgcttggggggtgttggcttcttttttcgtctcgacgctgttgcgagtggccttccccgacaaggcctgggccgacgccgagcaactgttcagtgtgaagacgctgacgtttctcggctttatcggcgtgctgttggccatcaacctcttcggcaaccgggcgatcaagtgggccaacggaacgtcaacggtaggcaaggcattcgcgctctcggcattcattgtcggcgggctgtggatcatcaccacccagcacgtgaacaactacgcaacggcgtggtcggcatacagcgcgaccccgtactcgttgcttggcgtcgccgaaattggcaagggcacgttctcgagtatggcgctggccacgattgtcgcgttgtacgcattcaccggtttcgaatcgatcgcgaacgccgccgaagaaatggacgcgccggaccggaacctgccgagagctataccgatcgcgatcttctcggttggcgcgatctacttgctcaccctaacggtagcgatgctgctcggatcgaacaagatcgccgcgtcggacgacaccgtgaaactggccgcggccatcggaaacgctaccttccgaacgatcatcgtcgtcggagccctgatatcgatgttcggcatcaatgtcgcggcctcgttcggtgcaccgcggctttggaccgcgttagcggacagcggggttctgccgacacgcttgtcacgcaagaaccaatacgacgtgccgatggtctccttcgcaattacggcgtcgttggcgctcgcattcccgttggcgctgcggttcgacaacctgcacctgaccggcctggcggtgatcgcccgattcgtccagttcatcatcgtgccgatcgctctcatcgcattggcgaggtctcaggcagtagaacatgctgctgtgcggcgaaatgcgttcaccgacaaggtgttaccgcttgttgcgatcgtggtctcggttgggctggcagtgtcctacgactaccgctgcatctttctagtgcggggtggtccgaactacttctcgattgctttgatcgtgatcacgttcgtcgtggtaccggcgatggcttatctgcactactaccgaatcattcgccgggttggcgatcggccgagcactcgc

SEQ ID NO: 20 - DNA, 846 bp, Mycobacteria tuberculosis
atgggtgaggcgaacatccgcgagcaggcgatcgccacgatgccacggggtggccccgacgcgtcttggctggatcgtcgattccagaccgacgcactggagtacctcgaccgcgacgatgtgcccgatgaggtcaaacagaagatcatcggggtgctcgaccgggtgggcaccctgaccaacctgcacgagaagtacgcccggatagccctgaaacttgtttctgacattcccaacccgcgaatcctggaacttggtgcgggccatggcaagctctcagcgaaaatcctcgagctacacccgacagcgacggtgacgatcagcgatctagatcccacctcggtggccaacatcgccgcgggagagctgggaacacatccgcgagcacgcacccaagtgatcgacgccaccgcaatcgacggccacgaccacagctatgacctggcggtcttcgcgctggcatttcaccacctgccgcctacggtcgcctgcaaagcgatcgccgaggccacccgggtggggaagcgctttctgatcatcgacctcaaacggcagaaaccgctgtcgttcacgctctcttcggtgctgctactgccgctccacctactgctgctgccatggtcgtcgatgcgctcgagcatgcacgacggctttatcagcgcactacgtgcctacagtccctcggcgttgcagacgcttgcccgcgccgccgatccgggaatgcaggttgaaatcttgcccgcaccgaccaggctattcccgccatcgctcgccgttgtgttctcccgttcgagctcagcgccaacggaatctagcgagtgctcggccgatcgccaacccggcgaa

SEQ ID NO: 21 - DNA, 1407 bp, Mycobacteria tuberculosis
gtgagatacactacacctgtgcgtgctgctgtctacctccgaatctcagaagaccgctccggcgaacagctcggcgtggcccgccaacgcgaggactgcctaaagctgtgcgggcagcgaaaatgggtgcccgtcgagtacctcgacaacgacgtcagcgcatcaaccggcaagcgccgccccgcctacgagcagatgttggccgacatcaccgccggcaagatcgccgccgtggtggcctgggacctggaccggctccatcgccgtcccatcgagctggaagccttcatgtcattagccgacgagaagcggctggccctggccaccgtcgccggcgacgttgacctggcgacaccccagggccggctagtcgcccgcctgaaggggtcggtggccgctcacgaaaccgagcacaagaaggcacgacagcgccgcgccgcccgccagaaagctgaacgcggccaccccaactggtcgaaagccttcggctacctgcccggccccaacggtcccgaacccgacccccggacagcgccgctggtcaaacaggcctacgccgacatcctcgccggggcgtccctgggcgacgtgtgccgccagtggaacgacgccggggcgttcaccatcaccggccgcccgtggacgactacaacgctgtcgaaattcttgcgcaaaccccgcaacgccggactacgcgcatataagggtgcccgctacggcccggtggaccgcgacgcgattgtcggcaaggcccagtggtcgccgctggtggacgaggcgacgttctgggccgcccaggccgtgctggacgcccccggccgcgcccccggccgcaaaagcgtgcgccgccacctgctgaccgggctggcaggctgcggcaaatgcggcaaccacctggccggcagctaccgcaccgacggccaggtcgtctacgtgtgcaaggcgtgccacggggtggccatcctggccgacaacatcgaaccgatcctgtatcacatcgtggccgagcggctggccatgcccgacgccgttgacttgttgcgccgggagattcacgacgccgccgaagccgaaaccatccgcctggaactggaaaccctctacggggagctggacaggctcgccgtcgaacgcgccgaagggctactgaccgcgcgccaggtgaagatcagcaccgacatcgtcaacgccaagataacgaaacttcaggcccgccaacaggatcaggaacggctccgagtgttcgacgggataccgttgggaacaccgcaagtcgccgggatgatagccgagctgtcgccggaccggttccgcgccgtcctcgacgtcctcgctgaagtcgttgtccagccggtcggcaagagcggcaggatattcaatcccgaacgggtgcaggtgaattggcga

SEQ ID NO: 22 - DNA, 513 bp, M. tuberculosis
atgagccggcaccacaacatcgtgatcgtctgtgaccacggccgcaaaggcgatggccgcatcgaacacgagcgctgcgatcttgtcgcgccgatcatttgggtcgacgagacccagggctggttaccgcaggcgccagcggtggcaacattactcgacgacgacaaccagccgcgagccgttattggcttgccgcccaacgagtctcgcctacgacctgaaatgcgccgcgacgggtgggtgcggctgcactgggaattcgcctgcctgaggtacggcgccgccggcgtgcgcacgtgcgagcagcggcccgtgcgggttcgcaacggcgacctgcaaacactgtgcgagaacgttccgcggctactgaccggactggccggcaaccccgactacgcaccgggttttgcggtgcagtcggacgcggtggtcgtcgccatgtggctgtggcgcacgctctgcgaaagcgacacgccgaacaaactacgcgccaccccaacgcgtggtagctgc

SEQ ID NO: 23 - DNA, 219 bp, M. tuberculosis
gtgtcgaccatctaccatcatcgcggccgcgtagccgcactgtctcgttcccgcgcatccgacgatcccgagttcatcgccgcgaaaaccgatctcgttgccgcgaacatcgcggactacctcatccgcaccctcgccgcagcgccgcccctgactgacgagcagcgcacccggctggccgagctgctgcgccccgtgcggcggtcaggcggtgcccga

SEQ ID NO: 24 - DNA, 396 bp, Mycobacteria tuberculosis
atgaccgccggcgccggcgggtcgccgccgacgcgacgatgcccggccacggaggaccgggcacccgcgacagtcgccacaccgtctagcgccgatcctaccgcgtcacgcgccgtgtcgtggtggtcggtgcacgagcatgtcgcgccggtcctggatgctgccgggtcgtggccgatggccggcacaccggcctggcgtcagctcgacgacgccgatcctcgcaaatgggccgcgatctgcgacgcagcccggcactgggctctgagggtagagacgtgccaggaggcgatggcgcaggcgtcacgtgacgtatctgcggccgccgactggcccggcatcgcccgcgagatcgtccgacggcgcggcgtgtacatcccgcgggcgggggtggcg

SEQ ID NO: 25 - DNA, 1413 bp, Mycobacteria tuberculosis
atggccgacatcccctacggcaccgactatcccgacgccccctggatcgaccgggacgggcacgtgctcatcgacgacggtggcaaaccgacgcaagttcatcgcggccaagcccgaatcgcctaccggctagccgaacgttaccaggacaagctgctgcacgtggccgggatcggctggcactcctgggacggcagacgctgggcagccgacgaccgcggcgaagccaaacgtgcagtgctggcagagctgcgccaagcgctctcagacagcctcaacgacaaggaattacgcgccgacgtccgaaaatgcgaatcggcgtccggcgtggccggcgtgctcgacctggccgccgcactggtaccattcgccgcgacggtagccgacctcgacagcgacccgcacttgctcaacgtcgcgaatgggacgctggacctgcacacgctcaaattgcggccccacgcgcccgctgaccgcatcacaaagatatgccgcggtgcctaccagtccgacaccgaatcgcctctctggcaagcgttcttgacccgcgttctgcccgatgaaggtgtgcgcgggttcgtgcaacgcctggccggcgtcggcctactaggcaccgtccgcgaacatgtcctggcgattcttatcggtgtaggtgccaacggaaaatctgtgttcgacaaggcgattcgctatgcccttggcgattatgcctgcaccgctgagcctgaccttttcatgcaccgggaaaacgctcacccaacaggcgaaatggacctccgcggcgtgcgatgggtagcggtatccgagagcgaaaaagatcgccggctggccgaatcaacgataaaacggctgactggcggcgacaccatccgcgcccgaaagatgcggcaagacttcgtggaattcacgccgtcacataccccactgctcatcaccaaccacctaccgagagtgcccggcgatgatacggccatctggcggcgaattcgagtggtgccgtttgaagtagtgattcctgccgacgagcaggaccgggaactggacgcacggttgcagttggaggccgacagcatcctgtcctgggcggtggccggatggagcgactatcagcgaatcggactatcccagccggacgcggtgctcgcggcaacgtcgaattaccgcgaggactccgacacgataaagaggttcatcgacgacgaatgcgtcaccagctcgccggtgctgaaagccactactacgcatctgttcgaggcgtggcaaaggtggcgggtgcaagaaggcgtacccgaaatctcgcgcaaagcgttcggccagtcgctcgacacccacggatacccggtcactgacaaggcccgtgatggtcgttggcgggccggaatagcggtgagaggggccgatgatttcgatgat

SEQ ID NO: 26 - DNA, 393 bp, Mycobacteria tuberculosis
atgaccgctgtcgcgatcaccccggcatccggcggtcggcacagcgtccgattcgcctacgactctgcgatcgtgtcgttgatcaagtccacgatccccgcctatgcccgctcctggtccgcgcacacccgctgctggttcatcgacgctgactggaccccactgctggccgccgagctgcgctaccacggccacaccgtcaccggacccgccgacccggcgcaacagcagtgcaccgactgggccaaagcgttgttccgggcggtcggaccccagcggacacccgccgtgtacagggctttatccaaagtgctgcaccccgacgccccaaccggatgcccgatactgcaacagcagctcaatgccgccagaaccgcacttaccaaccctgct

SEQ ID NO: 27 - DNA, 270 bp, Mycobacteria tuberculosis
atggctgaaacccccgaccacgccgaactgcggcgacgaatcgccgacatggctttcaacgccgatgtcggtatggcgacctgcaaacgctgtggtgacgccgtgccgtacatcatcctgccgaacctgcagaccggcgaacccgtcatgggtgtcgccgacaacaaatggaagcgcgcgaactgtcccgtcgacgtcggtaagccgtgcccgttcctaatcgccgagggtgtcgccgacagcaccgacgacaccatagaggtcgaccag

SEQ ID NO: 28 - DNA, 312 bp, Mycobacteria tuberculosis
gtgaccccgatcaaccggcccctgaccaacgacgaacgacaactgatgcacgagctggcagtccaggttgtctgctcgcagacgggttgctcacccgatgcggcggtcgaagcactcgaatccttcgcgaaagacggaacacttatcctccgcggcgacaccgagaacgcctacctcgaagccggaggcaatgttcttgtccatgccgatcgtgactggcttgccttccacgcgtcgtatcccggcaacgacccgctgcgagacgcccgacctatcgagcaggacgacgaccagggggcggggtcgccatcg

SEQ ID NO: 29 - DNA, 468 bp, Mycobacteria tuberculosis
atgccaagaccaccgaaaccggcccggctcaaactggttgagggccgctcccccggccgcgattccggcggccggaaagtccccgagtcgccgaagtttatccgtcaggcaccggatgccccggactggctcgacgccgaggcgctggccgaatggcggcgcgtcgcaccgactttggagcggcttgacctgctcaaacctgaggatcgggcgctcctgtccgcgtactgcgagacctggtccgtctacgtcgcggcggttcagcgggtccgcgccgaaggcctcacaattacctcaccgaaatccggtgtcgtgcaccggaacccggcggtgacggttgcggagacggcgcgcatgcatctgctgcgcttggcctccgagtttggcctgaccccggccgccgagcagcgactggcggtggcgccgggcgacgacggcgacgggctcaacccgtttgccccggaccgg

SEQ ID NO: 30 - DNA, 510 bp, Mycobacteria tuberculosis
atggccgagctgcggtctggcgaaggccgaaccgtgcacggcaccatcgtgccctacaacgaggcgaccaccgtccgcgacttcgacggcgagttccaggaaatgttcgctcctggcgcttttcggcgctccatcgccgagcgcggccacaaattgaagctgctggtctctcacgacgctcgaacccgctacccggtgggccgggccgttgagttgcgggaggagcctcacggcttgttcggggcgttcgagattgcggacaccccggacggcgacgaggctttggcgaacgtaaaagctggtgtcgtcgactcgttttcggtgggtttccgaccgatccgggaccgtcgcgaaggggatgtgctggtgcgcgtcgaagcggcgctgttagaggtttccctaaccggcgttccggcctattcgggggcacaaatcgccggggtgcgcgcggaatcgcttacagtcgtttcccgttcgacagccgaagcctggctgtccctactcgattgg

SEQ ID NO: 31 - DNA, 1419 bp, Mycobacteria tuberculosis
atgaccgaattcgacgacatcaaaaacctctctttacctgaaacccgtgacgcggcgaagcagctcctcgacagtgtcgccggcgacctgaccggtgaggcggcgcagcgttttcaggcgctgacgcgccacgccgaggaactgcgggcggagcagcgccgccgcggccgcgaagccgaggaggcgctgcgccgctaccgggccggtgagctgagggtggtgcccggcgctcccaccggcggcgacgacggcgacgcgccgccgggcaactcgttgcgggacaccgcgtttcgcacactggattcttgtgtgcgagacggcctgatgtcgtcgcgggcggcggagaccgcggaaaccttgtgccgcaccgggccgccgcagtccacctcgtgggcgcagcgctggctggcggccaccggcagccgcgactatttgggcgcgttcgtcaagcgggtttccaatcctgttgcggggcacacggtttggaccgaccgggaagcggccgcgtggcgtgaggctgccgcggtggccgccgagcagcgagcgatgggcctggtggacacccaaggcgggtttctgatcccggcggcgctggacccggcgatcctgctgtcgggtgatgggtcgacgaacccgattcggcaggtggcgagggtggtgcaaacgacctccgagatttggcggggcgtgacttccgaaggcgccgaagctcgttggtactccgaagcccaggaggtgtccgacgattcgccagcgttggcccagccggcggtgccgaactaccgtggaagctgctggattccgttctccatcgagctggagggtgacgcggcgagcttcgttggcgagatcggcaagattctcgcggacagcgttgagcaactgcaggccgcggcgttcgtcaacggctccggcaacggcgagcccaccgggttcgtcagcgcgctaaccggcacctccgatcaggtggtcgtcggcgcggggtcagaagcgattgtggcggcggatgtttacgcgttgcagtcggcgctgccgccaaggttccaggccagcgccgcgttcgcggcgaacttgtccaccatcaacacgttgcggcaggcggaaacttcgaatggcgcgctgaaattcccatcgctgcacgacagtccgccgatgctagccgggaagtctgtcctggaagtctcccacatggacaccgttgattcggcggtgacagcgacgaatcatccactggtgcttggcgactggaagcaattcctcatcggcgacagagttgggtccatggtggagttggtgcctcacctgttcgggccgaatcgccggccgaccgggcagcgcggattcttcgcctggttcagggtcggatcagatgtgctggtgcgcaacgcgtttcgagttctgaaggtggagactaccgcg

SEQ ID NO: 32 - DNA, 351 bp, Mycobacteria tuberculosis
atggcgccgctggccgccggatcgccgagctggaacggccgaaagccaagcagcggcaacaggaaggcggcgaccatggccgccaggctcgatattctggcttggggcccatgggccccaagccagaatcggagcgtcgttcgacgaaaacagacactgctatcggcgcagccctcggcatctccgccggcacctaccggcggctcaaacgaatcgacaacgcaacccgcagcgagttggcgcgtgggcggcccggcacccctaagcagaggccgcccacgcctggccctatcctacctacgcggtagtctccaccttcagaactcgaaacgcgttgcgcaccagcacatc

SEQ ID NO: 33 - DNA, 309 bp, Mycobacteria tuburculosis
atgggctacaaaccagaatcagagcgtcattcgacgaaaacagacactgctatcggcgcagccctcggcatctccgccggcacctaccggcggctcaaacgaatcgacaacgcaacccacagcgacgacaaagaaatccgccggttcgcggagaaacaaatggcgccgctggtcgccggatcgccgagctggaacgcccgaaagccaaggagcgccaacgcgagggtggtcgcctcggtgcatcgatcaccaatgccggctttggtcccatggaaccaaagccgtctcagcgccacactgacaaggagg

SEQ ID NO: 34 - DNA, 408 bp, Mycobacteria tuburculosis
atgaccaccacaccagcacgtttcaaccacttggtgacggtaaccgacctggaaacgggtgaccgcgccgtctgcgaccgcgaccaggtggccgagacgatccgggcgtggttcccggacgcgcccttggaggtgagggaagcgctcgttcggctgcaggccgcgttgaatcggcacgagcacaccggcgagctcgaagcgttcctgcggatcagcgtcgagcacgccgacgccgccggcggcgacgagtgcggcccggcgatcctggccggccgctccgggccggaacaagccgccatcaaccggcaactcggactcgccggcgacgacgagcccgacggcgacgacaccccgccgtggagccggatgatcgggcttggcggcggaagcccagcggaagacgagcgc

SEQ ID NO: 35 - DNA, 1407 bp, Mycobacteria tuburculosis
gtgaaacggctcagcggctgggacgcggtactgctttacagcgagaccccgaatgtgcacatgcacacactcaaggtcgccgtgatcgaattggattcggacagacaggaattcggtgtcgacgcgtttcgcgaggtgatcgctggccggctgcataagcttgagccattgggctatcagctggttgatgtcccgttgaagttccatcacccgatgtggcgggagcactgccaggtcgatctcaactaccacatccggccgtggcggttgcgcgccccggggggtcggcgcgaactcgacgaggcggtcggagaaatcgccagcaccccgctgaaccgcgaccacccgctgtgggagatgtacttcgttgaggggcttgccaaccaccggatcgcggtggttgccaaaattcaccatgcgttggctgacggtgttgcctcggcaaacatgatggcacgggggatggatctgctgccgggaccggaggtcggccgctatgtgcctgaccccgctcctaccaagcggcagttgctgtccgcggcgttcatcgaccacttgcgccacctcggccggattcctgcaaccatccggtacaccacgcagggtctaggccgggtgcgacgtagctcgcgcaagctctcacccgcactgaccatgccatttaccccgccaccgacgttcatgaatcaccggctcaccccggagcgcaggttcgccaccgccaccctggcgctgattgacgtgaaggcgacggccaagttgctgggggcgacgatcaacgacatggtgctggccatgtcgaccggcgctctgcgtaccctgctattgcgctatgacggcaaggccgaaccgctgctggcgtcggtcccggtgagttacgacttctcaccggagcggatctccggtaaccgcttcaccggaatgctggtggcgctgcctgccgactccgacgacccgttgcagcgggtgcgcgtctgtcacgaaaacgcggtctccgccaaggagagccaccagcttttgggaccggagttgatcagccgctgggcggcttactggccacctgccggtgcggaagccttgttccggtggttgtctgagcgcgacgggcagaacaaggtactcaacttgaatatctcgaatgttcccggtccgcgcgaacgcggccgcgtgggggccgcgctggtcaccgagatctattcggtgggcccgttgaccgccggtagcggattgaatatcacggtgtggagttatgtcgatcagctcaatatctcggtgttaaccgatggttccaccgtgcaggacccgcatgaagtaaccgcgggaatgatcgcggacttcatcgaaatacgccgcgccgctggtctttccgtggagttgacagtcgtcgagtccgcgatggcgcaggca

SEQ ID NO: 36 - DNA, 786 bp, Mycobacteria tuburculosis
atgagcagcgaaagcgacgcagccaacaccgaacctgaggttctggtagaacagcgggatcggattttgatcatcacgatcaaccgcccgaaagccaagaacgcggtcaacgccgcagtcagccggggcttggccgatgcgatggatcagcttgacggcgatgccggcctgtcggtggcaatcctgaccggtgggggcggttcgttctgcgcgggcatggacctcaaggcgttcgcccggggcgagaatgtcgtcgtcgaaggtcgcggccttggctttaccgaacgtccgccgaccaagccgctcattgctgcggtggaaggctacgcgttggcgggtggcaccgagctggcgcttgctgccgacctgatcgtggcggccagggattcggcgttcgggattcctgaagtcaagcggggtctggttgccggcggcgggggattgctgcggttgccggagcgcatcccgtatgcgatagccatggagttggcgctgaccggtgacaacctaccggccgaacgcgcgcacgagctggggctcgtcaacgttttggccgagccggggaccgccctcgatgctgcgatcgcgttggcggagaagatcaccgccaatgggccgctggcggtggtggccaccaagcggattatcaccgagtcgcgtgggtggagtcccgacactatgttcgctgagcagatgaagatcctggtgccggtgttcacctccaacgacgcgaaggaaggtgcgatcgcgttcgccgagaggcgccggccccgttggacgggcacc

SEQ ID NO: 37 - DNA, 1461 bp, M. tuberculosis
atgtctgacagtgccacggaatacgacaagcttttcatcggcggcaagtggaccaaaccgtcgacctccgatgttatcgaggtacgctgcccagccactggggaatatgtcggcaaggtgccgatggcggccgccgccgacgtcgacgccgcggtcgccgcagcacgtgcggcgttcgacaacggcccctggccctcgaccccgccgcacgagcgtgcggcggtgatcgctgcggcggtcaagatgctggctgagcgcaaggacctgttcaccaagctgctcgcagccgaaaccggccagccgccgaccatcatcgagacgatgcactggatgggttcgatgggggcgatgaactactttgccggtgcagcggacaaggtcacctggaccgaaacccgcaccggctcctatggacagagcattgtcagccgtgagccggtcggtgtggtgggcgcgatcgtggcctggaacgtcccgctgtttctggccgtcaacaagattgcgccggcgctgctggccggctgcaccatcgtgctcaagcccgccgccgaaacaccgctgaccgcaaacgctttggcggaggtgttcgccgaggtgggcctgcccgagggggtgttgtcggtagtgccgggagggattgagaccggtcaggcgctgacgtctaacccggacatcgacatgtttaccttcaccggcagctcggccgtcggccgagaggtcggcaggcgtgccgctgagatgctcaagccgtgcaccttagaactcggcggcaagtcggcggccatcattctcgaggacgtcgacctggccgcagctattccgatgatggtgttctccggcgtcatgaacgccggacagggctgcgtcaaccagacccgcattctggctccgcgctcccggtacgacgaaatcgtggctgcggtaactaatttcgtaacggctctcccggtgggcccgccgtcggacccggcagctcagatcgggccgctgatctcggagaagcagcggactcgcgttgaaggctacatcgccaagggcatcgaggagggcgctcggttggtgtgcggcggcggccgtcccgagggcttggacaacggcttctttatccaacccaccgtattcgccgatgtcgacaacaagatgaccatcgcacaggaggagatcttcgggccggtgctggccatcattccttatgacaccgaggaggacgcgatcgcgatcgccaacgattcagtgtatgggctggcgggcagcgtgtggaccaccgacgtgcccaaaggcatcaagatctcgcagcagatccgcaccgggacatacggaatcaactggtacgccttcgatcccggctcacccttcggcggctacaagaactccggaatcggccgcgagaacgggcccgagggtgtcgaacacttcacccagcaaaagagtgtcctgctgccgatgggctacaccgtcgcg

SEQ ID NO: 38 - DNA, 831 bp, M. tuberculosis
atggcacgctgcgatgtcctggtctccgccgactgggctgagagcaatctgcacgcgccgaaggtcgttttcgtcgaagtggacgaggacaccagtgcatatgaccgtgaccatattgccggcgcgatcaagttggactggcgcaccgacctgcaggatccggtcaaacgtgacttcgtcgacgcccagcaattctccaagctgctgtccgagcgtggcatcgccaacgaggacacggtgatcctgtacggcggcaacaacaattggttcgccgcctacgcgtactggtatttcaagctctacggccatgagaaggtcaagttgctcgacggcggccgcaagaagtgggagctcgacggacgcccgctgtccagcgacccggtcagccggccggtgacctcctacaccgcctccccgccggataacacgattcgggcattccgcgacgaggtcctggcggccatcaacgtcaagaacctcatcgacgtgcgctctcccgacgagttctccggcaagatcctggcccccgcgcacctgccgcaggaacaaagccagcggcccggacacattcctggtgccatcaacgtgccgtggagcagggccgccaacgaggacggcaccttcaagtccgatgaggagttggccaagctttacgccgacgccggcctagacaacagcaaggaaacgattgcctactgccgaatcggggaacggtcctcgcacacctggttcgtgttgcgggaattactcggacaccaaaacgtcaagaactacgacggcagttggacagaatacggctccctggtgggcgccccgatcgagttgggaagc

SEQ ID NO: 39 - DNA, 300 bp, M. tuberculosis
atgtgctctggacccaagcaaggactgacattgccggccagcgtcgacctggaaaaagaaacggtgatcaccggccgcgtagtggacggtgacggccaggccgtgggcggcgcgttcgtgcggctgctggactcctccgacgagttcaccgcggaggtcgtcgcgtcggccaccggcgatttccggttcttcgccgcgcccggatcctggacgctgcgcgcgctgtcggcggccggcaacggcgacgcggtggtgcagccctcgggcgcgggcatccacgaggtagacgtcaagatcacc

SEQ ID NO: 40 - DNA, 441 bp, M. tuberculosis
atggccaatgtggtagctgaaggtgcctacccttactgtcggctcactgatcagccgctgagtgtggacgaagtgctagccgccgtctcgggccccgaacaaggcggcattgtcatatttgtgggaaacgtgcgtgaccacaatgccgggcatgatgtcacgcggttgttctacgaggcgtatccgccgatggtgattcggacattgatgtcgatcatcggacggtgtgaagacaaggccgagggtgtccgcgttgctgtcgcgcaccggaccggtgaattgcaaatcggtgatgccgcggtcgttattggcgcgtcagctccccaccgtgcggaggcatttgacgccgcgcgtatgtgtatcgagttgcttaagcaggaagtgccgatttggaagaaggaattcagctcgaccggtgctgaatgggtcggcgatagacca

SEQ ID NO: 41 - DNA, 600 bp, M. tuberculosis
atgagtccgtctccatcggccctgctcgccgaccacccggaccgcattcgttggaacgcgaaatacgagtgcgctgaccccacggaggcggtatttgcgcccatatcctggctcggcgacgtgctgcagttcggggtgccagaagggccggttctggaactggcgtgcggtcggtccggcaccgcgctggggctagccgcggcgggccgctgcgtgactgcgatcgacgtttccgataccgcgttggttcagctcgagctcgaagcgacccgacgggaattggccgatcgcctcacactggtgcacgccgatctctgctcctggcagtcgggggatggacgctttgctctggtactttgccgactattctggcatccgcccacttttcgccaggcttgcgaggctgtggcgccgggcggtgtagtggcgtgggaggcatggcggcggcccatcgatgtcgctcgggatacccgtcgagccgaatggtgcttgaagccaggccagcccgagtctgaacttcccgccggcttcacggtgattcgggtggtcgacaccgatggttcagagccgtcgcggcgcatcatcgcccaacggtcactg

SEQ ID NO: 42 - DNA, 1200 bp, Mycobacteria tuburculosis
atgacaagcacctcgattccgacgttcccgttcgaccggccggtcccgacggagccgtccccaatgctgtcggaactgagaaacagctgtccggtagccccgatagagttgccctcggggcacacagcatggctcgtcactcgctttgacgatgtaaagggagtgctgtccgacaagcgtttcagctgcagggcggcagcgcacccgtcgtcgcccccgttcgtgccgttcgtgcagctttgccccagcttgttgagcatcgatgggccccaacacaccgcggcccgccgtctgctcgcgcagggcctaaatcccggcttcatcgcacgcatgcggcccgttgtccaacagatcgtcgacaatgcgctcgacgatctggcagccgcggaaccaccggtggacttccaggaaatagtaagtgtccctatcggagaacagctcatggccaagctactcggggtcgagcccaaaaccgtgcacgagctcgcggcgcacgtggatgcggcgatgtccgtgtgtgagatcggcgacgaggaggtgagccggcggtggtcagcactgtgcacgatggtcatcgacatactgcaccgcaagctcgccgaaccgggtgatgacctacttagcacgatcgcccaggcgaaccggcaacagtccaccatgaccgacgagcaggttgtcggcatgctcctcaccgtcgtgatcggaggagtcgacacaccgatcgccgtgatcacaaacgggctggcgagcctgctgcaccaccgcgatcaatatgaacggctcgttgaagacccaggccgtgtcgctcgtgcggttgaagaaatagtccggtttaatccggcaactgaaattgagcacttgcgagttgtcaccgaggatgtcgtcattgccggaaccgcgctatcggcggggagcccagcatttacctctatcacttcggctaaccgcgactccgaccaattcctggaccccgatgagtttgatgtcgaacgtaatccgaacgaacacatagcatttggatatggtccacatgcttgcccggcctcagcgtattcacgcatgtgcttgacgacgttcttcacctcgcttacccagcgatttccgcaacttcaactcgcaagaccgtttgaggatttggaacgacggggtaagggcctacattcggtggggatcaaggaactccttgttacctggccgacg

SEQ ID NO: 43 - DNA, 498 bp, Mycobacteria tuburculosis
gtgcgcattgtcaatgcggcggacccattttcgatcaacgatctaggctgtggctatggggctctactggactacctagatgcgcgtggcttcaaaactgattacaccggcatcgacgtctcccccgaaatggtgcgcgcggccgcactacgtttcgaaggtcgggcgaacgcagacttcatctgcgcggcgcgcatagatcgggaggcggactatagcgtcgcgagtggaatattcaatgttcgtctgaaatcgttggacacggaatggtgcgctcacatcgaagcgacgctcgacatgctgaatgccgcgagtcgccgtggcttctcttttaattgcctgacatcttattccgatgcatcaaagatgcgcgacgacctgtactatgctgacccatgcgccctatttgatctctgcaagcgcaggtactccaagagtgttgcgcttctgcacgactacggcttgtatgaattcacaattctggttaggaaggcgtca

SEQ ID NO: 44 - DNA, 693 bp, Mycobacteria tuburculosis
ttgaagaaagtcgcgattgttcaatcaaattacataccttggcgaggatattttgacctgattgcattcgtcgatgaattcatcatctatgatgacatgcaatataccaagcgtgattggcgaaacagaaatcggatcaaaacgagccaggggttacagtggataactgttcccgtccaggtgaagggacgtttccatcaaaagatacgtgagacgctgatcgacggcaccgattgggcgaaagcgcactggcgggcactagaattcaactacagcgcggccgctcattttgcggagatcgctgactggctcgcgccgatttacctcgaagaacagcacacgaatctttccttactcaacaggcgtctattgaatgcgatttgcagttatctcggtatcagcacgcgactggcaaattcgtgggactacgaattagccgacggcaagaccgagagactggccaacctctgccaacaggccgcagcgaccgaatatgtctctggcccctcagcccgttcgtatgtcgatgagcgcgtgttcgacgaacttagcatccgggtaacttggttcgattatgacggctaccgcgattataagcaattgtggggagggttcgagcccgccgtgtcgattctggatctgctctttaacgtcggagccgaggctccggactatttgaggtactgtcgccag

SEQ ID NO: 45 - DNA, 395 bp, Mycobacteria tuburculosis
  misc_feature (1)...(395): n = A,T,C or G
vvmsartgvarhgtsgrgcgdvgargndvsvatrkrsrgdrvgnhgararrmkrvrgavtasrrwagssrtmgtasvsaatayaswyavdvstvvgdcwdwgmngrhcsdyamvaaagnadysadytavaawaaryagshwgargcyvatmavsawaargargrvvvtgaaaawgvdrgnstgvvaayvasrrwgattvavvkvvgvvaarwrwaggtgvvvsnaawrggtashgknssggrdrnvsgkadsknysgkgtgrtgavvvvvavagrrvmvgvatatsadvayyvvaavardnggagdaahgdrrravgvcvsavasvnvavgyvyggakgvvgttvttvtwawvtcvvvsyarkarhdshngtrsddtaasttscnvssrggcnyt

SEQ ID NO: 46 - DNA, 879 bp, Mycobacteria tuburculosis
gtgtttgcgttgagtaataatctgaaccgtgtgaacgcatgcatggatggattccttgcccgtatccgctcacatgttgatgcgcacgcgccagaattgcgttcactgttcgatacgatggcggccgaggcccgatttgcacgcgactggctgtccgaggacctcgcgcggttgcctgtcggtgcagcattgctggaagtgggcgggggggtacttctgctcagctgtcaactggcggcggagggatttgacatcaccgccatcgagccgacgggtgaaggttttggcaagttcagacagcttggcgacatcgtgctggaattggctgcagcacgacccaccatcgcgccatgcaaggcggaagactttatttccgagaagcggttcgacttcgccttctcgctgaatgtgatggagcacatcgaccttccggatgaggcagtcaggcgggtatcggaagtgctgaaaccgggggccagttaccacttcctgtgcccgaattacgtattcccgtacgaaccgcatttcaatatcccaacattcttcaccaaagagctgacatgccgggtgatgcgacatcgcatcgagggcaatacgggcatggatgacccgaagggagtctggcgttcgctcaactggattacggttcccaaggtgaaacgctttgcggcgaaggatgcgacgctgaccttgcgcttccaccgtgcaatgttggtatggatgctggaacgcgcgctgacggataaggaattcgctggtcgccgggcacaatggatggtcgctgctattcgctcggcggtgaaattgcgtgtgcatcatctggcaggctatgttcccgctacgctgcagcccatcatggatgtgcggctaacgaagagg

SEQ ID NO: 47 - DNA, 1296 bp, Mycobacteria tuburculosis
atgtacgagagacggcatgagcgcggaatgtgcgaccgtgccgtcgagatgaccgacgtcggcgctacggcagcccccaccggacctatcgcgcggggcagcgtcgctcgggtcggcgcggcgaccgcgttggccgttgcctgcgtctacacggtcatctatctggcggcccgcgacctacccccggcttgtttttcgatattcgcggtgttttggggggcgctcggcattgccaccggcgccacccacggcctcctgcaagaaacgacccgcgaggtccgctgggtgcgctccacccaaatagttgcgggccatcgtacccatccgctgcgggtggccgggatgattggcaccgtcgcggccgtcgtaattgcgggtagctcaccgctgtggagccgacagctattcgtcgaggggcgctggctgtccgtggggctactcagcgttggggtggccgggttctgcgcgcaggcgaccctgctgggcgcgctggccggcgtcgaccggtggacacagtacgggtcactgatggtgaccgacgcggtcatccggttggcggtcgccgcggcagcggttgtgatcggatggggtctggccgggtacttgtgggccgccaccgcgggagcggtggcgtggctgctcatgctgatggcctcgcccaccgcgcgcagcgcggccagcctgctgacgcccgggggaatcgccacgttcgtgcgcggtgccgctcattcgataaccgccgcgggtgccagcgcgattctggtaatgggtttcccagtgttgctcaaagtgacctccgaccagttaggggcaaagggcggagcggtcatcctggctgtgaccttgacgcgtgcgccgcttctggtcccactgagcgcgatgcaaggcaacctgatcgcgcatttcgtcgaccggcgcacccaacggcttcgggcgctgatcgcaccggcgctggtcgtcggcggcatcggtgcggtcgggatgttggccgcagggcttaccggtccctggttgctgcgtgttggattcggccccgactaccaaactggcggggcgttgctggcctggttgacggcagcggcggtagctatcgccatgctgacgctgaccggcgccgccgcggtcgcggccgcactgcaccgggcgtatttgctgggctgggtcagcgcgacggtggcgtcgacgctgttgctgctgctgccgatgccgctggagacgcgcaccgtgatcgcgctgttgttcggtccaacggtgggaatcgccatccatgtggccgcgttggcgcggcgacccgac

SEQ ID NO: 48 - DNA, 1020 bp, M. tuberculosis
gtgaagcgagcgctcatcaccggaatcaccggccaggacggctcgtatctcgccgaactgctgctggccaaggggtatgaggttcacgggctcatccggcgcgcttcgacgttcaacacctcgcggatcgatcacctctacgtcgacccgcaccaaccgggcgcgcggctgtttctgcactatggtgacctgatcgacggaacccggttggtgaccctgctgagcaccatcgaacccgacgaggtgtacaacctggcggcgcagtcacacgtgcgggtgagcttcgacgaacccgtgcacaccggtgacaccaccggcatgggatccatgcgactgctggaagccgttcggctctctcgggtgcactgccgcttctatcaggcgtcctcgtcggagatgttcggcgcctcgccgccaccgcagaacgagctgacgccgttctacccgcggtcaccgtatggcgccgccaaggtctattcgtactgggcgacccgcaattatcgcgaagcgtacggattgttcgccgttaacggcatcttgttcaatcacgaatcaccgcggcgcggtgagacgttcgtgacccgaaagatcaccagggccgtggcacgcatcaaggccggtatccagtccgaggtctatatgggcaatctggatgcggtccgcgactgggggtacgcgcccgaatacgtcgaaggcatgtggcggatgctgcagaccgacgagcccgacgacttcgttttggcgaccgggcgcggtttcaccgtgcgtgagttcgcgcgggccgcgttcgagcatgccggtttggactggcagcagtacgtgaaattcgaccaacgctatctgcggcccaccgaggtggattcgctgatcggcgacgcgaccaaggctgccgaattgctgggctggagggcttcggtgcacactgacgagttggctcggatcatggtcgacgcggacatggcggcgctggagtgcgaaggcaagccgtggatcgacaagccgatgatcgccggccggaca

SEQ ID NO: 4 - DNA, 966 bp, M. tuberculosis
atgaacgcgcacacctcggtcggcccgcttgaccgcgcggcccgggtctacatcgccgggcatcgcggcctggtcgggtccgcgctgctacgcacgtttgcgggcgcggggttcaccaacctgctggtgcggtcacgcgccgagcttgatctgacggatcgggccgcgacgttcgacttcgttctcgagtcgaggccgcaggtcgtcatcgacgcggcggcccgggtcggcggcatcctggccaacgacacctacccggccgatttcctgtcggaaaacctccagatccaggtcaacctgctggatgccgccgtggcggcgcgggtgccgcggctgctgttcctgggctcgtcgtgcatctacccgaaactcgccccgcagccgatcccggagagcgcgctgctcaccggtccgttggagccgaccaacgacgcgtacgcgatcgccaaaatcgccggcatccttgcggtccaggcggtgcgccgccaacatggcctgccgtggatctcggcgatgcccaccaacctgtacgggccaggcgacaacttttcgccgtccggctcgcatctgctgccggcactcatccgccgctatgacgaggccaaagccagtggcgcgcccaacgtgaccaactggggcaccggcacgccccgacgggagttgctgcacgtcgacgacctggcgagcgcatgcctgtatctgctggaacatttcgacgggccgacccatgtcaacgtgggaaccggcatcgaccacaccatcggcgagatcgccgagatggtcgcctcggcggtaggctatagcggcgaaacccgctgggatccaagcaaaccggacggaacaccacgcaaactgctggatgtttcggtgctacgggaggcgggatggcggccttcgatcgcgctgcgcgacggcatcgaggcgacggtggcgtggtatcgcgagcacgcgggaacggttcggcaa

SEQ ID NO: 50 - DNA, 729 bp, Mycobacteria tuberculosis
atgaggctggcccgtcgcgctcggaacatcttgcgtcgcaacggcatcgaggtgtcgcgctactttgccgaactggactgggaacgcaatttcttgcgccaactgcaatcgcatcgggtcagtgccgtgctcgatgtcggggccaattcggggcagtacgccaggggtctgcgcggcgcgggcttcgcgggccgcatcgtctcgttcgagccgctgcccgggccctttgccgtcttgcagcgcagcgcctccacggacccgttgtgggaatgccggcgctgtgcgctgggcgatgtcgatggaaccatctcgatcaacgtcgccggcaacgagggcgccagcagttccgtcttgccgatgttgaaacgacatcaggacgcctttccaccagccaactacgtgggcgcccaacgggtgccgatacatcgactcgattccgtggctgcagacgttctgcggcccaacgatattgcgttcttgaagatcgacgttcaaggattcgagaagcaggtgatcgcgggtggcgattcaacggtgcacgaccgatgcgtcggcatgcagctcgagctgtctttccagccgttgtacgagggtggcatgctcatccgcgaggcgctcgatctcgtggattcgttgggctttacgctctcgggattgcaacccggtttcaccgacccccgcaacggtcgaatgctgcaggccgatggcatcttcttccggggcagcgat

SEQ ID NO: 51 - DNA, 786 bp, Mycobacteria tuburculosis
gtgacgtctgctccgaccgtctcggtgataacgatctcgttcaacgacctcgacgggttgcagcgcacggtgaaaagtgtgcgggcgcaacgctaccggggacgcatcgagcacatcgtaatcgacggtggcagcggcgacgacgtggtggcatacctgtccgggtgtgaaccaggcttcgcgtattggcagtccgagcccgacggcgggcggtacgacgcgatgaaccagggcatcgcgcacgcatcgggtgatctgttgtggttcttgcactccgccgatcgtttttccgggcccgacgtggtagcccaggccgtggaggcgctatccggcaagggaccggtgtccgaattgtggggcttcgggatggatcgtctcgtcgggctcgatcgggtgcgcggcccgatacctttcagcctgcgcaaattcctggccggcaagcaggttgttccgcatcaagcatcgttcttcggatcatcgctggtggccaagatcggtggctacgaccttgatttcgggatcgccgccgaccaggaattcatattgcgggccgcgctggtatgcgagccggtcacgattcggtgtgtgctgtgcgagttcgacaccacgggcgtcggctcgcaccgggaaccaagcgcggtcttcggtgatctgcgccgcatgggcgaccttcatcgccgctacccgttcgggggaaggcgaatatcacatgcctacctacgcggccgggagttctacgcctacaacagtcgattctgggaaaacgtcttcacgcgaatgtcgaaa

SEQ ID NO: 52 - DNA, 894 bp, Mycobacteria tuburculosis
atgtcgacaaacccaggaccagccgaaggggctaaccaagtgatggcacaggaacattcggccggcgcggtacaattcaccgcccacaacgttcgcctcgacgacggaaccttgacgataccggagtcctcgcgcacgttagacgaatcgtcctggttcatctcggcgcgcgggattctggaaaccgtctttcccggggacaagagccacctacgcctggccgatgtcggctgcttggaaggcgggtacgcggtcgggttcgcgcgcatgggatttcaggtcctcgggatcgaggttcgcgagctgaacatggcggcctgcaactacatcaaatcgaagaccaacctgccgaatctccggttcgtccacgacaacgccctcaacatcgccaaccacgggctcttcgataccgtcttctgctgcggcctcttctaccacctggagaatccgaagcaatacctggaaaccctctcgtcggtaacgaacaagctgctgattctccagacgcacttctcgatcatcaaccggagcgataaatggctccggttgcccacgacggcacgacaattgaccgatcggttgctgcggcggccggcgccggtgaagttcatgctctcggcgcccaccgaacatgagggacttcccggtaggtggtttaccgagttttccgacgaccgctcgtttggccagcgcgacaccgcaaaatgggcgtcctgggacaatcgccggtcattctggattcaacgcgagcacctacttcaggccatcaaagacgtcggcgtcgacctggtgatggaggagtacgacaacttggaaccaagcatcgccgagtcgttgctcggaggttcctatgcggcgaatcttcgaggcaccttcatcggtatcaagacccgg

SEQ ID NO: 53 - DNA, 1119 bp, Mycobacteria tuburculosis
gtgccgtacgtccgccgaccaccaggccacgacggccgacggccggcgggcacaggcgattcacgttcgccatcgcaatacccttgcggccgcgcaggaaaagggccgacggtgagtccccagctttgccccaaggtgagcatcgtctcgaccactcacaaccaggcgggctacgcccgtcaggccttcgacagctttctcgaccagcaaaccgacttcccggtggagatcatcgtcgccgacgacgcgtcgaccgatgccaccccggcgatcatccgtgagtacgccgagcggtacccgcacgtgttccggccgatcttcaggaccgaaaacctcggcctcaatgggaacctgaccggcgccctgtcggccgctcgcggcgagtacgtcgcgttgtgcgaggcggacgactactggatcgatccgctgaagctaagcaaacaggtcgcattcctcgaccggcaccccaagacgacggtgtgcttccatcccgtccgagtgatatgggaggacggccatgccaaggactcgaagttccccccggttcgggtgcggggcaacttgagcctggatgcgttgatcttgatgaacttcatccagaccaactcggccgtgtaccgtcgcctcgagcgctacgacgacattcctgccgacgtcatgcccctggactggtatctgcacgtccggcacgcggtgcatggcgacatcgccatgttgcccgacaccatggccgtgtatcgccgccacgcccaaggcatgtggcacaaccaggtggtggacccgccaaagttctggttgacgcagggtccggggcatgcggcgacgtttgacgcgatgctcgacctgttcccgggagaccccgcgcgcgaggagctcatcgccgtcatggccgactggatccttcgccagatcgccaacgttccaggcccggaggggcgcgccgcgctgcaggaaaccatcgcgcgccatccccggatcgccatgctggcgctgcagcaccgcggggcgacacccgcgcggcggctcaagacccagtggcgcaagctcgccgccgcgacgccgagccgcagggggctcgtggatgtgtggccctcccggctccgacgcggctgtcgagcc

SEQ ID NO: 5 - DNA, 282 bp, Mycobacteria tuburculosis
atgaccatcaactatcagttcggtgatgtcgacgctcatggcgccatgatccgcgctcaggccgggttgctggaggcggagcatcaggccatcgttcgtgatgtgttggccgcgggtgacttttggggcggcgccggttcggtggcttgccaggagttcatcacccagctgggccgtaacttccaggtgatctacgagcaggccaacgcccacgggcagaaggtgcaggctgccggcaacaacatggcacaaaccgacagcgccgtcggctccagctgggcc

SEQ ID NO: 55 - DNA, 294 bp, Mycobacteria tuburculosis
atggcaacacgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatggcccagatgaatcaggcgtttcgcaacatcgtgaacatgctgcacggggtgcgtgacgggctggttcgcgacgccaacaactacgagcagcaagagcaggcctcccagcagatcctcagcagc

SEQ ID NO: 56 - DNA, 324 bp, Mycobacteria tuburculosis
gtgcttttgcctcttggtccgcctttgccgcccgacgcggtggtggcgaaacgggctgagtcgggaatgctcggcgggttgtcggttccgctcagctggggagtggctgtgccacccgatgattatgaccactgggcgcctgcgccggaggacggcgccgatgtcgatgtccaggcggccgaaggggcggacgcagaggccgcggccatggacgagtgggatgagtggcaggcgtggaacgagtgggtggcggagaacgctgaaccccgctttgaggtgccacggagtagcagcagcgtgattccgcattctccggcggccggc

SEQ ID NO: 57 - DNA, 1524 bp, M. tuberculosis
atgtcacgccgagcattcctggctaaggcggctggagccggggcagcggcggttttgacggactgggccgcaccggtgatcgaaaaggcctatggtgccggtccctgctcgggtcatttgaccgatatcgagcacatcgtgctgtgcctacaggagaacaggtcgttcgatcactatttcggcacgctttctgccgtcgacgggttcgacactccgacgccgctgtttcaacaaaagggctggaacccggagacgcaggcgctggaccccaccggcattacgctgccctaccgcatcaataccaccgggggtcccaacggggttggcgagtgcgtcaacgacccagaccaccagtggattgccgcgcacttgtcatggaacggcggcgccaatgacggctggctgccggcgcaggcgcggacccggtcggtggccaacacgcccgtggtgatgggctattacgcacgtcctgacataccgatccactacttgttggccgataccttcacgatctgcgaccagtacttctcgtcgcttcttggcgggacgatgcctaaccggctctattggatcagcgccaccgtcaatcccgacggggatcaaggtgggccgcagatcgtcgaacccgccatccagccgaagttgaccttcacctggcgcatcatgccgcagaacctcagtgacgccggcatcagttggaaggtgtacaacagcaagctgctcggcgggctcaacgacacttccttgagccgtaacgggtatgtgggcagtttcaaacaggccgcagatccgaggtcggacctggcccgttatggcatcgccccggcctacccgtgggatttcatccgcgacgtcatcaacaacacgctgccccaggtgtcctgggtcgttccgttgaccgtcgagtccgaacatccgtcattcccggtggcagtcggtgcggtgacgatcgtgaacttgataagggtgttgctgcgcaatccggcggtgtgggagaaaaccgcgttgatcatcgcctatgacgaacatggcggcttcttcgaccacgtcacaccgctcaccgcgccggagggcacacccggcgaatggattcccaacagtgttgacatcgacaaggtcgacggctccggcggaatacgtggacccatcggcttgggctttcgcgtgccctgcttcgtcatttcgccttacagtcgcggcgggctgatggtccatgatcggttcgaccacacatcgcagctgcaattgatcggcaagcgtttcggggtgccggttcccaacttgacaccctggcgtgccagtgtcaccggcgatatgacgtcggcattcaatttcgcggccccgccggacccgtcgccacccaatctggaccacccggtccgtcaattgccgaaggtcgccaagtgcgtgcccaatgtggtgctgggtttcttgaacgaaggcctgccgtatcgggtgccctacccccaaacaacgccagtccaggaatccggtcccgcgcggccgattcccagcggcatctgc

SEQ ID NO: 58 - DNA, 1536 bp, M. tuberculosis
atgtcacgtcgagagtttttgacaaagctcactggcgcaggcgcagcggcattcctgatggactgggctgcaccggtgattgaaaaggcctacggcgccgggccttgtcccggacatttgaccgacatcgagcatatcgtgttgctgatgcaggagaaccggtcattcgaccactatttcggaacgctttccagcaccaatgggttcaacgccgcgtcgccggcattccaacaaatgggttggaaccccatgacgcaggcgttggaccccgccggggtcaccattccgttccgcttggacaccacccgaggccccttcctggacggcgagtgcgtcaacgaccccgagcaccagtgggtggggatgcacctggcctggaacggtggtgccaacgacaactggctgccggcgcaggcgaccacccgcgcaggaccatatgtccctttgaccatgggttactacacgcgccaagacatcccgatccactatctgctggccgacacgttcaccatctgcgacggctaccattgctcgctgctgacgggcaccctgcccaaccggctctactggttgagcgccaacatcgaccccgccggcaccgacgggggaccccaattggtagagccgggcttcctgccgctgcagcaattcagttggcgcatcatgccggaaaacctcgaagatgccggggtcagctggaaggtgtaccagaacaagggcctcgggcgattcatcaacacgcccatcagcaataacgggctggtgcaggccttccgccaggcagctgatccgaggtcgaacttggcccgctacggtatcgccccgacctaccctggggacttcgctgccgacgtcagggccaaccggctacccaaggtctcctggttagttcccaacatcctgcagtccgaacaccccgccctgccggtagcgcttggcgcggtgtccatggtgaccgcgctgcggatcttgctgtccaatcccgcggtgtgggaaaagaccgcacttatcgtcagctatgacgagaacggcggcttcttcgaccacgtcacgccccccacggcaccgcccgggacacccggcgaattcgtcacggtgcccaacatcgacgcagtacccgggtccggtggcattcgtggtccgctcggtctgggttttcgcgttccctgcattgtcatttcgccgtacagccgcggcccgctgatggtctccgacacgttcgaccacacctcgcaattgaagttgattcgcgcccggttcggcgtgccggttcccaacatgaccgcctggcgcgacggcgtggttggcgacatgacctcagcgttcaactttgcgactccaccgaattcgaccagacccaacttgagccacccgttgctgggagcgctgccgaagctgccgcagtgcatccctaacgtggtgttgggaaccaccgacggcgcgttgccgagcattccctatcgggtgccctatccgcaggtgatgccaactcaggaaaccacacccgtccgcgggactcccagcgggctgtgcagc

SEQ ID NO: 59 - DNA, 1536 bp, M. tuberculosis; identical to SEQ ID NO: 58

SEQ ID NO: 60 - DNA, 1173 bp, Mycobacteria tuburculosis
atgattttggatttttcgtggttgccgccggagatcaactcggcgcggatctatgccggtgcggggtcggggccgttgtttatggcggcggcggcgtgggaggggttggctgcggatttgcgggcctcggcgtcctcgtttgatgcggtgatcgccgggttggcggctgggccgtggtcgggtccggcgtcggtggcgatggcgggggcggcggcgccgtatgtggggtggttgagtgcggcggccgggcaggcggagttgtcggctggtcaggctaccgcggcggcgacggcgtttgaggcggcgttggcggccacggtgcatccggcggcggtgacggcgaatcgggtgttgttgggggcgttggtggcgacgaacattttgggtcagaacacgccggcgattgcggccactgagttcgattatgtggagatgtgggctcaggacgtgggtgcgatggtggggtatcacgcgggggcggcggcggtggctgagacgttgacgccgtttagtgtgccgccgctggatttggcggggttggcttcccaggccggtgcgcagttgaccgggatggcgacgtcggtttcggctgcgttgtctccgatcgcggagggtgcggtggagggggtgccggctgtggtggctgcggcgcagtcggtggcggcggggttgccggtggatgcggcgctgcaggtggggcaggccgcggcgtatccggccagtatgttgattgggccgatgatgcagttggcgcagatggggactacggccaacacggctgggttggccggtgcggaggctgcggggttggctgcggcggatgtgccgacgtttgccggtgatatcgcttcggggacgggcctaggtggtgccggtggtctgggtgcggggatgtcggcggagttgggtaaggcgcggttggtgggggcgatgtcggtgcctccgacctgggaggggtcggttcctgcgcggatggccagttcggcgatggcgggtttgggggctatgcctgctgaggtgccggcggcaggcgggcccatggggatgatgccgatgccgatgggtatggggggtgctggggcgggtatgccggccgggatgatgggccgcggtggcgcaaatccgcatgtggtgcaggctcggcccagtgtggtgccgcgggtcgggatcgga

SEQ ID NO: 61 - DNA, 1062 bp, Mycobacteria tuburculosis
atgccggggcggttcagaaacttcggtagccaaaacctgggtagcggcaacatcggcagcaccaacgtgggcagcggcaacatcggcagcaccaacgtgggcagcggcaacatcggcgacacgaacttcggtaacggaaacaacggcaacttcaactttggtagcggcaataccggcagtaacaacatcggcttcggaaacaccggcagcgggaatttcggtttcggaaacacgggcaacaacaacatcggtatcgggctcaccggcgatggtcagatcggcatcggcggactgaactcgggcagcggaaacatcggtttcgggaactccggcaccggaaacgtcggtttgttcaactccggcaccggcaacgtaggcttcgggaactccggtactgcgaacactggattcgggaacgcgggcaacgtcaacaccggattttggaacggcggcagcacaaacactggcctcgctaacgccggcgccggcaacacaggctttttcgacgctggcaactacaacttcggcagtcttaacgccggaaacataaactcgagttttgggaattcgggtgacggcaacagtggtttcctcaatgctggcgacgtcaactccggtgtgggcaatgcgggtgatgtcaacactggcttagggaactcgggcaacatcaatactggtgggtttaatccgggcacgctcaacacgggcttcttcagcgcgatgacccaagctggtccgaattcgggcttcttcaacgccggtaccggtaactctggtttcgggcacaacgacccggctggcagtggcaactcgggcattcagaactcgggcttcggcaactcgggctatgtcaataccagcaccacaagcatgttcggcggtaactcaggggtgctcaacacgggctacggcaactcaggtttctataacgcggccgtcaacaacaccgggatttttgtgaccggcgtgatgagttcgggatttttcaattttgggacgggcaactcgggcctgctggtcagcggcaatgggctttcgggtttcttcaagaacttgttcgga

SEQ ID NO: 62 - DNA, 654 bp, Mycobacteria tuburculosis
atgagccgactcctagctttgctgtgcgctgcggtatgcacgggctgcgttgctgtggttctcgcgccagtgagcctggccgtcgtcaacccgtggttcgcgaactcggtcggcaatgccactcaggtggtttcggtggtgggaaccggcggttcgacggccaagatggatgtctaccaacgcaccgccgccggctggcagccgctcaagaccggtatcaccacccatatcggttcggcgggcatggcgccggaagccaagagcggatatccggccactccgatgggggtttacagcctggactccgcttttggcaccgcgccgaatcccggtggcgggttgccgtatacccaagtcggacccaatcactggtggagtggcgacgacaatagccccacctttaactccatgcaggtctgtcagaagtcccagtgcccgttcagcacggccgacagcgagaacctgcaaatcccgcagtacaagcattcggtcgtgatgggcgtcaacaaggccaaggtcccaggcaaaggctccgcgttcttctttcacaccaccgacggcgggcccaccgcgggttgtgtggcgatcgacgatgccacgctggtgcagatcatccgttggctgcggcctggtgcggtgatcgcgatcgccaag

SEQ ID NO: 63 - DNA, 489 bp, Mycobacteria tuburculosis
gtgtgctgcaatggcgtggtgactccgggtgatccagccgacattgcagcgatcaaacagctcaaataccggtatctgcgggcattggacaccaagcattgggacgacttcaccgacaccctggccgaggatgtcaccggcgattacgggtcatcggtcggtacggagctgcacttcaccaaccgcgccgacctggtcgactacctgcgccaggcactcggcccgggtgtcatcaccgaacaccgggtcacccatccggaaatcaccgtgaccggcgataccgcaaccggcatctggtacctgcaagaccgggtcatcgtcgccgagttcaatttcatgctcatcggcgccgcgttctaccacgaccagtaccgacgaaccaccgacggctggcggatcagcgccaccggctacgaccgaacctacgaggcgaccatgtcgttggcgggccttaacttcaacatcaggccgggccgcgcgctggccgat

SEQ ID NO: 64 - DNA, 1227 bp, Mycobacteria tuburculosis
atgagccaatcccggtacgcggggttgtcccgcagcgagctggcagttctgttacccgagctgttgttgatcggccagctgatcgaccgatcgggcatggcctggtgtatacaggcattcggccgccaggagatgctgcagatcgccatcgaggagtgggcgggcgccagcccgatctacaccaagcgcatgcaaaaggcgctgaacttcgagggcgacgacgtgcccaccatcttcaaggggctacagctcgacatcggcgcgccgccgcaattcatggacttccgtttcaccctgcacgaccgctggcacggcgagtttcacctcgaccactgcggtgcgctgctcgacgtggagccgatgggcgacgactacgtcgtcggcatgtgccacaccatcgaagatccgacgttcgacgccaccgcgatcgcgaccaacccgcgcgcgcaggtgcgccccatccaccggccgccccgcaagccggccgaccggcatccgcactgtgcgtggaccgtcatcatcgacgagtcctatcccgaggctgagggtattccggcgctggacgcggtccgtgaaaccaaagctgccacctgggaattagacaacgtcgatgcgtctgacgacgggctggtggactattcgggtccgctggtgtccgacctggacttcggggcgttctcgcattccgcactggtgcggatggccgatgaggtctgcctgcaaatgcacctgctgaatctgtcgttcgccattgccgtgcggaaacgggccaaagccgatgctcaactggccatttcggtgaacacccgccagttgatcggagtggccgggctgggcgcagaacgcattcaccgtgcgatggctttacccggcggaatcgaaggcgcgttaggtgtgctggagctacacccgctgctcaacccggccggttacgtgctggccgaaacgtcgccggaccgtctggtggtgcacaactcgccagcccacgccgacggcgcctggatttcgttgtgcacaccggcatccgtgcagccgttgcaggccatcgccaccgctgtagacccgcatctgaaggttcggatcagcgggacggacaccgactggaccgcggaactcatcgaggccgatgccccagcgagcgaactgccggaggtgttggtagccaaggtcagtcgcggatcggtcttccagttcgagccgaggcgctcactgccgttgaccgtgaaa

SEQ ID NO: 65 - DNA, 1860 bp, Mycobacteria tuburculosis
atgtacgacccgctggggttgtcgatcgggaccacaaacctggtcgcggcgggtaacggaggtccgccggttactcgtcgcgccgtgctgaccctgtacccgcattgcgcaccgaaaatcggtgtgcctagccagaacccgaacttgatcgagccgggcgccctaatgagcggctttgttgagcgcattggagatgcggtggcgctggtgtctcccgacggatccgtgcacgatccagacctcttgctggtcgaggcgctggatgcgatggtgctgaccgccggtgcggacgcgagttcctcggagatcgccattgccgttcccgcgcattggaagcccggagctgtacacgcactgcgtaacggtttgcggacgcacgtcggcttcgtccgcagcggcatggcgccgcgcctggtttccgatgcgatcgcggcgttgaccgcggtgaactcggaattgggcctgccccacggcagtgtggtggggttgcttgatttcggtggctccgcgacttacgtcaccttggtggagaccaagtcggattccaggacgtcggatttccagcccgttagtgccacggcacggtaccaggacttttccggtagtcagatcgaccaggctttgctgcttcgggtcatcgaccaattcgggtacggcgatgacgtcgatccggccagtaccgccgcggtcgggcaactcggccaactcagggagcagtgccgtgcggcaaaggaacgactgtccaccgacgttgccacggaattgttcgctgagcttgccgggtgcagctcgagcatcgagatgactcgggaacagctcgaagacctgatccaggatccattgaccggcttcatctacgcgttcgacgacatgctggcgcgccacaacgcgagctgggcggatctcgcggcggtggtcaccgtcggcggtggtgccaatattccccttgtgactcaacgtctttcgttccacactcgtcgacctgtgctgaccgcgtcgcaacccgggtgcgcggcggcgatgggtgcgttgctgctcgccaaccgtgggggagagcgcgattcgcgaacgcggacgtccatcggcctcgccacggccgcagccgccggcaccagtgtcatcgagctgccggccggcgacgtcatggtcatcgaccatgaggccttgaccgatcgcgagttggcctggtcgcagaccgacttcccaagcgaagctccggcgcgtttcgagggcgactcgtataacgaaggcggcccctgctggtcgatgcgtctgaacgcggtcgagccccccaaaggaccagcgtggcggcgaatccgggtgtcgcagttgctcatcggggtgtcggcggtagtggccatgaccgcgatcgggggcgtggcattgacgttgacagccatcgagagacgcccaagcccgctaccaaccccaattgtgcccggcctggccccgatgccgcccggatccgtcgtgcctagctcgcgcgcaccgaccccgccgccaccgccgtcgaccgttgcgccgcttcccagtgcggcaccggccccgacgacggtcgcgccggcaccgccgccgcccacacaggtggtgacgaccacgacagcgccacccgtcaccacgacgccgaggccgtcgccgaccaccacaacgaccaccgcgccaccgtcgacaacgacgacaaccgagccgccggtgacgaccacttcgacgattccaacgattccgacgactacgacgacggtgaagatgaccacggagtggttgcacgtcccgtttttgcccgttccgatcccggtcccgattccgcaaaatccgggtgccggcgaaccgcagaacccgttcggaagccttggctctggg

SEQ ID NO: 66 - DNA, 720 bp, M. tuberculosis
atgatccgattggtccgtcattcgatcgccctggtggccgccggccttgccgccgcattgtcggggtgcgattcccacaactcgggatcgctcggtgccgatccgcggcaggtgaccgtgttcggatccgggcaagtgcagggtgtgccggacacgttgatcgctgacgtcggcattcaggtcaccgcggccgacgtcaccagcgcgatgaaccagaccaatgatcgccagcaagcggtgatcgatgcactggtgggtgccggcctggaccgcaaggacatccgcaccaccagggtcaccgtggcaccgcagtacagcaatccggagccggccggaaccgccaccatcaccgggtatcgggcagacaacgacatcgaggtgaagatccacccgaccgacgccgcgtcgcggctgctggccctcgtcgtcagcaccggcggtgacgccacccggatcagctcggtcagctactcgattggcgacgactcgcagctggtgaaggatgcccgggcgcgcgccttccaagacgccaagaaccgtgcggaccagtacgcacaactgtcggggctgcggctaggcaaggtgatctcgatctccgaggcatctggcgccgcgcccacgcacgaggcgccggcgccgccgcgcggcctatccgcggtgccgctggaacccggccagcagacggtgggcttctcggtcacggtggtctgggaactgacc

SEQ ID NO: 67 - DNA, 297 bp, Mycobacteria tuburculosis
atgtcgatcatgcacgccgagccagagatgctggctgcgaccgcgggggaactgcagtcgatcaacgccgttgcgcgggccggaaatgcagcggtggcgggcccgacgacgggtgtggttccggccgccgctgatttggtgtccctgctaaccgcctcccagtttgccgcgcatgcacagctgtaccaggcgattagtgccgaggcgatggcggtccaggagcagttggcgaccacgctgggcatcagcgccggttcatatgcggccaccgaggctgccaacgccgccacgatcgct

SEQ ID NO: 68 - DNA, 1239 bp, Mycobacteria tuburculosis
atgctggactttgctcagttaccgccggaggtcaactccgcgctgatgtacgccggacccggttcgggaccgatgctggctgccgcggcggcctgggaggcgctggccgccgagttgcaaaccacggcgtccacctacgacgctctgatcactggcctggccgacgggccatggcaggggtcctccgcggcgtccatggtggctgccgccacgccccaggtggcgtggttgaggagcaccgccgggcaggccgagcaagccggcagccaagcggtggcagcggcgagtgcttatgaggcggcgtttttcgcgaccgtgccgcccccggagatcgcggccaacagggcgttgttgatggcgttgctggcgacgaacttccttggccagaacacggcggcgatcgcggccaccgaggcgcaatacgccgagatgtgggcccaggatgcggccgcgatgtacggctatgctggcgcgtcggcggcggcgacgcagttgtcgccattcaatccggcggcgcagaccatcaacccggccgggctggccagccaggccgcatctgtcggacaagctgtcagcggggccgcaaatgcgcaagcactcaccgacattcctaaagcgttgtttgggcttagcggaatcttcaccaatgaaccgccttggctcaccgaccttggcaaggcgctcggtttgaccgggcacacctggtcctcggacggtagcgggctcatcgtgggcggagtgcttggcgactttgtgcagggtgtgaccgggtcggccgaacttgatgccagcgtggccatggacacgttcggcaaatgggtctcgcccgctcggctcatggtcacccaattcaaggactactttggcctggcgcacgacctgccgaagtgggcgagtgaaggcgccaaagccgccggtgaggccgccaaggcgttgccggccgccgttccggccattccgagtgctggcctgagcggcgttgcgggcgccgtcggtcaggcggcgtcggtcgggggattgaaggttccggccgtttggaccgccacgaccccggcggcgagccccgcggtgctggcggcgtccaacggcctcggagccgcggccgccgctgaaggttcgacacacgcgtttggcgggatgccgctcatgggtagcggtgccggacgtgcgtttaacaacttcgctgcccctcgatacggattcaagccgaccgtgatcgcccaaccgccggctggcgga

SEQ ID NO: 69 - DNA, 294 bp, Mycobacteria tuburculosis
atgacctcgcgttttatgacggatccgcacgcgatgcgggacatggcgggccgttttgaggtgcacgcccagacggtggaggacgaggctcgccggatgtgggcgtccgcgcaaaacatttccggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagc

SEQ ID NO: 70 - DNA, 282 bp, Mycobacteria tuburculosis
atgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggcc

SEQ ID NO: 71 - DNA, 1185 bp, Mycobacteria tuburculosis
atgaaggcaccgttgcgttttggcgttttcatcacgccattccatccgaccggtcaatccccgaccgtggcgttgcaatacgacatggagcgcgtcgttgcgctggaccggctcggctacgacgaggcgtggtttggcgaacaccactccggtggctacgagctgatcgcttgcccggaggtgtttatcgcggccgcagcggaacggaccacccacatccggctaggtaccggagtggtttcgctgccctaccatcatccgctaatggtggccgaccgttgggtgctgctggatcacctgacccgtgggcgggtcatgttcggcaccggccccggcgcgctgccgtcggacgcctacatgatgggcatcgatccggtcgagcagcgacgaatgatgcaggagtccctcgaggcgattctcgcgctgttccgtgccgcacctgacgagcgaatcgaccgccactccgactggttcaccctgcgtgaagcgcaattgcacatccgcccctacacctggccgtaccccgaaatcgctaccgcagccatgatttcgccatcgggtccgcgactggccggtgcgctgggcacgtcgctgttatcactgtcgatgtcagtgcccggcggctacgctgcgctggaaacagcgtggggcgtggtgcgggagcaggccgccaaagctgggcggggcgagccggatcgcgccgattggcgggtgttgagcatcatgcacttgtcggacagccgcgaccaggcgatcgacgactgcacttacgggttacccgacttctcgaggtacttcggcgcggcagggtttgtcccgttggcgaacaccgtggaaggcacccagtcgtctcgggaattcgtcgagcaatacgcggccaagggaaattgctgcatcggcacgcccgatgacgcgatcgcccacattgaagacttgctgcaccggtcgggtggcttcggaacgttgctactgctcggccacgactgggccccgccaccggcaacctttcactcctatgagctgttcgcccgtgctgtgattccttatttcaagggacaactcgcggcgccgcgggcgtcgcacgaatgggctagaggcaagcgcgaccaattgattggccgcgccggcgaagcggtcgtcaaagccatcaccgagcacgtcgccgaacaaggggaagcgggcagc

SEQ ID NO: 72 - DNA, 966 bp, M. tuberculosis
atgggcgcacctaccgaacggttagttgataccaacggcgtgcgactgcgagtggtcgaggccggtgagcccggcgcacccgtggtgatactggcccacggctttcccgaactggcctattcatggagacaccagattcctgcgcttgccgacgccggctaccacgtgttggctcccgatcagcgcggttacggcggatcgtctcgcccagaggcgatcgaggcctacgacattcaccggttgaccgctgacctagtgggcctactagatgatgtcggtgccgagcgggcggtctgggttggtcatgactggggtgccgtggtggtgtggaacgcgccactgctgcacgctgaccgagtcgccgccgttgccgcgttgagcgtccccgcgctgccccgggcacaggtgccgccgacgcaagcgttccgcagcaggtttggggagaacttcttctacatcctttatttccaggagcccggcatcgccgacgccgaactcaatggcgacccggcccgcacgatgcgccgaatgatcggcggtctgcgccctccgggcgatcagagcgcggcaatgcgtatgctggcgcccggccccgacggctttatcgatcggcttccggagccggccgggttgccggcctggattagtcaggaggaactcgaccactacatcggcgagttcacccgcaccggtttcaccggcggcctgaactggtaccgcaacttcgaccgcaactgggagaccacggccgacctcgccggcaagacgatctccgtgccctcgttgttcattgcgggcacagccgatcccgtcttgacgttcacccgcaccgaccgcgctgcggaggtgatctccggcccgtatcgcgaggtgctgatcgacggggccggtcactggctgcagcaggaacgtcccggtgaggtgaccgcggccctgctggagttcctgacggggttggagttgcga

SEQ ID NO: 73 - DNA, 1365 bp, Mycobacteria tuburculosis
gtgaataccgatgtgctggctggcctgatggccgagctgcccgaggggatggtggtcaccgaccccgccgtcaccgacggctaccggcaagaccgggcctttgacccttcggccggcaaaccgctggcaatcatccggccacggcgcaccgaagaggtgcagacggtgctgcgttgggccagtgcgaaccaggtgcccgtggtgacccgaggagccggtagcggcctttcgggcggggcgaccgccctggatggcgggatcgtgctgtccaccgaaaagatgcgcgacatcaccgtcgacccggtcacccgcaccgcagtgtgccagcccggcctgtacaacgccgaggtgaaggaggccgccgccgaacacggcctgtggtatcccccggatccgtcgtcgttcgagatctgcagcatcggcggcaacatcgccaccaacgccggcgggctgtgctgcgtgaagtacggcgtcacaggcgactacgtactgggcatgcaggttgtgctggccaacggcaccgcggtccggctgggcggcccacggctcaaggacgtcgccgggctttccctgaccaaactgttcgtcggcagcgaaggcacgctgggcgtcatcacggaggtgacgttgcgactgctgcccgcacagaatgcatcgagcatcgtggtggccagcttcggctcggtgcaggcggcggtcgatgcggtgctcggggttaccggccgacttcgccccgcgatgctggagttcatggattcggtggcgatcaacgccgtcgaggacaccttgcggatggacctggaccgcgatgcggcggccatgctggtggctggttctgatgaacgtggccgcgcggccaccgaagacgccgccgtgatggccgccgtgttcgccgaaaacggtgcgatagacgtgttttcgaccgacgacccggatgagggcgaggcgttcattgcggcccggcggttcgccattccggcggtcgagagcaagggggcgttgctgctcgaggacgtcggggtaccgctgcccgcactgggcgaactggtcaccgggattgcgcgcatcgccgaggagcggaatctgatgatctcggtgatcgcccacgccggggacggcaatacccacccgttgctggtgtacgaccccgcagatgccgcgatgctagagcgcgcccacctcgcgtacggcgaaatcatggacctggccgtcggcctgggcggcacgatcaccggcgaacacggcgtgggccggttgaaacggccgtggttggccggctatctcgggcccgacgtcctggccctcaaccagcgcatcaagcaagcgctggacccccagggcatcctcaatcccggctcggcgatc

SEQ ID NO: 74 - DNA, 1215 bp, Mycobacteria tuburculosis
atgacatcagtaatgtctcacgaattccagctcgccaccgccgaaacctggccgaatccgtggccgatgtaccgcgcgttgcgcgaccacgacccggtgcaccacgtcgtcccgccgcagcgtcccgagtacgactactacgtgctgtcccggcacgccgacgtctggtcggcagcgcgggaccatcagacgttctcgtcggcgcaaggcttgaccgttaactacggcgagctggaaatgattggactgcacgacaccccgcccatggtgatgcaggatccgccggtccacaccgagtttcgcaagctggtgtcgcgcggcttcacgccacgacaggtcgaaaccgtcgagcccacggtgcgcaagttcgtcgttgagcggctcgaaaagctgcgcgccaacggtggcggcgacattgtcaccgaactattcaaaccgctcccgtcgatggtggtggcgcactatctcggtgttcccgaagaggattggacgcaattcgacgggtggacccaggccatcgtggcggcgaacgcggttgacggcgccaccaccggcgcactggacgcggtcggctcgatgatggcctacttcaccgggctgatcgagcgacgccgcaccgagcccgccgacgacgccatctcccacctggtagccgccggggtcggcgccgacggcgacaccgccggcacactgtccatactggcgttcacgttcaccatggtcaccggcggcaacgacaccgtcaccggcatgctaggcggttcgatgccgttgctgcaccggcggcccgaccagcgccggctgctgctggatgacccagagggcatccccgacgcggtcgaggagctgctgcggctcacctcgccggtgcaggggctggcgcgcacaaccacgcgcgacgtcacgatcggtgacaccaccatcccggccggtcgccgggtgctgctgctgtacggctcggccaaccgtgacgaacgccaatacggcccggacgcagccgaactcgatgtcactcggtgcccgcgcaacatcttgaccttcagccacggcgcccaccactgcctgggtgcggccgcggcccggatgcaatgccgggtggcgctgaccgaactgctggcccggtgcccggacttcgaggtggccgagtcacgcatcgtgtggtccggcggcagttatgtccggcgtccgctgtcggtgccgttccgagtgacatcc

SEQ ID NO: 75 - DNA, 606 bp, Mycobacteria tuburculosis
atggcgggtaccgactggctgtccgcgcgtcggaccgagttagccgcagatcggatactcgacgccgccgagcgactctttacgcagcgtgacccggcgtcgatcggcatgaacgagatcgccaaggccgcaggctgttcgcgcgcaacactgtatcggtacttcgacagccgcgaggcgctgcgaaccgcgtacgtgcaccgcgagacccgccggctcggccgcgagatcatggtgaagatcgccgatgtcgtcgaacctgccgaacggctgctggtgagcatcaccacgacgttgcggatggtccgcgacaaccccgcgttggccgcgtggtttaccaccacccgcccaccgatcggcggcgagatggccggacggtccgaggtgatcgcggccctggccgcggcattcctcaactcactaggtcccgacgatccgaccaccgtcgaacgccgcgcccgctgggtggtccggatgctcacatcgctgctgatgttccccggccgtgacgaagccgacgaacgagcgatgatcgcggagttcgtcgtcccgatcgtgacacctgcttctgccgccgctaggaaggccggtcaccctggacccgag

SEQ ID NO: 76 - DNA, 534 bp, Mycobacteria tuburculosis
atgcatccaatgataccagcggagtatatctccaacataatatatgaaggtccgggtgctgactcattgtctgccgccgccgagcaattgcgactaatgtataactcagctaacatgacggctaagtcgctcaccgacaggctcggcgagctgcaggagaactggaaaggtagttcgtcggacttgatggccgacgcggctgggcggtatctcgactggctgactaaacactctcgtcaaattctggaaaccgcctacgtgatcgacttcctcgcatacgtctatgaggagacacgtcacaaggtggtacccccggcgactatcgccaacaaccgcgaggaggtgcacaggctgatcgcgagcaacgtggccggggtaaacactccagcaatcgcaggactcgatgcacaatatcagcagtaccgggcccaaaatatcgctgtcatgaacgactatcaaagtaccgcccggtttatcctagcgtatctgccccgatggcaggagccgccgcagatctacgggggcgggggcggg

SEQ ID NO: 77 - DNA, 1230 bp, Mycobacteria tuburculosis
gtggccacgatagcccaacggctgcgtgacgaccacggggtggcggcgtcggagtcgtcggtgaggcgttggatcgcaacgcatttcgccgaggaggtggcccgcgagagagtcacggtgccgcgcggaccggtcgatgcgggtagtgaggcgcagatcgattacgggcggctgggcatgtggttcgacccggccaccgcgcgccgggtcgcggtgtgggcgttcgtgatggtgctggcgttctcccgacacctgttcgtgcgtccggtcatccggatggaccaaaccgcttggtgtgcttgccatgtcgccgcattcgaattcttcgacggggtgccggcgcggctagtgtgtgacaacctcaggaccggggtggacaagcccgacctgtacgacccgcagatcaaccgctcctacgccgagctggccagccactacgccacgctggtcgacccggcccgcgccagaaaacccaaagataaaccccgcgtggagcggccgatgacctatgtgcgggactcgttttggaaaggccgcgagttcgattcgctggcccagatgcagcaggcggcggtcacctggagcaccgaagtggccgggcttcggtacttacgtgccttggagggcgcccaacccctgcggatgttcgaagctgtggagcaacaagcgttgatcgcattgccgcccagggcatttgaactcaccagctggtcgatcggcaccgtcggggtggacacgcacctcaaagttggcaaggcactctattccgtgccgtggcggctgatcgggcaacgcctgcacgcgcgcaccgccggtgatgtggtgcagatcttcgccggcaacgatgtggtggccacccatgtgcgccgacccagcgggcgctccaccgacttctcccactacccaccggagaagatcgccttccacatgcgcaccccgacctggtgtcgacacaccgccgaactggtcggcccagccagccagcaagtgatcgccgaattcatgcgcgacaacgccatccaccacctacggtcggcccaaggcgtgctcgggctacgcgacaaacacggctgcgaccggctggaggccgcctgcgcccgcgccatcgaggtcggcgacccgagctatcgcaccatcaagggcatccttgttgccggcaccgaacacgccgccaacgagccgaccaccagtagtccggcaagcaccgctgggggcgttcctgcgcggccc

SEQ ID NO: 78 - DNA, 753 bp, Mycobacteria tuberulosis
atgtctatctgtgatccggcgctgcgtaatgcgctacgtaccctgaaactgtccggcatgctcgacaccctcgacgcccgcctggcccaaacccgcaacggcgacctggggcatctggaattcctgcaagcgttgcgtgaagacgagatcgcccgccgcgagtccgccgccctgacacgacgattacgccgcgccaagttcgaagcccaagccaccttcgaagacttcgacttcactgccaacccgaaactgcccggtgcgatgttgcgcgatctggccgcgctgcgctggctggatgccggcgaatcggtcatcctccacggcccggtcggcgtcggaaaaacccatgtagcacaagcacttgtccacgccgtggcccgccgcggcggcgacgtgcgcttcgccaaaacctcccgcatgctctccgacctcgccggcgggcacgccgaccgatcctggggccaacgcatccgcgaatacaccaagccgctcgtgctcattctggacgacttcgcgatgcgtgagcacaccgccatgcacgctgatgacctctacgagctcatcagcgaccgcgccatcactggcaaaccgctgatcttgaccagcaaccgcgcaccgaataactggtacggcctgttccccaaccccgtcgtcgccgaatcactcctggatcggctcatcaacaccagccaccaaatcctcatggacggacccagctaccgaccccgcaagagacccggccgcaccaccagc

SEQ ID NO: 79 - DNA, 696 bp, Mycobacteria tuberulosis
atgcatctaatgatacccgcggagtatatctccaacgtaatatatgaaggtccgcgtgctgactcattgtatgccgccgaccagcgattgcgacaattagctgactcagttagaacgactgccgagtcgctcaacaccacgctcgacgagctgcacgagaactggaaaggtagttcatcggaatggatggccgacgcggctttgcggtatctcgactggctgtctaaacactcccgtcagattttgcgaaccgcccgcgtgatcgaatccctcgtaatggcctatgaggagacacttctgagggtggtacccccggcgactatcgccaacaaccgcgaggaggtgcgcaggctgatcgcgagcaacgtggccgggggtaaacactccagcaatcgcagacctcgaggcacaatacgagcagtaccgggccgaaaatatccaagcaatggaccgctatctaagttggacccgatttgcgctatcgaagctgccccgatggcgggagccgccgcagatccacaggagcgggtaggtccaagaggccggcgcggtcttgcaggccagcaacaatgccgcggtcgaccaggcccatcgcttcgctgctcgcacgacacaccgcggtttcagatgaatcaggcgtttcacaccatggtgaacatgttgctgacgtgttttgcatgtcaggagaaaccgaga

SEQ ID NO: 80 - DNA, 528 bp, Mycobacteria tuberulosis
atgcatccaatgataccagcggagtatatctccaacataatatatgaaggcccgggcgctgactcattgtttttcgcctccgggcaattgcgagaattggcttactcagttgaaacgacggctgagtcgctcgaggacgagctcgacgagctggatgagaactggaaaggtagttcgtcggacttgttggccgacgcggttgagcggtatctccaatggctgtctaaacactccagtcagcttaagcatgccgcctgggtgatcaacggcctcgcgaacgcctataacgacacacgtcggaaggtggtacccccggaggagatcgccgccaaccgcgaggagaggcgcaggctgatcgcgagcaacgtggccggggtaaacactccagcaatcgcagacctcgatgcacaatacgaccagtaccgggcccgcaatgtcgctgtaatgaacgcctatgtaagttggacccgatctgcgctatcggatctgccccggtggcgggaaccgccgcagatctacaggggcggg

SEQ ID NO: 81 - DNA, 1170 bp, M. tuberculosis
atgatcatcgttgtcgggatcggcgccgacggcatgaccggtctctccgagcattctcgctccgaattgcgcagggccacagtaatttacggctcgaaacggcaacttgccctgctcgacgataccgtcaccgccgagcgctgggagtggccgacgccgatgctgcccgcggtgcaaggcctgtcaccggatggggctgacctacacgtggttgccagcggcgacccgttgttgcatggtatcggctccaccctgatccggctgttcggccacgacaacgtgaccgtgttgccgcacgtgtccgcggtgacgttggcgtgcgcccggatgggctggaacgtgtatgacaccgaggtgatcagcctggtcaccgcgcaaccacacaccgcggtgcgccgcggcggccgggccatcgtgctgtccggcgatcggtccaccccgcaggcgctggcggtgctgctgaccgagcacggtcgcggtgactccaagttcagcgtgctcgaacagcttggcggcccggccgaacgccgccgcgacggtacggcccgggcatgggcctgcgacccacccctcgatgtcgatgagctcaacgtgatcgccgtgcgctacctgctcgacgagcgcacgtcgtgggcacccgacgaggcattcgcgcacgacgggcagatcaccaaacacccgatccgcgtgctgaccctggctgcgctggcgccaaggcccgggcagcggttatgggacgtcggcgcgggctcaggcgccatcgcggtccagtggtgtcggagctggccgggctgcaccgcggtggcgttcgagcgcgacgaacggcgccgccgcaacattgggttcaatgccgcggccttcggggtgagcgtcgacgtgcgcggcgacgcgcccgatgcgttcgacgacgccgcacggccgtcggtgatttttcttggcggtggtgtaacccagccaggcctgcttgaggcctgcctggacagcctgcccgcaggcgggaacttggtcgccaacgctgtcaccgtcgaatcggaagccgctctggcgcatgcatattcgcgcctcggtggcgagctacgacgattccagcactatctcggcgaaccgctgggcggcttcaccggttggcgcccacagctgccggtcacccagtggtcggtgaccaagcga

SEQ ID NO: 82 - DNA, 747 bp, Mycobacteria tuberulosis
gtggacgacacgggcgctgctccggtagtaattttcggcggccgcagccagatcggcggcgaactcgcgcgacgcctggctgccggggcgacgatggtgctggccgcgcggaacgccgatcaactcgccgaccaggccgccgcactccgcgcagctggcgctatagcggtgcacacccgggagttcgacgccgacgacctggccgcacacggcccgttggtcgcttcgctcgttgccgagcacggccccatcggcaccgcggtgctggccttcgggatactcggcgaccaggcccgcgccgagacagacgcggcgcacgcggtggccatcgtgcacaccgactacgtcgcccaggtcagcctgctgactcatctggcagcggcgatgcgcaccgccggacggggatcgctggtggtgttctcctcggtcgccgggattcgggtgcgccgcgccaactatgtctacggatcggccaaagccggcctggacggcttcgccagcggcctggccgatgcgttgcacggcaccggggtgcggttactgatcgcgcggccgggattcgtcatcgggcgcatgaccgagggcatgacgcccgcacccctgtcggtcaccccggagcgggtggccgccgcgaccgcgcgtgcgctggtcaacggtaagcgcgtggtgtggattccgtgggcgctgcggccaatgtttgttgcgctgcggttgcttccccggttcgtctggcgcaggatgccgcga

SEQ ID NO: 83 - DNA, 411 bp, Mycobacteria tuberulosis
gtggcgatggtcaacaccactacgcggcttagtgacgacgcgctggcgtttctttccgaacgccatctggccatgctgaccacgctgcgggcggacaactcgccgcacgtggtggcggtaggtttcaccttcgaccccaagactcacatcgcgcgggtcatcaccaccggcggctcccaaaaggccgtcaatgccgaccgcagtgggcttgccgtgctcagccaggtcgacggcgcgcgctggctctcactggagggtagggcggcggtgaacagcgacatcgacgccgtgcgcgacgccgagctgcgctacgcgcagcgctatcgcaccccgcgtcccaatccacgccgagtggtcatcgaggtccagattgagcgcgtgctgggatccgcggatctgctcgaccgggcc

SEQ ID NO: 84 - DNA, 1461 bp, Mycobacteria tuberulosis
atgccccgcgcccgatggctgcagagcgcggccctcatgggcgccttggccgtggtgttgataaccgcggcaccggtggccgccgatgcctaccaggtgcccgctccgccctcgcccaccgcatcctgtgacgtaataagcccggttgcgatcccctgcgtggcgctcggcaagttcgccgacgcggtcgctgcggagtgtcgccgcgtcggtgtgcccgatgcgcggtgcgtgcttccgctcgcgcaccgggtgacccaggccgcgcgtgatgcctacctacagtcttgggtgcatcgcaccgcgcggttccaggatgcgttgcaagacccggtgccgctgcgggaaactcagtggctcggcacgcacaactcgttcaacagcctcagcgattcgttcacggtctcgcacgcagactcaaaccagcagctgtcgttggcccaacagctcgacatcgacgtccgcgcgctcgagctagacctgcactacttgccccgcctcgagggccacggcgcccccggcgtcaccgtgtgtcacgggctgggaccgaagaacgcgaacctaggctgcaccgtcgaacctctgctggccacagtgctgccgcagatcgccaactggttgaacgcacccgggcataccgaggaggtcatcctgctctacctggaggaccagctgaagaacgcgtcggcgtatgagtcggtggtggctaccctcgaccaagtgttgcggcgtgcggacggaacaagccttatctaccgtcccaacccggcccggcgtgccaccaacggctgtgtcccgcttccactcgacgtgtcgcgggaggaaatccgcgcatccggcgcacgagccgtgctcgtcgggtcttgtgcgccaggttggtcggccgccgtcttcgactggagcggcgttgagctggaaagcggctcgaactccggctaccggccatacccggcctgcgatgccacctatggccgcggtgtctacgcttggcgactggtccgctattacgaggactccacgctggccacggcgttggccaacccgacccgtccaccggccaatccgcaggcgcttaccccgccgaaggtgccggcgatgaccgattgcggggtcaatctgttcggcttcgatcagctgctccccgaagacggccgcattcaggcgtcgttgtggagctgggcaccggacgaaccgcgtgccggtgccggagcatgcgccctgcagggcgcggatggccgctgggtcgccgcatcgtgcggtgacccacaccctgcggcctgtcgggacgcggcaggcaggtggaccgtgacgccggcacccgtggtcttcgccggggctgccctagcctgcacagccatcggcgcggactttaccctgccccgaacgggcaatcagaacgcccgtctgcacgccgtggccgggcccgccggtggcgcctgggtgcattacctactgccgcca

SEQ ID NO: 85 - DNA, 429 bp, Mycobacteria tuberulosis
atgaccaccacgccccgacaacccctgttctgcgcccacgccgacaccaacggcgacccgggccgctgcgcctgcggccagcagctcgccgacgtcggcccggccaccccgccaccgccctggtgcgaaccgggcaccgaacccatctgggagcagctcaccgaacgatacggcggcgtcacaatctgccagtggacacgatattttccggccggcgacccggtggctgccgacgtgtggatcgccgccgacgatcgtgtcgttgacggccgggtgctgcgcacccaaccggcgattcactacacggaaccgcccgtgttggggatcggcccggcggcggcccgccggctggccgctgagctgctcaacgccgccgacaccctcgacgacggccgccggcagctagacgacctcggcgaacaccggcgg

SEQ ID NO: 86 - DNA, 996 bp, Mycobacteria tuberulosis
gtgaacaccgcgacccgggtccggctggcccgcaaacgcgccgaccggctcaatctgaaactaatcaagaacggccaccacttcaggttgcgtgacgccgacgagatcacgctggcggtcgggcacctaggggtggtggaagccttcctggcggcggccaagtcgcaaaacaagccgcccggtccgccgccgagcctccacgccccgccatcctggcggcgcgacatcgacgactacctgctcaacctgaacgccgccggtcaacgcccagcgacgatccggctacgcaagacggtgctgtgcgcagccgcccacggcctcggccgcccacccgccgacgtcaccgccgaacacctcctggactggctaggcaaacagcagcacctctccccagagggccgcaaaacctatcgcagcacgttgcggggcttcttcgtgtgggcctacgaaatggaccgggtgcgcgactatgtcgcagactccctgcctaaggtgcgctgcccgaaacagccgccccgcccggccggcgacgacgtctggcaagcggcgctggccaaggccgaccgtcgaatcgagctgatgatccgcctagccggtgaggccgggctgcgacgcgccgaagccgcccaggcgcacaccggcgacttgatggacggcgggcttctcctcgttcacggcaaaggtggtaaacgccgtattgtgccgatcagcgactacttggccgcgctcatccgcgacaccccgcacggctacctgttccccaacggcaccggcggccacctcaccgccgaacacgtgggaaaactcgtctcccgggcattacccggtgacgcgaccatgcacaccctgcggcaccgatacgccacccgcgcctaccgcggctcccacaacttgcgagctgtacaacaacttctcggtcacgcctcgatcgtgacaacagaacgctacacagcgctgtgcgacgacgaggtgcgcgccgcagcagcagccgcatgg

SEQ ID NO: 87 - DNA, 366 bp, Mycobacteria tuberulosis
gtgcacgtgtgccacacgatcgccgacgtggtcgaccgggccaaagccgaacgctccgaaaacacgcttcgcaaggatttcaccccctcggagctgctcgccgctggtcgccggatcgccgagctggaacggccgaaagccaaacagcggcaacgcgaaggcggcgaccatggccgccaggctcgatattctggcttaggctccatggagcctaagccagaatcagagcgcgatgcccacaaagccgacactgccatcagcgaagccctcggcatctcccgcggccactaccagcggctcaaacgaatcgacaacgcaacccgcagcgaagctggctaccgggatggtttaaacggttggagcggc

SEQ ID NO: 88 - DNA, 324 bp, Mycobacteria tuberulosis
atgtcaggtggttcatcgaggaggtacccgccggagctgcgtgagcgggcggtgcggatggtcgcagagatccgcggtcagcacgattcggagtgggcagcgatcagtgaggtcgcccgtctacttggtgttggctgcgcggagacggtgcgtaagtgggtgcgccaggcgcaggtcgatgccggcgcacggcccgggaccacgaccgaagaatccgctgagctgaagcgcttgcggcgggacaacgccgaattgcgaagggcgaacgcgattttaaagaccgcgtcggctttcttcgcggccgagctcgaccggccagcacgc

SEQ ID NO: 89 - DNA, 984 bp, Mycobacteria tuberulosis
aaagaccgcgtcggctttcttcgcggccgagctcgaccggccagcacgctaattacccggttcatcgccgatcatcagggccaccgcgagggccccgatggtttgcggtggggtgtcgagtcgatctgcacacagctgaccgagctgggtgtgccgatcgccccatcgacctactacgaccacatcaaccgggagcccagccgccgcgagctgcgcgatggcgaactcaaggagcacatcagccgcgtccacgccgccaactacggtgtttacggtgcccgcaaagtgtggctaaccctgaaccgtgagggcatcgaggtggccagatgcaccgtcgaacggctgatgaccaaactcggcctgtccgggaccacccgcggcaaagcccgcaggaccacgatcgctgatccggccacagcccgtcccgccgatctcgtccagcgccgcttcggaccaccagcacctaaccggctgtgggtagcagacctcacctatgtgtcgacctgggcagggttcgcctacgtggcctttgtcaccgacgcctacgctcgcaggatcctgggctggcgggtcgcttccacgatggccacctccatggtcctcgacgcgatcgagcaagccatctggacccgccaacaagaaggcgtactcgacctgaaagacgttatccaccatacggataggggatctcagtacacatcgatccggttcagcgagcggctcgccgaggcaggcatccaaccgtcggtcggagcggtcggaagctcctatgacaatgcactagccgagacgatcaacggcctatacaagaccgagctgatcaaacccggcaagccctggcggtccatcgaggatgtcgagttggccaccgcgcgctgggtcgactggttcaaccatcgccgcctctaccagtactgcggcgacgtcccgccggtcgaactcgaggctgcctactacgctcaacgccagagaccagccgccggc

SEQ ID NO: 90 - DNA, 1437 bp, Mycobacteria tuberulosis
atgactaatgaacaacatttcgctgacgatggcgacatcaaacagctcagcctcgacgaaacccgttccgcggcaaaacagctcctcgactccgtcgagggcgacctgaccggtgatgtggcgcaacgttttcaggcgctgacacgccacgccgaggaactgcgggcggagcagcgccgccgcggccgcgaagccgaggaggcgctgcgccgctgccgggccggtgagctgagggtggtgcccggtgctcccaccggcggcgacgacggcgacgcgccgccgggcaactcgttgcgcgacatcgcgtttcgcacactggacgtttgtgtgcgcgatggcctgatgtcgtcgcgggcggcggaagccgcggaaaccttgtgccgcaccgggccgccgcagtcgacgtcgtgggcgcagcgctggctggcggccaccggcaaccgcgactacctgggggcgttcgtcaagagggtttcgaaccctgttgcggggcacacgacctggaccgaccgggaagcggccgcgtggcgtgaggcggccgcggtggccgccgagcagcgagcaatgggcttggtggacaccgccggcgggtttttgatcccggcggcgctggatccggcgattctgctgtcgggtgatggttcaacgaatccgatccggcaggtggcgagggtggtgcaaacgacctccgaggtttggcggggcgtgacctccgaaggcgccgaggctcattggtactccgaagcccaggaggtgtccgacgattcgccaacgctggcccagccggcggtgccgagctaccgtggctcctgctggattccgttcagtctcgagattgagggtgacgccgccggattcgtcgcagaggtgggccgcgtcctagcggattcggttgagcagctgcaggcggcggcgttcgtcagcggctccggcaacggcgagcccaccggattcgtctccgcactgaccggcaccgcggactacaccgtcaccggcgcggggacggaagccgttgtagccgccgacgtttacgcgctgcagtcggcgttgccgccgcgctttcaatccaacagcgcgttcgcggcgaacttgtccaccatcaacgtgctgcgccaggcggaaaccgcgaatggggcgctgaaattcccatcgctgcacgccagcccgccgatgctggccgggaaacacatctgggaggtgtcgaacatggacaccgtggacgcggcggtgaccgccaccaattacccgctggtgcttggcgactggaagcagttcatcatcaccgaccgggtcgggtcgacggtggagctggtgccgcacgtgttcggcggcaaccgccgaccgaccggacagcgcggattcttctgctggttccgagtcggttctgatgtgctggtggacaatgcgttccgcgtgctgaaggtgcagaccaccgcg

SEQ ID NO: 91 - DNA, 531 bp, Mycobacteria tuberulosis
ttgagtagcatccttttccgcacggccgagctgcggcctggtgagggccgcaccgtgtacggcgtcatcgtgccttatggcgaggtgaccaccgtccgcgacctcgacggcgagttccgggaaatgttcgctcctggcgcttttcggcgctccatcgctgagcgcggccacaaggtgaagctgctggtctcccacgacgctcgaacccgctacccggttggccgggccgtcgagctgcgtgaggagcctcacggcttgttcggggcgttcgagcttgcgaacaccccggacggcgacgaggccctggcgaatgtgaaagctggtgtggtggacgcgttttcggtgggtttccggccgatccgggaccgccgggaaggggatgtgatcgtgcgggtcgaggcggcgctgttggaggtctccttgaccggcgttccggcctatctgggcgcgcagatcgccggtgtgcgcgcggaatcgcttgcagtcgtttcccgttcgctagccgaagccaggttagccctgatggattgg

SEQ ID NO: 92 - DNA, 624 bp, Mycobacteria tuberculosis
ttgccatcgccagcaaccgcccgaccggacaccgccacggtgggagagcgtgtgcgcgctcaagttttatggggcgttttttggcatcatggcattcgcgacccgaaacccggaaagaggagggtggtgttgaaaatgggtaggcgtggtcccgcgccggcgccggcgcagttgaaactcctcggcggccgctcgccgggccgtgattctggcggccggcgggttacaccaccggcggcgttcgagcgtgttgcgccggaatgcccggattggttgccgccaggcgctaaagacatgtgggggcgcgtcgttcccgagcttgcggcattaaacctgctgaaggagtccgaccttggggtgctgacctccttctgcgtcgcctgggatcagctcatgcaggctgtaacagcctaccgtgaacagggtttcatcgcgacgaacgcccgcagccgacgggtgacggtgcatcctgccgtggccgcggcccgggccgcgacgagggacgttttggtgctcgcgcgcgaattggggtgcacgccaagcgctgaggcgaatttggctgctgtgctggcggcggcgggggaccccgacgacgacgagttcaacccgttcgccccagaccgg

SEQ ID NO: 93 - DNA, 321 bp, Mycobacteria tuberculosis
ttgacccacaagcgcactaaacgccagccagccatcgccgcagggctcaacgccccgcgtcggaatcgcgttgggcggcaacatggttggccggccgacgttccgtccgccgagcagcgccgcgcccaacggcagcgcgacctcgaggctatccgccgagcgtacgccgagatggtggcgacatcacacgaaatcgacgacgacacagccgaactggcgctgttgtcgatgcatctcgacgatgagcagcgccggcttgaggcggggatgaagctcggctggcatccgtatcacttccccgacgaacccgacagcaaacag

SEQ ID NO: 94 - DNA, 243 bp, Mycobacteria tuberculosis
atgagcggccacgcgttggctgctcggacgttgctggccgccgcggacgagcttgtcggcggcccgccagtcgaggcttcggccgccgcgctggccggcgacgccgcgggcgcatggcggaccgcggccgtcgagcttgcgcgagcgttggtccgcgctgtggcggagtcgcacggcgtcgcggccgttttgttcgccgcgacggccgccgcggcggcggccgtcgaccggggtgatccgccg

SEQ ID NO: 95 - DNA, 1425 bp, Mycobacteria tuberculosis
atggctgacatcccctacggccgtgactatcccgacccgatctggtgtgacgaggacggccagccgatgccgccggtcggcgccgaattgctcgacgacattagggcattcttgcggcggttcgtagtctatccaagcgaccatgaactgatcgcgcacaccctctggattgcgcattgctggtttatggaggcgtgggactcaacgccccgaatcgcttttttgtcaccggaacccggctctggcaagagccgcgcactcgaagtcacggaaccgctagtgccccggccggtgcatgccatcaactgcacaccggcctacctgttccgtcgggtggccgatccggtcgggcggccgaccgtcctgtacgacgagtgtgacaccctgtttggcccgaaagctaaagaacacgaggaaattcgcggcgtgatcaacgccggccaccgcaagggagccgtcgcgggccgctgcgtcatccgcggcaagatcgttgagaccgaggaactgccagcgtactgtgcggtcgccttggccggcctcgacgacctgcccgacaccatcatgtctcggtcgatcgtggtgaggatgcgcaggagggcaccaaccgaacccgtggagccgtggcgcccccgcgtcaacggccccgaggccgagaagctgcacgaccggttggcgaactgggcggccgccattaacccgctggaaagcggttggccggcgatgccggacggggtgaccgaccggcgcgccgacgtctgggagtccctggttgcggttgctgacaccgcgggcgggcactggcccaaaaccgcccgtgcaaccgcagaaacggatgcaaccgcaaatcgaggagccaagcccagcataggcgtgctgctgctgcgggatatccgtcgagtcttcagcgaccgggaccggatgcgcaccagcgacatcctgaccggactgaaccggatggaggagggaccgtggggctccatccgccgcggcgacccgctcgacgcgcgcggcctcgcgacccggctcggcagatacggcatcgggccgaagttccagcacagtggtggcgaaccaccctacaaagggtattcgcggacccagttcgaggatgcgtggtcccggtatctctctgccgacgacgaaacccccgaggaacgagatttatcggtttccgcggtttccgcggtttcaccgccggttggcgatcccggtgatgcaaccggcgcaaccgatgcaaccgatctcccggaggcgggcgacttgccgtacgagccgccggcgcccaacgggcaccccaacggcgacgcgccgctgtgctccgggccgggatgccccaacaagctcctcagtactgaggccaaggccgccggcaaatgccggccctgccgaggtcgagcggcggctagcgctcgggacggcgcccga

SEQ ID NO: 96 - DNA, 390 bp, Mycobacteria tuberculosis
atgaccgccgtcggcgggtcgccgccgacgcgacgatgcccggccacagaggaccgggcacccgcgacagtcgccacaccgtctagcaccgatcctaccgcgtcccgcgccgtgtcgtggtggtcggtgcacgagtatgtcgcaccgaccctggccgccgccgtggaatggccgatggccggcaccccggcgtggtgcgacctcgacgacaccgacccggtcaaatgggccgcgatctgcgacgctgctcggcattgggcactccgggtggagacgtgccaggccgcgtcggccgaggcatcacgtgacgtatccgccgccgccgactggccggcggtctctcgggagatccagcgtcggcgtgacgcctacattcggcgggtggtggtc

SEQ ID NO: 97 - DNA, 258 bp, Mycobacteria tuberculosis
atgtgcgcgttcccgtcgccgagtctcgggtggacggtctctcacgagaccgaaaggcccggcatggcagacgctcccccgttgtcacggcggtacatcacgatcagtgaggccgccgaatatctagcggtcaccgaccgcacggtccgccagatgatcgccgacggccgcctacgcggataccgctccggcacccgcctcgtccgtctgcgccgcgatgaggtcgacggcgccatgcacccgttcggtggtgccgca

SEQ ID NO: 98 - DNA, 360 bp, Mycobacteria tuberculosis
atggccgatgcggttaagtacgtagttatgtgcaactgcgacgacgaaccgggagcgctcatcatcgcctggatcgacgacgaacgacccgccggcgggcacatacagatgcggtcgaacacccgcttcaccgaaacacagtggggccgccatatcgagtggaaactcgaatgccgggcatgccgaaagtatgcgccgatatccgagatgaccgccgcggcgatcctcgacggtttcggggcgaagcttcacgagctgagaacgtcgaccatccccgacgctgacgatccatcaatagcagaggcgcgacacgtaattccgttcagcgcattatgcttgcgcttgagccagctaggcggg

SEQ ID NO: 99 - DNA, 1125 bp, Mycobacteria tuberculosis
gtgacgcaaaccggcaagcgtcagagacgcaaattcggtcgcatccgacagttcaactccggccgctggcaagccagctacaccggccccgacggccgcgtgtacatcgcccccaaaaccttcaacgccaagatcgacgccgaagcatggctcaccgaccgccgccgcgaaatcgaccgacaactatggtccccggcatcgggtcaggaagaccgccccggagccccattcggtgagtacgccgaaggatggctgaagcagcgtggaatcaaggaccgcacccgcgcccactatcgcaaactgctggacaaccacatcctggccaccttcgctgacaccgacctacgcgacatcaccccggccgccgtgcgccgctggtacgccaccaccgccgtgggcacaccgaccatgcgggcacactcctacagcttgctgcgcgcaatcatgcagaccgccttggccgacgacctgatcgactccaacccctgccgcatctcaggcgcgtccaccgcccgccgcgtccacaagatcaggcccgccaccctcgacgagctggaaaccatcaccaaagccatgcccgacccctaccaggcgttcgtgctgatggcggcatggctggccatgcgctacggcgagctgaccgaattacgccgcaaagacatcgacctgcacggcgaggttgcgcgggtgcggcgggctgtcgttcgggtgggcgaaggcttcaaggtgacgacaccgaaaagcgatgcgggagtgcgcgacataagtatcccgccacatctgatacccgccatcgaagaccaccttcacaaacacgtcaaccccggccgggagtccctgctgttcccatcggtcaacgaccccaaccgtcacctagcaccctcggcgctgtaccgcatgttctacaaggcccgaaaagccgccggccgaccagacttacgggtgcacgaccttcgacactccggcgccgtgttggctgcatccaccggcgccacactggccgaactgatgcagcggctaggacacagcacagccggcgccgcactccgctaccagcacgccgccaagggccgggaccgcgaaatcgccgcactgttaagcaaactggccgagaaccaggagatg

SEQ ID NO: 100 - DNA, 225 bp, Mycobacteria tuberculosis
gtgatagcgggcgtcgaccaggcgcttgcagcaacaggccaggctagccagcgggcggcaggcgcatctggtggggtcaccgtcggtgtcggcgtgggcacggaacagaggaacctttcggtggttgcaccgagtcagttcacatttagttcacgcagcccagattttgtggatgaaaccgcaggtcaatcgtggtgcgcgatactgggattgaaccagtttcac

SEQ ID NO: 101 - DNA, 186 bp, Mycobacteria tuberculosis
atgatcgagcagggccgcgactgccgggacgtggtcacccagctcgccgcggtatcgcgcgcactcgaccgcgccggattcaagatcgttgcggcagggttgaaggaatgcgtgtccggggccacggccagcggcgcggcaccgctgagtgcagctgagctagaaaagctgttcctggcgctcgct

SEQ ID NO: 102 - DNA, 357 bp, Mycobacteria tuberculosis
atgtcggaccagccacgtcatcaccaggtcctcgacgacctgctgccccaacaccgcgctctacgtcaccagattccccaggtgtaccagcgatttgtagccctgggcgacgccgcgcttaccgacggcgctctcagccgcaaggtcaaggagcttgtggcgctggcgatcgcggttgtgcaggggtgcgatggctgcgtcgcatcacacgcccaagccgcggtacgggccggcgctacagcgcaagaagccgctgaggccatcggggtcaccatcttgatgcacggtggaccggccaccatccacggtgctcgtgcctacgcggcattttgcgaattcgctgacacaacgccgtcc

SEQ ID NO: 103 - DNA, 1854 bp, Mycobacteria tuberculosis
atgtcctatctcgtcgtggtgccggagttggtcgcagcggcggcaacagatttggcgaacatcggttcgtcgattagtgcagccaacgcggccgcggcggcaccgaccacggcactggtcgcagccggcggcgacgaggtatcggcggccatagccgcgttgttcggagcgcatgctcgggcatatcaagcgttgagtgcccaggcggcgatgtttcatgaacagtttgtccgggccctcgccgccggcggtaactcctacgccgtcgctgaggcggcaaccgcgcaatcggttcagcaagatctgctcaacctgatcaatgcgcccacccaggcgctgttggggcgtccgctgatcggcaacggcgccaacgggctgccgggtacgggccagaacggcggcgacggcgggattctgtacggcaacggcggcaacggtgggtccggcggggtcaaccaggccggtggcaatggcgggaatgctgggctgtggggcaatggcggatccggcggagccggcgggaacgccaccactgccggccgcaacggcttcaacgggggcgccgggggaagcggcggtttgctgtggggcaatggcggtgccggcggggccggtgggaacggcggtccggctccgctcgtgggcggggtgggcaccaccggtggcgccggcgggaacggcggcggcgccgggttgttctacggtttcggcggcgccggtgggaacggcgggatgggcggggtggcaccgagcaccggcccctcgatgggcatcctcccggccggcggtgtcggcgggcctggtggctccggcggggcgagcgcgcttgccttcggctccggcggcgtcggcggtgccggtggcttgggcgggccgaccgatggcaccgtccagggggtgggcggcttcggcggtcagggcggcaacggcgggcagagcggcttgttgtttggcaacgcgggagccggcggggcaggcgctgccggcggagccggcaccggcgacaccgagagcttcggcggccacggcggggccggcggtgatggcggcgctgttggcttgatcggtaacggcggggccggcggcaccggatctcccggcgctgtggtgggtggtaacggcggcgtcggtggtctgggtggcgccggcagtcccgggggtctgttgtacggcaccgggggggccggcggcaatggcggaccgggtggtgacggtggtactggcgcgacggtgggctttgccggctccggcggtttcggcggtgcggggggcatcgcccagctgtttggcacgggtggcatgggtggtagcggcggtggtataggcgctggcaccacgaccgtggtgccgcccgacgtcgccccggtgggtggcacaggcggcaatggcggtcgcgccgggctgctgttgggtgtgggtggcatgggcggtaatggcggtgccaccagcgtcggcgggacgctctacgccgccggtggaaacggcggcgacggcgggttggtgtggggcaacggtggcaccggcgggagcggtggcgccggcggggcgggcagcgtcggcaacggcggtgcgggtggcaacgcggcactgctgttcggcaacggcggggcgggcggggccggcggcgccggcggcatcggtgccggcggagccggcggcttcggcgcggttctgtttggcaacggcggggctggcgggagcggtgcccccggtggcatcggcgccggtggcaatggcggaaacgcgctgctggtcggcaacggcggcaacggtggggcaggtaccggtggggctgctggcggtgccggtggctcgggcgggttgctattcggccaaaatgggatgcccgggccg

SEQ ID NO: 104 - DNA, 1242 bp, Mycobacteria tuberculosis
gtgcatgaggtggctgctcgtgagcaacgttcggacgggccgatgaggctggatgcgcagggccgactgcagcgttacgaggaggcgttcgctgactacgatgcaccgtttgcgttcgtagatctcgacgcgatgtggggcaatgccgatcaactgcttgcgcgcgccggcgacaagccgatccgggtggcgtcgaagtcgctgcgttgccgaccactgcaacgcgaaatccttgatgccagtgagcgattcgacgggctattgacgttcacgcttaccgagacgctgtggcttgccggccaaggtttctcgaacctgttgttggcctacccgccgaccgaccgggcggcattgcgtgcgcttggcgagctgacggccaaggacccggacggggcgccgatcgtgatggtggacagcgtggagcaccttgacctgatcgagcgcacgaccgacaagccggtacggctgtgtctggatttcgatgccggctattggcgcgccggcgggcggataaaaattggttccaagcgctcgccgctgcacaccccggagcaggctcgcgcactcgcggtggagatcgcgcggcggccggcgctaacgttggcggcgttgatgtgctacgaggcccacattgcgggcctcggtgacaacgtcgccggcaagcgggtccacaacgcgatcatccgtcggatgcagcgcatgtcgttcgaagagctgcgcgagcgtcgtgcccgggccgtcgagctggtgcgcgaggtcgccgacatcaagatcgtcaacgccggtggcaccggcgacttgcagctggttgcgcaggagccgttgattaccgaagcgaccgccggctcgggtttttacgcgccgacactgttcgactcgtattcgacgttcacgctgcagcccgcggcgatgttcgcgctgccggtatgccgtcgtcccggtgcaaagaccgtgaccgcgctcgggggtggctatttagccagcggggtcggggcgaaggaccgcatgccgactccctacctgccggtcgggctgaagctcaatgcgctggagggaacgggcgaagttcagacaccgctatccggtgatgcagcccgacggctgaagcttggcgacaaggtctacttccgccacaccaaggccggtgagctgtgtgagcggttcgaccatctgcatctggtccgtggcgctgaagtagtcgacaccgtccccacctaccggggtgaagggcgcaccttcctc

SEQ ID NO: 105 - DNA, 1284 bp, Mycobacteria tuberculosis
atggacgaggcccacccggctcacccggcagatgcggggcggcccggtggcccaattcaaggcgcgcgaagaggagctgccatgacaccgatcaccgccctgccgaccgagttggcggccatgcgcgaggtagtcgagacgctcgcacccattgagcgtgccgcgggcgagccgggtgagcacaaggcggccgagtggatcgtcgagcgcctgcgcacggcgggcgcgcaggacgcgcgcatcgaggaggagcagtacctcgacggctacccgaggctgcacctcaagctgtcggtgatcggggtggcggccggcgtcgcgggcctgctcagcagacgtttgcgcatccccgccgcgctggccggggtgggtgcggggctggcaatcgccgacgattgcgccaacgggccgcgcattgtgcgcaaacgaacggagacgccccggacgacatggaacgcggtagccgaggccggtgatcctgctggtcagctaacagttgttgtgtgcgctcaccacgacgccgcgcacagcggcaagtttttcgaggctcatattgaggaggtaatggtcgagctgtttcccgggattgtggagcgcatcgacacgcagctgccgaactggtgggggccgatcctcgcgcccgcactcgccggtgtcggcgccctgcgcggcagccggccgatgatgatcgccggaacggtgggtagcgccctggccgccgctttgttcgccgacatcgcgcgcagtccggtcgtccccggtgccaacgacaatctctccgcggttgcgctgctggtcgcgctggccgagcggctgcgcgagcggccggtgaagggcgtgcgagtgttgctcgtgtccctgggggccgaggaaacgttgcagggcgggatctacgggttcctggcgcgacacaaacccgagctggaccgcgaccgcacatacttcctgaacttcgacaccatcggctcacccgagctcatcatgctcgagggcgagggcccgacggtcatggaggactacttctatcggccattccgggatctggtcatccgggcggccgagcgcgccgacgcgccgctgcggcgcggcatccggtcgcgcaacagtaccgacgcggtgttgatgagccgcgccggctacccgaccgcgtgctttgtgtcgatcaaccggcacaagtcggtggccaattaccacctgatgtccgatacacctgagaatctctgctatgagacggtgtcccacgccgtcaccgtcgccgaatccgtgatcagggagctggcccga

SEQ ID NO: 106 - DNA, 1284 bp, Mycobacteria tuberculosis
atgagcccgatatggagtaattggcctggtgagcaagtctgcgcgccgtcggcgatcgtacggccgacctcggaggctgagctggccgacgtgatcgcgcaggcggcgaaaagaggcgagcgggtacgcgcggttggcagcgggcattcgtttaccgacatcgcctgcacggacggggtcatgatcgacatgaccggcctgcagcgggtcctcgacgtggaccagccgactggcctggtgacggtcgaggggggcgcaaagctacgtgcgctgggaccccaattggcgcaacgacggctcggcctggagaaccagggtgacgtggatccccaatccatcaccggcgcgaccgcgaccgcgacgcacggaaccggggtgcgtttccagaatctgtcggcgcggatcgtttcgctgcggctggtcaccgcgggcggggaagtgctcagtctgtccgaaggtgacgattacctggcggcacgggtttccctcggcgcgctaggagtgatctcacaggtcaccctgcagacggttccgctattcacgttgcatcgccatgatcagcgacgctcgctggcgcagacgctggagcgcctcgacgagttcgtggacggtaatgaccatttcgagtttttcgtattcccttacgcagataaggcgttgacgcgcaccatgcatcgcagtgacgagcagcccaaacccacgcccgggtggcagcgcatggtcggcgagaacttcgagaacgggggattgagcctgatctgccagaccggccgtcgttttcctagtgtggcgccgcgactgaaccgcctgatgacgaacatgatgtcgtcctccaccgtgcaagaccgcgcctacaaggtctttgcgacccaacgcaaggtcaggttcaccgagatggagtacgcgatcccgcgtgaaaacgggcgcgaggcgctccagcgtgtcatcgaccttgtgcgccgtcgcagcttgccgatcatgtttccgattgaggtgcgattctccgcccccgacgattccttcctgtcgaccgcatatgggcgcgacacttgctacatcgcggttcatcaatacgccggtatggagttcgaaagctacttccgcgccgtcgaggagatcatggacgactacgccggtcggccacactggggtaaacgtcactatcagaccgccgccacgcttcgtgagcgctatccgcagtgggatcggttcgccgcggttcgcgatcgcctcgatccggaccgggtgtttctcaacgactacacccggcgcgttctcggtccc

SEQ ID NO: 107 - DNA, 309 bp, Mycobacteria tuberculosis
ttgggttcaacaggaggtagccaacccatgacggcgaatcgagggcccgctgcaatctcgagcggctcgaactctggccgcgttctcgacaccgcccggggtatcctcatcgctcttcggcggtgccccgcagagaccgcgttcgacgagttgcacaacgccgctcaacggcacagattgccggtcttcgaaatagcttgggcactagtgcatttggcggtcgagggaagcacgccatgccggagcttcgtcgatgcccagtcggcggctcggcgggagtggggtcagctttttgcgcatgcggcggcg

SEQ ID NO: 108 - DNA, 744 bp, Mycobacteria tuberculosis
gtgccgcctacggaaggaaagtcgacaacgaatcgcgacgaaggcatccaggtgctccgtcgcgccgtcgccgcgctggacgaaatagctgccgaaccgggacacctgcgcctagtcgatctctgcgagcggctggggctggccaaatcgacgactcgacgcttgctggtcggcctggtcgaggtggggctggttagtgtcgattcgcacggccgcttcgcactgggcgagcgtttgctgggattcggaagtgtcaccggagcccacatagccgcggcgttccggccgaccgtcgagcgagttgcccgcgcgaccgacggcgaaacggtcgacctgtcggtactgcgcggccagcgaatgtggtttgtcgaccagatcgaatcgtcttaccggctgcgtgcggtctcagccgtcgggctccgcttcccgttgaacggaaccgcgaatggaaaagcggcgctggctgctctcgacgacgccgacgccgaggccgcgctctgccgtctggatcccatggtggccgaaggtctacggcgcgagatcgtcgagatccggcgcaccggtatcgctttcgaccgcaacgagcacaccccagggatatccgcggctgcgatcgcacgacgcgccctgggcgacaacgtgatcgcgatctcggtgccggcgcccaccgcacgatttctggaaaaagagcagcgcataatcgccgcgttgcgcgccgccgcggactcgccggactggactcgc

SEQ ID NO: 109 - DNA, 1218 bp, Mycobacteria tuberculosis
atggcatccgtcgcccaacccgttaggcgccgcccaaaggaccggaagaagcagattttggatcaggccgttggactgttcatcgaacgtggcttccattcggtcaaattggaggacattgccgaggcggccggggtgaccgcgcgcgcgttgtatcgccactacgacaacaagcaggcgttgctcgccgaagcgatccgaaccggccaggatcagtaccagagcgcgcgtcgtctcaccgagggcgagacggagccgacgccgcggccgttgaacgccgatctggaagacctgatcgccgcggcggtcgcctctcgggcgttgacggtgctgtggcagcgcgaggcccgctacctcaacgaggacgaccgcacggcggtccggcgccgcatcaacgcgatcgtcgccggcatgcgtgacagcgtgctgctggaggtgcccgatctgagtccacagcattcggagttgcgggcgtgggcggtgtccagcactttgaccagcctgggccggcacagcctaagcctgccgggcgaggaactgaaaaagcttctctaccaggcgtgtatggccgcggcaaggacgcctcccgtctgcgaattgccgccactgccggccggtgatgccgcacgcgacgaggccgacgtgctgttctcccgctacgagaccctgctggccgcgggcgcgcggctgttccgtgcgcagggctatccggccgtcaacaccagcgaaatcggcaagggagccggcatcgcgggcccggggctgtaccgttcgttttcttccaaacaggccatcctggacgcgctcatccgccgcctcgacgagtggcgctgcctggagtgcatccgagcgctacgagcgaatcagcaagcggcacaacggttgcgcggccttgtccaagggcacgttcggatcagcttggacgctccggatctggtggcagtgtcggtcaccgaactgtcgcacgcctctgtcgaagtacgcgacggctacctgcgaaatcagggcgaccgcgaggccgtgtggatcgacctcatcggcaagctggtacccgcgaccagtgtcgcccaggggcgactgctggtcgcggcggcgattagcttcatcgaagacgtcgctcgcacctggcatctcacgcgctacgccggagtcgccgacgagatcagtggcctggcgctggcgatcctgaccagcggggcaggtaacctcttgcgcgca

SEQ ID NO: 110 - DNA, 795 bp, Mycobacteria tuberculosis
atggtaatcgtggccgacaaggcggccggtcgggtcgctgatccggtcttgcggccggtgggcgcgctgggcgatttcttcgcgatgacgctcgacacgtccgtgtgcatgttcaagccgcctttcgcgtggcgtgaatacctacttcagtgctggttcgtggcgcgggtgtcgacgctgcctggggtgttgatgacgatcccatgggcggtgatctcggggtttctcttcaacgtcttgctgaccgacatcggtgccgcggacttttccggcaccggctgtgcgatcttcaccgtgaaccaaagcgccccgatcgtcacggtcttggtggtcgcgggcgcgggcgccaccgccatgtgcgccgatctgggtgcgcgcaccatccgtgaggaactcgacgcactgcgggtgatgggcatcaacccgatccaagcgctagcggctccgcgcgtgctggcggccaccacggtgtcgttggcgctgaattcggtggtgaccgcgacggggctgatcggcgcgttcttttgctcggtgtttctcatgcacgtctcggcgggggcatgggtgaccgggcttaccacgctgacccacaccgtggacgtcgtcatttcgatgatcaaggcgacgttgttcgggctgatggccggactgatcgcctgctataagggcatgtcggtcggtggcggcccggccggagtcggccgggcggtgaacgaaaccgtggtgtttgccttcatcgtcttgttcgtgatcaacatcgtcgtcaccgcggtcggcatcccattcatggtgtcc

SEQ ID NO: 111 - DNA, 813 bp, Mycobacteria tuberculosis
atgacggcagcgaaagcccttgtaagcgaatggaatcggatgggatcgcagatgcggttcttcgtcggcacgctggccgggattcccgacgccctcatgcactaccgcggcgagctgctgcgggtgatcgcgcaaatggggttggggaccggggttcttgcggtgatcggtggaacggtcgcgatcgtcgggttcttggcgatgaccaccggcgcgatcgtggccgtgcagggctacaaccagttcgcttcggtgggtgtggaggcgctgaccggcttcgcgtcggccttcttcaacacccgcgagattcagcccggaaccgtgatggtcgcgctagcggccaccgtcggtgccggtaccaccgctgcgctgggggcgatgcggataaacgaggagatcgacgcgctcgaggtgatcggcatccgcagcatcagctacctggcgagcacccgggtgctggccggagtggtcgtggccgtccctctgttctgtgtgggactgatgacggcctacctggccgcgcgcgtcggcaccaccgccatctatggccaggggtcgggcgtgtacgaccactacttcaacacgttcctgcgcccgaccgacgtgctctggtcgtcggttgaagtcgtcgtggtcgctctgatgatcatgctggtgtgcacctattacggctacgccgcacatggcgggccggccggggttggcgaggcggtcggccgggccgtgcgtgcctcgatggtcgtcgcgtcgatcgcaatccttgtcatgacgctggccatctacggccagtcgcccaactttcacctggcgacc

SEQ ID NO: 112 - DNA, 1275 bp, M. tuberculosis
atgagacgcgggccgggtcgacaccgtttgcacgacgcgtggtggacgctgatcctgttcgcggtgatcggggtggctgtcctggtgacggcggtgtccttcacgggcagcttgcggtcgactgtgccggtgacgctggcggccgaccgctccgggctggtgatggactccggcgccaaggtcatgatgcgcggtgtgcaggtcggccgggtcgcccagatcggtcggatcgagtgggcccagaacggggcgagcctcagactggagatcgaccccgaccagatccggtacatcccggccaatgtcgaggcacagatcagcgccaccaccgcattcggtgccaagttcgtcgacctggtgatgccgcaaaacccaagtcgtgcacggctgtccgctggggcggtactgcattcgaagaacgtcagcacggaaatcaacaccgtcttcgaaaacgtcgtcgacctgctcaacatgatcgacccgctgaaactgaacgccgtgctgaccgcggtcgccgacgccgttcgcgggcaaggtgaacggataggccaggccaccaccgacctcaacgaggtgctggaggcactcaacgcacgcggcgacaccatcggcggcaactggcgatcgctcaagaacttcaccgacacctatgacgcggccgcccaagacatcctgacgatcctgaacgccgccagcaccaccagtgcgaccgtcgtgaatcattcgacgcagctggatgccttgctactcaacgccatcggactatccaacgctggcaccaacctgcttggcagcagccgagacaatctcgtcggcgcggccgacatcctggcgccgaccacgagcctgctgttcaagtacaaccccgaatacacctgcttcctgcagggcgccaagtggtatctcgacaacggcggctatgcggcctggggcggggccgacgggcgcacgctacaactcgatgtggcgctactgttcggcaacgacccctatgtctatccggacaacctgccggttgtcgcggccaaggggggtcccggcggaaggccgggatgcgggccattgccggatgccacccacaacttcccggtgcgccagctggtcaccaacaccggatggggaaccgggctggacatccggcccaaccccggcatcgggcatccctgctgggccaactacttcccggtgacccgcgcggtgcccgagccgccgtcgatccgtcagtgcatccccgggccggcgatcgggcccaaccccgcggcgggggagcagcca

SEQ ID NO: 113 - DNA, 1026 bp, Mycobacteria tuberculosis
atgagggagaacctggggggcgtcgtggtgcgcctcggcgtcttcctggcggtatgcctgctgacggcgttcctgctgattgccgtcttcggggaggtgcgcttcggcgacggcaagacctactacgccgagttcgccaacgtgtccaatctgcgaacgggcaagctggtgcgcatcgccggcgtcgaggtcggcaaggtcaccaggatctccatcaaccccgacgcgacggtgcgggtgcagttcaccgccgacaactcggtcaccctcacgcggggcacccgggcggtgatccgctacgacaacctgttcggtgaccgctatttggcgctggaggaaggggccggcggactcgccgttcttcgtcccggtcacacgattccgttggcgcgcacccaaccggcgttggatctggatgccctgatcggtggattcaagccgctgtttcgtgcgctgaaccccgagcaggtcaacgcgctgagcgaacagttgctgcacgcgtttgccggacaggggcccacgatcgggtcattgctggcccagtccgcggccgtgaccaacaccctggccgaccgtgatcggctgatcgggcaggtgatcaccaacctcaacgtggtgctgggctcgctgggcgctcacaccgatcggttggaccaggcggtgacgtcgctatcagcgttgattcaccggctcgcgcaacgcaagaccgacatctccaacgccgtggcctacaccaacgccgccgccggctcggtcgccgatctgctgtcgcaggctcgcgcgccgttggcgaaggtggttcgcgagaccgatcgggtggccggcatcgcggccgccgaccacgactacctcgacaatctgctcaacacgctgccggacaaataccaggcgctggtccgccagggtatgtacggcgacttcttcgccttctacctgtgcgacgtcgtgctcaaggtcaacggcaagggcggccagccggtgtacatcaagctggccggtcaggacagcgggcggtgcgcgccgaaa

SEQ ID NO: 114 - DNA, 1230 bp, Mycobacteria tuberculosis
atgaaatccttcgccgaacgcaaccgtctggccatcggcacagtcggcatcgtcgtcgtcgccgccgttgcgctggccgcgctgcaataccagcggctgccgtttttcaaccagggcaccagggtctccgcctatttcgccgacgccggcgggctgcgcaccggcaacaccgtcgaggtctccggctatccggtgggaaaagtgtccagcatctcgctcgacggaccgggcgtgctggtggagttcaaggtcgacaccgacgtccgactcggaaaccgcaccgaagtggcaatcaaaaccaagggcttgttgggcagcaagttcctcgacgtcaccccccgcggggacggccgactcgattctccgatcccgatcgagcggaccacgtcgccctaccaactgcccgacgcccttggcgatttggccgccacgatcagcgggttgcacaccgagcggctgtccgaatcgctggccaccctggcgcagacctttgccgatacgccggcgcacttccgcaacgccatacacggggtggcccggctcgcccaaaccctcgatgagcgcgacaaccaactgcgcagcctgctggccaacgcggccaaagccaccggggtgctggccaaccgcaccgaccagatcgtcggcctggtgcgcgacacgaatgtggtcttggcgcagctgcgcacccaaagcgccgccctggaccggatctgggcgaacatctcggcggtggccgaacaactgcggggcttcatcgctgagaaccgccagcagctgcgcccggcgctggacaagctcaacggggtgctggctatcgtcgaaaaccgcaaagagcgtgtgcggcaggccatcccgctgatcaacacctatgtcatgtcgctgggtgagtcgctgtcgtcgggcccgttcttcaaggcatacgtggtgaacctgctgccgggtcagttcgtgcaaccgttcatcagcgccgcgttctccgacctggggctcgacccggccacgttgctgccgtcgcagctgaccgacccaccgaccggtcaacccggaaccccgccgttgccgatgccctacccgcgcacgggccagggcggtgagccgcggctgacgctgcccgacgcgatcaccggcaatcccggcgatccgcgctatccgtaccggccggagccgcccgcgccgccgcccggcgggccgccgcccggcccgcccgcgcagcagccgggagaccaaccg

SEQ ID NO: 115 - DNA, 1269 bp, Mycobacteria tuberculosis
gtgacaacgaaactcagacgtgcccgctcggtgttggcgaccgccctggtgctggtcgcgggcgtgatcctggccatgcgcaccgccgacgccgccgcccgcacgaccgtggtcgcctacttcgacaacagcaacggtgtgttcgccggtgacgacgtgctcattcggggcgtgccggtgggcaagatcgtcaagatcgaaccgcaaccgctgcgcgccaagatttcgttctggttcgaccgcaaataccgagtccccgccgatgccgccgcggcgatcctgtcgccgcaactggtgaccggccgggccatccagctgacaccgccgtatgccggcgggccgaccatggccgacggcacagtaatcccgcaagagcgcaccgtggtgccggtggagtgggacgacttgcgggcgcaacttcagcggctgaccgcattgctgcagcccacccggccgggcggcgtcagcacgctgggtgcgctcatcaatactgccgccgacaacctgcgcgggcaaggcgccaccatccgcgacaccatcatcaaactgtcacaagcgatttcggctctcggtgaccacagcaaagacatcttctccaccgtgacgaacctgtcgacgctggtcacggcgctgcatgacagcgctgacctgctcgaacggctcaaccacaacctggccgcggtgacctcgctgctggccgatggcccggacaagatcggtcaggcagccgaggacctcaacgcggtcgtagccgacgtcggcagcttcgccgccgagcaccgcgaggcgatcggcaccgcatcagacaagctcgcgtcaatcaccaccgcgctggtcgacagcctcgacgacatcaagcagacgctgcatatcagcccgacggtgttgcagaacttcaacaacatcttcgaaccggccaacggcgcgctgaccggcgcgctggcgggcaacaacatggccaacccaatcgccttcctgtgcggcgcgatccaggctgcctcccggctgggcggcgagcaagcggccaaattgtgcgtgcaatacctggcgccgatcgtgaagaaccgccagtacaactacccgccgctgggggcgaacctgttcgtcggggcgcaggccaggcctaacgaggtcacctacagcgaggactggctgcggcccgattacgttgcaccagttgcggacacgccgccagatccggccgcggccgtgaccgtcgatcccgcgaccggcctgcgcggcatgatgatgccgccggggggtggctcg

SEQ ID NO: 116 - DNA, 1131 bp, M. tuberculosis
gtgaggatcggcctgaccctggtgatgatcgcggccgtggtagcgagctgcggctggcgcgggctgaattcgctgccgctgcccggcacgcagggcaacggcccggggtccttcgcggtccaggcgcagctgccggatgtcaacaacatccagccgaactcgcgggtgcgggttgccgacgtgacggtcggccacgtcacgaaaatcgagcgccaaggctggcacgcgttggtgaccatgcggctggatggcgacgtcgatttgcccgccaacgcaacggccaagatcggcaccaccagcctgctgggttcctaccacatcgagctggcgccaccgaaaggcgaagcgcggcaaggcaagctgcgcgacggttcactcattgcgctgtcacacggtagcgcctacccaagcaccgagcagacgctggcagcgctgtcgctggtgctcaacggcggcggactgggccaggttcaagacatcaccgaggcgttgagcaccgcgtttgccggccgtgagcacgatctgcgcgggctgattgggcagctggacaccttcaccgcatacctcaacaaccagtccggtgacatcatcgcggccaccgacagcctcaaccgcctcgtcggcaagttcgccgaccagcaacccgtcttcgatcgggccctggccaccatccccgacgcgctcgcggtgctggccgatgagcgggacacgctcgtcgaggctgccgagcagctgagcaagttcagcgccctgaccgtcgactcggtcaacaagaccaccgcgaacctggtcaccgaactgcggcaactcggaccggtgttggagtcgctggccaattccggtccggcgctgacccgatcgctgtccctgctggccacgttcccgttcccgaacgagacgttccaaaatttccagcgcggcgaatacgccaacctgaccgcgatcgtcgacctcacgctcagccgcatcgaccagggcctgttgaccggcacccgctgggagtgtcatctgacccagctcgagctgcagtggggtcgcaccattgggcagttccccagcccgtgtaccgcgggctatcggggtaccccgggcaatccgctgacgatcgcctaccgctgggatcaggggccc

SEQ ID NO: 117 - DNA, 1311 bp, Mycobacteria tuberculosis
atgctgcatctaccgcgccgagtgatcgttcagctggccgtctttaccgtgatcgcggtgggcgtgctggccatcacgttcctgcatttcgtgaggctgccggcgatgcttttcggcgtcggccgctacacggtgacgatggagctggtcgaagccggtgggctgtatcgcaccggcaatgtcacctaccgcggctttgaggtgggccgggtggcagcggtgcggctcaccgacaccggggtgcaagcggtgctggccctgaaatcgggcatcgatatcccgtcggacctcaaggccgaggtgcacagccacaccgcgatcggcgaaacctacgtcgagttgttgccgcgcaacgccgcctcgccgccactgaagaacggcgatgtcattgcgctggccgacacctcggtgccgcccgacatcaacgacctgctcagcgcggccaacaccgcattggaggcaatacctcacgagaacctgcagaccgtcatcgacgagtcgtacaccgcggtggccgggttagggctcgaactttcccggctgatcaagggctcggcggaactggcgatcgatgctcgcgcgaatctcgatccgctggtggcgctgatcgaccgggcaggaccggtgctggattcgcagacccacacctcggatgcgatcgcggcctgggcggcacagctggccgcagtcaccggccaattgcagacacacgactcggcggtcggcgatctcatcgaccggggcggtccggcgttgggggagacgcgccaactgctcgagcggctacaacccaccgtgcccatcctgctggccaacctggtcagcgtcggccaggtcgcactcacctatcacaacgacatcgaacagctgctggtggtgttccccatggccatcgccgccgaacaggccggcatcctggccaacctcaacaccaagcaggcctaccggggccagtatctgagcttcaacctcaacctgaacctgccgccgccgtgcaccaccggctttctgccggcccagcagcggcgcattcccacgttcgaggactacccggatcgcccggccggtgatctgtactgccgggtgccccaggattcgccgtttaacgtgcgcggcgcccgcaacatcccctgtgaaaccgtgccgggcaagcgcgcacccaccgtgaagttatgcgagagcgacgcgccatacctgccgctgaacgacggctacaactggaagggcgaccccaacgccacggtgccgggtttggggtccggccaggacatcccgcagacatggcaaacgatgctgctgccgccgggcagc

SEQ ID NO: 118 - DNA, 573 bp, Mycobacteria tuberculosis
atgtcggtagcagtggattccgacgccgaggatgacgccgtatcggagatcgctgaggcagccggcgtgtcgccggccccagccaaaccatccatgtcggcgccgcggcgcatgctgctgttcggcctggtcgtcgtcgtcgctttggcggtgctgttgtgttgctggggatttcgcgtccagcgggcacgccatgcgcaggaccagcgtggtcacttcctgcaagcggcccggcagtgcgcgctgaacctaacgaccatcgactggcgcaacgccgaggcggatgtgcgccgcattctggacggcgccacaggcgagttttacaacgacttcgcccagcggtcccagcccttcgtcgaagtactgaggcacgcaaaggccagcacggtcggcacgatcaccgaggccgggctgcagacgcagaccgccgacacggcccaggcgctggtggcggtgtccgtgcaaacgtcgaatgccggcgaagccgacccggttccacgagcgtggcgaatgcgcatcaccgtgcagcgggtcggcgaccgggtcaaggtgtccgacgtcgggttcgtgccg

SEQ ID NO: 119 - DNA, 480 bp, Mycobacteria tuberculosis
gtgagctggtcgcgggtgatcgcctacgggctgctgcccgggctggcgttggcgctgacgtgtggcgcgggcttgctgaaatggcaggacggcgccgtccgcgacgccgcggttgcccgtgcggaatccgtgcgggccgcgaccgacggcaccaccgcgctgctgtcttaccggcccgacaccgtgcagcatgacctcgagagcgcgcgaagcaggctcacgggcacgttcctcgacgcctacacacagctgacccacgacgtggtgatccccggcgcacagcagaagcagatctcggccgtggccaccgtcgcggccgcggcgtcggtgtcgacttccgccgaccgcgccgtcgtcctgctgttcgtaaaccagaccatcaccgtcggcaaggacgcgccgaccaccgccgcttccagcgttcgggtgaccctcgacaacatcaacgggcgttggctgatctcgcaattcgaaccgatc

SEQ ID NO: 120 - DNA, 375 bp, Mycobacteria tuberculosis
gtgcagcgccaatcattgatgccccagcagacccttgccgccggcgttttcgtgggtgcgctgctatgcggtgtcgtgacggcggcggtgccaccacacgcacgcgccgacgtggtcgcctatctggtcaacgtgacggtacgcccgggctacaacttcgccaacgccgacgccgcgttgagttacggacatggcctctgcgagaaggtgtctcggggccgcccttacgcacagatcatcgccgacgtcaaggctgatttcgacacccgcgaccaataccaggcctcgtatctgctcagccaggctgtcaacgaactctgccccgcgctgatctggcagttgcgaaactccgcagtcgacaatcggcgctcgggc

SEQ ID NO: 121 - DNA, 663 bp, Mycobacteria tuberculosis
atgtcgcgtcgagcatcggccacgtgtgccttgtccgcgaccaccgccgtcgccataatggctgctcccgccgcacgggccgacgacaagcggctcaacgacggcgtggtcgccaacgtctacaccgttcaacgtcaggccggctgcaccaacgacgtcacgatcaacccgcaactacaattggccgcccaatggcacaccctcgatctgctgaacaaccggcacctcaacgacgacaccggttctgacggatccacaccgcaagaccgcgcgcatgccgccggcttccgcgggaaagtcgctgaaaccgtggcgatcaatcccgccgtagcgatcagcggcatcgagttgataaaccagtggtactacaaccccgcgtttttcgcgatcatgtccgactgcgccaacacccagatcggggtgtggtcagaaaacagcccggatcgcaccgtcgtggtggccgtttacggacagcccgatcgaccttccgcgatgccgcccaggggagcggtaaccggaccgccgtccccggtggccgcgcaagagaacgttcctatcgaccccagccccgactacgacgccagcgacgagatcgaatacggcatcaactggctgccatggatcctgcgcggcgtgtacccgccgcccgcaatgccgccgcag

SEQ ID NO: 122 - DNA, 405 bp, Mycobacteria tuberculosis
gtgcggtggattgtcgacggtatgaacgtgatcggaagtcgtccggatggttggtggcgcgaccgccatcgcgcgatggtgatgctggtggaaaggctcgaggggtgggccatcaccaaggctcggggcgacgacgtgacggtggtgttcgagcggccgccgtcgaccgccatcccgtcatcggtggtcgaagtggcgcatgcgcccaaggcggccgccaactcggccgacgacgagatcgtccggctggtccgatccggcgcccagccacaagagattcgtgtggtgacatcggacaaagcgttgaccgaccgggtccgagacttgggtgcggcagtctacccggcagaacggttccgtgaccttatcgacccgcgcgggtcgaacgcggcccgccgcacgcag

SEQ ID NO: 123 - DNA, 1044 bp, Mycobacteria tuberculosis
atgtctcagacacccgctacaacccgcaaaacgtttcccgagatcagctcaagagcgtgggagcaccccgccgaccggaccgccctttccgcgctgcgccggctcaaaggcttcgaccagatcttgaagctgatgtcggggatgttgcgggaacggcagcaccggctgctgtacctggccagcgcggcacgggtcgggccgcggcagttcgccgacctcgacgcgctgctggacgaatgcgtggatgtgctggacgcgtcggcgaaacccgaactctacgtgatgcagtcaccaatcgcggatgccttcaccatcggcatgggcaagccattcaccgtgatcacctcggggctgtacgacctggtgacacacgacgagatgcggttcgtgatgggccacgagctcggccacgcactgtccggccacgcggtgtaccgcacgatgatgatgcatctgctgcggttggcccggtcattcggcgtcttgccggttggcggctgggcgctgcgcgcaatcgtggctgcgctgctggaatggcagcgcaaatcggagctgtccggcgatcgcgctgggttgctgtgcgcgcaggatttggacaccgcgctcagggtggagatgaagctcgctggcggctgccggctggacaagctggactcggaggccttcttggctcaggcccgggaatacgagacatccggcgatatgcgcgacggggtgctcaagctgctcaacctggagctgcagacccatccgttctctgtgctgcgggctgccgccttgactcactgggtggacaccggcggctatgccaaggtgatagccggcgagtacccgcgtcgggccgacgacggcaacgccaaatttgcagacgaccttggcgcggccgcccggtactaccgggacggcttcgaccagtccaacgacccgctgatcaaaggtatccgcgacggattcggtggcatcgtcgagggcgtgggacgggcagcctcgaacgcggccgattcattgggccgcaagatcaccgagtggcggcagccctcgaag

SEQ ID NO: 124 - DNA, 564 bp, Mycobacteria tuberculosis
atgactacgcgtccggcaaccgaccgccgcaagatgcccactgggcgggaagaggtagcggccgcaatcctgcaggccgccaccgacctgttcgccgagcgtgggccagccgcgacgtcgattcgcgacatcgccgctcgatccaaggtcaaccacgggctggtgtttcgtcacttcggcaccaaggaccaactggttggggccgtgctcgatcacctgggcacgaagctgaccagactgttgcactccgaggcgcccgctgacatcatcgaacgggctctcgaccgacatgggcgggtcttagcccgggcactgctggacggatatcccgtgggccagctgcaacagcgatttcccaatgttgcggagctgctcgacgcggtacggcctcgctacgacagcgacttgggcgcgcggctggcggtcgcgcacgcccttgcgctgcaattcggttggcggctctttgcgcccatgctgcgctcggcgacgggtatcgacgagctgaccggtgacgaactacggctgtccgtgaacgatgcggtagcccggatcctggaaccgcac

SEQ ID NO: 125 - DNA, 702 bp, Mycobacteria tuberculosis
gtgacgatattgatcctgaccgacaacgtccacgcccatgctctggcggtcgatctgcaggccaggcatggcgatatggacgtctatcagtcccccatcggccagctgccgggtgtcccgcgatgtgatgtcgcagagcgcgtcgcggaaatcgtggagcggtatgacctcgtcctttccttccactgtaaacagaggtttcccgccgctttgatcgatggggtcaggtgtgtgaatgttcatccgggtttcaacccctacaaccgcggctggtttccccaggtcttctcgatcatcgacgggcaaaaagtcggcgtgacgatccacgagatcgacgatcagttggaccatggtccgatcatcgcccagcgggaatgcgcgatcgagtcgtgggattcctcgggaagtgtctacgcccggctgatggacatcgagcgtgagttggtgctggaacatttcgacgccatccgggacggcagctacacggctaaatcgccggccaccgagggcaacctcaacctgaaaaaggatttcgaacaactccggcggctagacctgaacgagcgcggaacgtttgggcatttcctgaatcgcctgcgcgcgttgacccatgatgatttccgcaacgcttggttcgtcgatgcgtcaggccgcaaggtgtttgtccgcgtcgtgctcgaaccggagaagcccgcggaagcc

SEQ ID NO: 126 - DNA, 1599 bp, Mycobacteria tuberculosis
atgttagccttcccttatttgatgactatgatcactccacctaccttcgacgttgcgttcatcggcagcggggccgcgtgctctatgactctgctggaaatggccgatgccctgctgagcagcccctcggcatcgcccaagttgcgcatcgcggtggtggagcgagacgagcagttctggtgcggaatcccctatggccaacgctccagcatcggatcgctggccattcagaagctcgacgatttcgccgacgagccggaaaaggccgcctaccggatctggctggagcagaacaagcagcgctggctggcgttcttccaggcagagggcggtgcggccgcggcccgctggatctgcgacaaccgcgacgcattggacggcaaccagtggggggagctctacctgccgcggtttctcttcggtgtatttctgtcggagcagatgattgccgccatcgccgcgctcggcgagcgtgacctggccgaaatcgtcaccatccgcgctgaggccatgagcgcccactccgcagacggccactaccgaatcggcctccgcccgtctggaaacggtccaacggcaattgctgcaggcaaagtggttgtggccattggcagccccccgaccaaagccatccttgcgagcgattccgaacccgcattcacctatatcaacgatttctactcccccggcggggagagcaacgttgcgcgactgcgcgattcgctcgaccgcgtcgagtcgtgggagaagcgcaacgtactggtcgtgggttccaacgccacctcgctggaagcgctctacctaatgcgtcacgacgcgcgcatccgcgcacgcgtccggtccatcaccgtcatctcgcgctccggcgtgctgccctacatgatctgcaatcagccgccggagtttgacttcccgcggctgcgcacgctgctctgtacggaagcgatcgccgcggcggatctcatgtccgcgatccgcgacgatctcgcgacggccgaagaacgctcgttgaacctggccgatttgtacgacgccgttgccgccctgtttgggcaggcgctgcacaagatggatctcgtgcagcaggaagagttcttctgcgtgcacggcatgaacttcaccaagttggtgcggcgtgcgggacgcgattgccgccaggcatccgaggagctagccgcggacggcacgctgagcctgctcgccggcgaagtactgcgcgtggatgcctgcgcgtccggccagccgttcgccaccatgacctaccgagccgcgggagccgagcatacccaccccgtccccttcgctgcggtggtgaattgtggcggtttcgaggagctggacacgtgttcctcgccgttcctggtcagcgcgatgcagaacgggctgtgccgcccgaaccgcaccaaccgtggccttctggttaacgacgacttcgaggccagcccaggtttttgcgtcatcgggcccctagtcggcggcaatttcactcccaagatccgtttttggcacgtcgagagcgcaccgcgcgtccggtcgctggcgaaatcgctggcggccagcctgcttgcttcgctccagcccgtcgcactggccccatgc

SEQ ID NO: 127 - DNA, 1236 bp, Mycobacteria tuberculosis
atgaagatccgaacgttatccggctcggtgctggagccgccgtccgcagtacgcgcgaccccaggcacgtccatgttaaaactcgagccgggtggctcgacgatccccaagatccccttcatccgcccgagctttcccgggccagccgagctcgccgaggacttcgtacagatcgcccaggctaactggtacacgaacttcggtccgaacgagcggcggtttgcccgcgccctgcgcgactatctgggacctcatctgcacgttgctaccctcgccaacggcaccctggcactcctcgcggcgctccacgtcagtttcggcgccggtacgcgggaccgctacctgctgatgccgtcgttcacgttcgtcggcgtggctcaggctgcgctatggactgggtaccgtccctggttcatcgacatcgacgccaacacatggcagccatgcgtccactccgcccgcgccgtcatcgaacgcttccgcgaccggatcgccggcatcctgctggccaatgtgttcggcgtcggcaatccccagatcagcgtctgggaggagctcgccgccgaatgggagctaccgattgtgctcgactcggcggccggcttcggctccacgtacgccgacggcgagcgcctcggtggacgcggtgcatgcgagatcttctccttccatgcgaccaagccgttcgcggttggtgagggcggcgctctggtttctcgcgatccacggctcgtcgagcacgcatacaagttccagaacttcggcttggtgcaaacacgcgagtccatccagctcggaatgaacggcaagctgtcggagatcagcgccgctattggcctacgccaactagtcgggcttgatcgccgcctggcaagtcgccgcaaggtcctcgagtgctatcgcaccggtatggccgacgcgggtgtgcgtttccaggacaacgccaatgttgcgtcgctctgtttcgcgagcgcttgctgcacgtccgccgaccacaaggccgcggttctgggtagcctgcgtaggcacgcgatcgaggcgcgcgactactacaacccaccgcagcaccgacatccgtactttgtgacgaatgccgagttagtcgagtcgaccgatctagccgtcacggcggacatttgctcgcgaatcgtgtcgctgccagtccacgaccacatggccccggatgacgttgcccgggtcgtcgccgccgtgcaggaagcggaggtgcgcggtgaa

SEQ ID NO: 128 - DNA, 2358 bp, Mycobacteria tuberculosis
atgatcaccgaggacgccttccccgtcgaaccgtggcaggtccgcgagaccaagctcaacctgaacctgctggcccagtccgaatccctattcgccttgtccaacgggcacattggattacgcggcaacctcgacgagggcgaacccttcggactgccgggcacctacctgaactctttctacgaaatccggccgctgccgtacgccgaggccggttatggatatccggaggccggccagaccgttgtcgacgtcaccaacggcaagatctttcgcctgttggtcggcgacgagccgttcgacgtccggtatggcgaattgatctcccacgaacggatcctcgacctgcgcgccgggacgctgacccgccgcgcgcactggcgctcaccggcgggcaagcaagtcaaagtgacgtccacccggctggtgtcgctggcccaccgcagcgtcgcggcgatcgagtacgtcgtcgaggcaatcgaggaattcgttcgcgtgaccgtgcagtccgaactcgtcaccaacgaggacgtaccggagacctcggccgacccgcgggtgtcggccatcctggacaggccgctacaggccgtcgagcacgaacgcaccgagcggggtgcacttctcatgcaccgcacccgagccagcgcgctgatgatggccgcagggatggaacacgaggtcgaggttcccgggcgggtcgagatcaccaccgacgcccgcccggacctggcccgaaccaccgtgatctgcgggctgcgcccgggacagaagctgcgcatcgtcaaatacctggcctatggctggtccagcctgcgctcccgcccggcgctgcgcgaccaggccgccggcgcgctgcacggtgcccgctacagcggctggcaggggctgctggacgcgcaacgcgcctacctcgacgacttctgggacagcgcggacgtggaggtcgagggcgacccggaatgtcagcaagcggtgcgtttcgggttatttcacctgttgcaggccagcgcgcgcgccgaacgccgcgcgatccccagcaaggggctcaccggaaccgggtatgacggccacgccttttgggacaccgaaggtttcgtgctaccggtgctcacctacaccgcaccgcatgcggtcgccgacgcgctgcggtggcgggcgtcgacgttggacctggccaaggagcgggcggccgagctcggcctggaaggtgccgcctttccctggcggaccatccgcggacaggagtcctcggcctactggccggccggcacggcggcctggcacatcaacgccgacatcgcgatggcgttcgagcggtaccgcatcgtcaccggcgacggttcgctggaggaggaatgcggccttgcggtgctgatcgagaccgcccggctgtggctctcgctcgggcaccacgaccgccacggcgtctggcacctcgacggggtcaccggtcccgacgagtacacggcggtcgtccgcgacaacgtgttcacgaatctgatggcggcgcacaatctgcacaccgccgccgatgcttgcttgcgccaccccgaggcggcggaggccatgggtgtcaccaccgaggagatggccgcctggcgcgacgcggccgacgccgccaacattccctacgacgaggaactcggtgtccaccagcagtgtgaagggttcaccacccttgcggagtgggatttcgaagccaacaccacttatccgttgctactgcacgaggcctacgtgcgcttgtatcccgcacaggtgatcaagcaggccgacctggtgctggcgatgcagtggcagagtcacgcgttcacgcccgagcagaaggcgcgcaacgtcgactactacgaacggcgcatggtgcgcgactcgtcgttgtcggcctgcactcaggcggtgatgtgcgccgaggtcggccatctcgagttggcccacgactatgcctacgaagccgccctgatcgacctgcgcgacctgcaccgcaacacccgtgacggcctacacatggcttcgctggccggagcctggacggcgctggtcgtaggcttcggcggcctacgcgacgacgagggcatcctgtccatcgatccgcagctgcccgacggcatctcgcggctgcggttccggctgcgatggcgcggcttccggctgatcgtcgacgccaaccacaccgacgtcaccttcatccttggcgacggtcccggcacccagctgaccatgcgccacgccggccaagatctgacgctgcacacggacacaccgtccaccatcgccgtgcgcacccgtaagccgctgctgccgccaccaccgcagccgccaggccgcgagccagtgcaccgccgggctttagcccgg

SEQ ID NO: 129 - DNA, 786 bp, Mycobacteria tuberculosis
atggcgaactggtatcgcccgaactatccggaagtgaggtcccgcgtgctgggtctgcccgagaaggtgcgtgcttgcctgttcgacctcgacggtgtgctcaccgataccgcgagcctgcataccaaggcgtggaaggccatgtttgacgcctacctagccgagcgagccgagcgcaccggcgaaaaattcgttcccttcgaccctgccgcggactatcacacgtatgtggacggcaagaaacgcgaagacggcgttcgatcgtttctgagcagccgcgccatcgaaatacccgacggttccccggatgacccgggcgccgccgagacggtgtatggcctgggcaaccgcaagaacgacatgttgcacaagctgctgcgcgacgatggggcccaggtgttcgacgggtcgcggcgctacctggaggcggtcacggccgcgggtctcggtgtggccgtggtgtcttcgagcgccaacacccgcgacgtgctcgcgaccaccggtctggaccggttcgtccagcagcgggtggacggcgtgacgttgcgcgaagagcacatcgccggcaagccggcccccgactccttcctgcgcgcggcagaactgttgggggttacccccgacgcggcggcggtgttcgaggacgccctgtccggggtggcggccggccgcgccggcaacttcgccgtagtggtgggcatcaaccgaacgggccgggcggctcaggccgcccagttgcgccgccatggcgccgacgtggtggtaaccgatctcgccgagctgctg

SEQ ID NO: 130 - DNA, 60 bp, M. tuberculosis
  misc_feature (1)...(60): n = A,T,C or G
antagtaatgtgcgagctgagcgatgtcgccgctcccaaaaattaccaatggttnggtca

SEQ ID NO: 131 - DNA, 60 bp, M. tuberculosis
agtagtaatgtgcgagctgagcgatgtcgccgctcccaaaaattaccaatggtttggtca

SEQ ID NO: 132 - DNA, 60 bp, M. tuberbulosis
tgacgccttcctaaccagaattgtgaattcatacaagccgtagtcgtgcagaagcgcaac

SEQ ID NO: 133 - DNA, 60 bp, M. tuberculosis; identical to SEQ ID NO: 132

SEQ ID NO: 134 - DNA, 11 bp, M. tuberculosis
actcttggagt

SEQ ID NO: 135 - DNA, 11 bp, M. tuberculosis
actcttggagt

SEQ ID NO: 136 - DNA, 49 bp, M. tuberculosis
  misc_feature (1)...(49): n = A,T,C or G
gtggcctacaacggngctctccgnggcgcgggcgtaccggatatcttag

SEQ ID NO: 137 - DNA, 49 bp, M. tuberculosis
gcggcctacaacggcgctctccgcggcgcgggcgtaccggatatcttag